(19) United States
(12) Patent Application Publication
(10) Pub. No.: US 2005/0071891 A1
(43) Pub. Date: Mar. 31, 2005

(54) Repressible sterility of animals

(76) Inventors: Ron Thresher, Tasmania (AU); Lyn Hinds, Australian Capital Territory (AU); Chris Hardy, Australian Capital Territory (AU); Steve Whyard, Australian Capital Territory (AU); Soma Vignarajan, Australian Capital Territory (AU); Peter Martin Grewe, Tasmania (AU); Jawahar Patil, Tasmania (AU)

Correspondence Address: WILLIAMS, MORGAN & AMERSON, P.C., 10333 RICHMOND, SUITE 1100, HOUSTON, TX, 77042, US

(21) Appl. No.: 10/169,050

(22) PCT Filed: Dec. 22, 2000
(86) PCT No.: PCT/AU00/01596

(30) Foreign Application Priority Data
Dec. 24, 1999 (AU) PQ4884

(51) Int. Cl. A01K 67/00; C12N 15/85

(52) U.S. Cl. 800/8; 435/455

(57) ABSTRACT

A construct which allows animals to be bred in captivity but renders them infertile in the wild by allowing reversible control over fertility and reproduction. The construct comprises: a first promoter that is activated in a defined spatial (tissue specific) or temporal manner linked to DNA encoding a transactivating protein; and a second promoter, which is activated by the transacting protein, linked to DNA encoding a blocker molecule which disrupts gametogenesis or embryogenesis. Feeding an animal a molecule that prevents the transactivating protein binding the second promoter controls fertility.

### FIELD OF THE INVENTION

This application is concerned with the control of animal reproduction, and especially with preventing the spread of feral and/or genetically modified animals. In particular, the present invention relates to constructs and methods that allow animals to be bred in captivity, but renders them infertile in the wild, by allowing reversible control over fertility and reproduction.

### BACKGROUND OF THE INVENTION

Feral animals are one of the world's major environmental problems. Goats, cats, rabbits and carp are only the more prominent of hundreds of species traded internationally for recreation or agriculture that have escaped into the wild and formed destructive populations. Terrestrial, freshwater and marine ecosystems are all conspicuously degraded by these species, to the extent that public concern over feral animals has become a major issue for industries seeking to introduce new species in order to compete on world markets.

A good recent example is the Pacific oyster. Despite the promise of new jobs in coastal communities and an industry that is worth $50-75 million annually, recent applications to expand the geographic area for Pacific oyster mariculture facilities in Australia and the United States have been rejected indefinitely until the problem of feral oysters can be overcome. Even plans to expand the size of the industry in areas where farming already occurs are being blocked for the same reason, following very public and often acrimonious debate between industry and conservation-minded elements of the community. Attempts to solve the problem using current techniques such as triploidy and sterile hybrids have not been successful. Neither technique can guarantee a zero risk of producing feral populations, and both also suffer major technical difficulties. In the case of oysters, for example, animals sterilised via chemical or genetic manipulation of ploidy do not produce significant amounts of roe, which substantially reduces their market value. Moreover, these animals still produce a small number of viable gametes. So the debate continues to focus on whether degraded beaches are an acceptable price for new industries and jobs.

Hundreds of species of exotic animals are shipped internationally each day, mainly for recreational purposes. Inevitably, either accidentally and/or through intentional release, some animals will escape, and establish feral populations. Sterilisation prior to importation of such exotics would prevent the establishment of feral populations and remove the risk of forming new problem pest species. A generic means of sterilisation that prevents development of these feral populations would have huge economic and environmental benefits.

More recently, the containment of genetically modified animals has caused concern. For example, Salmon containing genes for enhanced production of growth hormones have been produced in Europe, New Zealand and North America. Concern has been expressed about the impact of these fish as “super-competitors”, should they escape and form feral populations. Similar concerns have been expressed about other genetic improvements that deliberately or accidentally enhance competitiveness. This concern has now grown to a point where there is pressure to ban such modified organisms in toto. However, given their economic significance, it may be preferable to have effective biological controls in place which enable these organisms to be contained within a specific locality. A sterile feral construct inserted into the genetically enhanced stock would prevent development of viable feral populations, as well as preventing integration of enhanced genes into populations of wild con-specifics.

Accordingly, some of the major benefits that a sterile feral construct would offer include:
1. Provision of a fail-safe system for preventing the establishment of feral populations of exotic species. This could fundamentally change the risk of importing these species, and would reduce public antagonism to farming of those that have the potential to be environmentally destructive.
2. Protection of investments in breeding stocks, for example those developed by extensive selective breeding programs. Currently, the commercial advantages from improved stock can be lost when live, reproductively capable animals are marketed (eg oysters, prawns, and sheep). Repressible sterility can be used as a “lock and key” process whereby improved stock could only breed when provided the correct combination of repressers (and optionally inducers) in exactly the right sequence.
3. Production of animals for intentional release that are guaranteed to be sterile. Release of such sterile animals has been used as a control mechanism for certain highly fecund pest species, eg. insects. Repressible sterility technology makes it possible to apply similar approaches to other, existing pest species, for which there are currently no “sterile male” equivalents.
4. Provision of an effective containment mechanism for genetically modified organisms. Repressible sterility provides just such a security system for future applications of molecular engineering in animal production, yet enables safe propagation of these individuals using conventional rearing facilities. Linking a genetically engineered process (faster growth, longer spawning seasons, etc.) to a repressible sterility construct ensures that genetic enhancements of exotic or native species do not enter wild populations.

One method of containing genetically-modified organisms, namely, plants, is the so-called “terminator gene” or Technology Protection System (TPS). This approach was developed by Delta and Pine Land Company (D&PL), who jointly owns the rights for this invention with USDA-ARS, as disclosed in U.S. Pat. No. 5,723,765, which is incorporated herein by reference. Essentially, the method stops the seeds of certain plants from germinating, and utilizes:
1. A transiently-active promoter operably linked to a first (toxic, hence lethal) gene, but separated by a blocking sequence which prevents the lethal gene expression;
2. A second gene, encoding a recombinase which, upon expression, excises the blocker sequence; and
3. A third gene, encoding a tetracycline-controllable blocker of the recombinase.

Unless the seeds of the plants are transformed with all three genes, and receive the tetracycline at a precise point, the recombinase is expressed, resulting in the blocker sequence being excised, and the toxic gene being expressed.

While this method may function well in plants, it would not function in many animal species. Few recombinases have been identified that will function in animals (and vertebrates in particular) and those that have been identified (eg., Cre and Flp recombinase) function in only a limited number of species. Moreover, the use of a toxic substance in animals may be unacceptable, particularly for those likely to be consumed. Further, the system requires a number of complex steps, which are not readily achieved, and once the blocker sequence has been excised it is virtually impossible to reverse the control process.

Accordingly, there is still a need to provide methods of preventing the escape of exotic and/or genetically modified animals.

We have now developed such a method. We have designed certain genetic constructs that allow animals to be bred in captivity, but render them reproductively non-viable or infertile in the wild. Moreover, these constructs provide reversible control over fertility and reproduction, and are applicable to a wide variety of animal species.

### SUMMARY OF THE INVENTION

In its most general aspect, the invention disclosed herein provides a nucleic acid construct which may be inserted into the genome of any target organism. The construct can use any promoter/gene combinations, provided that they satisfy the criteria of being activated only during embryonic development and/or gametogenesis, and being crucial for completion of embryogenic development and/or gametogenesis.

One type of construct, which is designed to function in a variety of target species, comprises:
a) a native promoter of a crucial gene;
b) a blocking DNA sequence (blocker) contoured for and designed to abrogate the crucial gene's function or to cause its mis-expression; and
c) a genetic switch to regulate controlled expression/repression of the blocker/gene knockout.

In captivity, expression of the blocker can be repressed in the presence of a trigger molecule, supplied via the diet or in soluble form, so that fertilisation occurs and embryos complete development. In the wild, where the trigger molecule is unavailable, the blocker remains active and the critical gene is disrupted, leading to early death of invasive progeny.

Accordingly, in a first aspect, the present invention provides a construct for disrupting gametogenesis or embryogenesis in animals, comprising:
a) a first nucleic acid molecule, which is activated in a defined spatio-temporal pattern, and which is operably linked to
b) a second nucleic acid molecule, which encodes a transactivating protein; and
c) a third nucleic acid molecule, which is operably linked to a fourth nucleic acid molecule, wherein activation of said first nucleic acid molecule controls the expression of the second nucleic acid molecule, which in turn activates the third nucleic acid molecule, which effects the expression of the fourth nucleic acid molecule which encodes a blocker molecule which disrupts gametogenesis or embryogenesis in the animal. Either or both the first and fourth nucleic acid molecules are transiently activated or transiently affect development in a defined spatio-temporal pattern.

Each of the first, second, third and fourth nucleic acids may be genomic DNA, cDNA, RNA, or a hybrid molecule thereof. It will be clearly understood that the term nucleic acid molecule encompasses a full-length molecule, or a biologically active fragment thereof.

Preferably the first nucleic acid molecule is a DNA molecule encoding a promoter region. More preferably the promoter is activated only during embryonic development and/or gametogenesis, and is crucial for completion of embryogenic development and/or gametogenesis. Most preferably this DNA molecule has the nucleotide sequence shown in SEQ ID NO:1, SEQ. ID NO:8 SEQ ID NO:60. A sample of SEQ ID NO.1 DNA was deposited at the Australian Government Analytical Laboratories on 22 Dec. 1999, and accorded the accession number MM99/09098. A sample of SEQ ID NO.8 DNA was deposited at the Australian Government Analytical Laboratories on ______, and accorded the accession number ______. A sample of SEQ ID NO.60 DNA was deposited at the Australian Government Analytical Laboratories on 23 Dec. 1999, and accorded the accession number NM99/09106.

Preferably the second nucleic acid molecule is a cDNA molecule encoding the tetracycline-responsive transcriptional activator protein (tTA), as defined herein, having a nucleotide sequence of SEQ ID NO:2. A sample of SEQ ID NO.2 cDNA was deposited at the Australian Government Analytical Laboratories on 22 Dec. 1999, and accorded the accession number MM99/09099.

Preferably the third nucleic acid molecule is DNA molecule encoding a repressible promoter. More preferably the promoter consists of the tet responsive element (TRE) which is coupled to and tightly regulates a minimal promoter region. Most preferably this comprises the tet responsive element (TRE) and the P_{minCMV} as shown in SEQ ID NO:3. A sample of SEQ ID NO.3 DNA was deposited at the Australian Government Analytical Laboratories on 22 Dec. 1999, and accorded the accession number MM99/09100.

Preferably the fourth nucleic acid molecule encodes a blocker molecule selected from the group consisting of antisense RNA, double-stranded RNA (dsRNA), sense RNA and ribozyme. More preferably the molecule is dsRNA or sense RNA that when mis-expressed disrupts development in a defined spatio-temporal pattern. Most preferably this RNA molecule is encoded by the nucleotide sequence shown in SEQ ID NO:13, SEQ ID NO:62, SEQ ID NO:23, SEQ ID NO:24, and SEQ ID:61. A sample of SEQ ID NO.13 DNA was deposited at the Australian Government Analytical Laboratories on 22 Dec. 1999, and accorded the accession number MM99/09100. A sample of SEQ ID NO:62 DNA was deposited at the Australian Government Analytical Laboratories on ______, and accorded the accession number ______. A sample of SEQ ID NO.23 DNA was deposited at the Australian Government Analytical Laboratories on 22 Dec. 1999, and accorded the accession number NM99/09101. A sample of SEQ ID NO.24 DNA was deposited at the Australian Government Analytical Laboratories on 22 Dec. 1999, and accorded the accession number NM99/09102. A sample of SEQ ID NO.61 DNA was deposited at the Australian Government Analytical Laboratories on 23 Dec. 1999, and accorded the accession number NM99/09107.

In a second aspect, the present invention provides a nucleic acid molecule, which encodes a promoter and is transiently activated in a defined spatio-temporal pattern. More preferably, the promoter is active only during a narrow window during embryogenesis or larval development. Most preferably the nucleic acid is a promoter having a nucleotide sequence as shown in SEQ ID NO:1, SEQ ID NO:8 and SEQ ID NO:60.

In a third aspect, the present invention provides a nucleic acid molecule, which encodes a promoter having:
a) a nucleotide sequence as shown in SEQ ID NO:1, SEQ ID NO:8 and SEQ ID NO:60; or
b) a biologically active fragment of the sequence in a); or
c) a nucleic acid molecule which has at least 75% sequence homology to the sequence in a) or b); or
d) a nucleic acid molecule which is capable of hybridizing to the sequence in a) or b) under stringent conditions.

In a fourth aspect, the present invention provides a nucleic acid molecule that encodes the coding region of a gene including:
a) a nucleotide sequence selected from the group consisting of SEQ ID NO:63, SEQ ID NO:23, SEQ ID NO:24 and SEQ ID NO 61 or
b) a biologically active fragment of any one of the sequences in a); or
c) a nucleic acid molecule which has at least 75% sequence homology with any one of the sequences disclosed in a) or b); or
d) a nucleic acid molecule that is capable of binding to any one of the sequences disclosed in a) or b) under stringent conditions.

A sample of SEQ ID NO.63 DNA was deposited at the Australian Government Analytical Laboratories on 22 Dec. 1999, and accorded the accession number MM99/09100. A sample of SEQ ID NO.23 DNA was deposited at the Australian Government Analytical Laboratories on 22 Dec. 1999, and accorded the accession number NM99/09101. A sample of SEQ ID NO.24 DNA was deposited at the Australian Government Analytical Laboratories on 22 Dec. 1999, and accorded the accession number NM99/09102. A sample of SEQ ID NO.61 DNA was deposited at the Australian Government Analytical Laboratories on 23 Dec. 1999, and accorded the accession number NM99/09107.

In a fifth aspect, the present invention provides a nucleic acid molecule which encodes a blocker molecule wherein the blocker molecule is capable of disrupting gametogenesis or embryogenesis in an animal.

Preferably the blocker molecule is selected from the group consisting of antisense RNA, dsRNA, sense RNA and ribozyme. More preferably the molecule is dsRNA or sense RNA that when mis-expressed disrupts development in a defined spatio-temporal pattern. Most preferably the blocker molecule is encoded, or partially encoded, by a sequence selected from the group consisting of SEQ ID NO:13, SEQ ID NO:62, SEQ ID NO:23 and SEQ ID NO:61. A sample of SEQ ID NO.13 DNA was deposited at the Australian Government Analytical Laboratories on 22 Dec. 1999, and accorded the accession number MM99/09100. A sample of SEQ. ID NO.62 DNA was deposited at the Australian Government Analytical Laboratories on ______, and accorded the accession number ______. A sample of SEQ ID NO.61 DNA was deposited at the Australian Government Analytical Laboratories on 23 Dec. 1999, and accorded the accession number NM99/09107.

In an sixth aspect, the present invention provides a construct for disrupting gametogenesis or embryogenesis in animals, comprising:
a) a first nucleic acid molecule, which is transiently activated in a defined spatio-temporal pattern, and which is operably linked to
b) a second nucleic acid molecule, which encodes a blocker molecule.
   wherein activation of said first nucleic acid molecule controls the expression of the second nucleic acid which disrupts gametogenesis or embryogenesis in the animal.

In a seventh aspect, the present invention provides a method of preventing embryogenesis in animals comprising the steps of:
1) stably transforming an animal cell with a construct according to the invention; and
2) implanting the cell into a host organism, whereby a whole animal develops from the implanted cell.

Preferably, the stable transformation is effected by microinjection, transfection or infection, wherein the construct stably integrates into the genome by homologous recombination.

In an eighth aspect, the present invention provides a transgenic animal stably transformed with a construct according to the invention.

Preferably the host organism is of the same genus as the transformed cell. More preferably the host organism is any animal, including vertebrates and invertebrates. Most preferably the host organism is selected from the group consisting of fish, mammals, amphibians, and mollusc. Fish include; but are not limited to, zebrafish, European carp, salmon, tilapia and trout. Mammals include; but are, not limited to, cats, dogs, donkeys, camels, rabbits, rats, and mice. Molluscs include; but are not limited to, Pacific oysters, zebra mussels, striped mussels, abalone, pearl oysters, and scallops.

Modified and variant forms of the constructs may be produced in vitro, by means of chemical or enzymatic treatment, or in vivo by means of recombinant DNA technology. Such constructs may differ from those disclosed, for example, by virtue of one or more nucleotide substitutions, deletions or insertions, but substantially retain a biological activity of the construct or nucleic acid molecule of this invention.

### BRIEF DESCRIPTION OF THE FIGURES

FIG. 1 shows the plasmid map of pBAC5/H11.

FIG. 2 shows the plasmid map of pZBMP2(1.4)-EGFP. The transcriptional unit consists of the modified EGFP coding sequences (Cormac et al., 1996), under the regulation of a 1,414 bp zBMP2 promoter.

FIG. 3 shows zBMP2 promoter-driven EGFP expression in zebrafish embryo at 9.5 h pi. Right, latero-ventral view, anterior to right. Panel A shows a typical zebrafish embryo showing EGFP expression predominantly in the anterio-ventral region. Panel B shows a light micrograph of the embryo on left. PO, polster.

FIG. 4 shows EGFP expression in 9.5 hpi old zebrafish embryo. Lateral views, with dorsal to top and anterior to left. Panel A shows EGFP expression driven by zBMP2 promoter. Panel B shows a light micrograph of the embryo on left. PO, polster; TB, tail bud.

FIG. 5 shows anterior region of a zebrafish embryo, showing EGFP expression driven by zpBMP2 at 24-h pi. Panel A shows the left, dorso-lateral view. EGFP expression is seen in domains of native zBMP2 expression. Panel B shows light micrograph of the embryo on left. Left, lateral view. PE, posterior margin of eye; OV, otic vesicle; FB, pectoral fin bud.

FIG. 6 shows the plasmid map of pSMAD5-EGFP. A sample of pSMAD5-EGFP was deposited at the Australian Government Analytical Laboratories on ______, and accorded the accession number ______. The zebrafish smad5 promoter drives expression of the EGFP.

FIG. 7 shows a shield stage zebrafish embryo, showing ubiquitous expression of EGFP (panel A) driven by zebrafish smad5 promoter Panel B represents the light micrograph of the embryo on left.

FIG. 8 shows middle section of a typical 24 hpi zebrafish embryo injected with pSMAd5-EGFP. The EGFP expression is predominantly restricted to ventral tissues. D, dorsal; V, ventral.

FIG. 9 shows dorsalized phenotypes of zebrafish, resulting from zBMP2 antisense (A) and dsRNA (B) injections. Developments of ventral structures are perturbed in both instances.

FIG. 10 shows the ventralized chordino phenotypes of zebrafish resulting from zBMP2 sense transcript injections. Enlarged blood island (A and B, arrow) and multiplicated ventral margin of tail fin (C, arrow).

FIG. 11 shows the plasmid map of the antisense EGFP fusion construct, pzBMP2-As-EGFP. A sample of pzBMP2-As-EGFP was deposited at the Australian Government Analytical Laboratories on 22 Dec. 1999, and accorded the accession number MM99/09102.

FIG. 12 shows the plasmid map of pzBMP2-dsRNA. The zBMP2 promoter drives the expression of about 800 bp of zBMP2 cDNA, designed to fold back on itself as a dsRNA.

FIG. 13 shows the plasmid map of pzBMP2-Tet-Off. This construct was engineered to drive expression of tTA under the regulation of zBMP2 promoter.

FIG. 14 shows the plasmid map of the complete sterile feral construct, pSF1. The zBMP2 promoter drives the expression of tTA, which in turn activates the expression of EGFP and the zBMP2 double stranded RNA blocker, in the absence of doxycycline.

FIG. 15 shows a plasmid map of zebrafish Sterile feral Construct pSF2. This construct is identical to pSF1, except that CMV promoter drive's the tTA. A sample of pSF2 was deposited at the Australian Government Analytical Laboratories on ______, and accorded the accession number ______.

FIG. 16 shows a plasmid map of zebrafish Sterile feral Construct pSF3. This construct is identical to pSF2, except that the zebrafish smad5 promoter drives the tTA. A sample of pSF3 was deposited at the Australian Government Analytical Laboratories on ______, and accorded the accession number ______.

FIG. 17 shows a plasmid map of zebrafish Sterile feral Construct pSF4. This construct is identical to pSF3, except that the zBMP2 double stranded RNA blocker is replaced by zBMP2 sense cDNA. A sample of pSF4 was deposited at the Australian Government Analytical Laboratories on ______, and accorded the accession number

FIG. 18 (A-C) show 24-hpi zebrafish embryos following the injection of pSF4. Panel A, two-zebrafish embryos with enlarged blood islands (arrow), typical of ventralized mutations. Panel B, close up view of 24 hpi zebrafish embryo tail, with enlarged blood island (arrow). Panel C, EGFP micrograph of embryo in panel B, showing close association of EGFP expression and ventralization (arrow).

FIG. 19 shows the amino acid alignments of closely related HOXCG1 and HOXCG3 genes in various animals.

FIG. 20 shows (a) typical control D-hinge larvae with a single velum and (b) a larvae exhibiting the multiple velum phenotype as a consequence of blocking expression of Hox CG1 with double stranded HOXG1 RNA.

FIG. 21 shows the plasmid map of the double stranded blocking construct for oyster Hox gene, pBiT(dHSP)-RFP-oHoxDS/BH. A sample of pBiT(dHSP)-RFP-oHoxDS/BH was deposited at the Australian Government Analytical Laboratories on ______, and accorded the accession number ______.

FIG. 22 shows the amino acid alignments of closely related goosecoid genes in various animals.

FIG. 23 shows the mechanisms of action of regulatory elements of the mouse goosecoid gene promoter region.

FIG. 24 shows the plasmid map of the mouse goosecoid promoter driving expression of the enhanced green fluorescent protein reporter (pSFM 1).

FIG. 25 shows the plasmid map of the tetracycline transactivated TRE driving expression of the mouse goosecoid cDNA (pSFM 2).

FIG. 26 shows the mouse goosecoid promoter driving expression of mouse goosecoid cDNA fused to the red fluorescent protein reporter (pSFM 6).

FIG. 27 shows the plasmid map of the mouse goosecoid promoter driving expression of the tetracycline transactivator tTA protein (pSFM 7).

FIG. 28 shows the plasmid map of the mouse goosecoid promoter driving expression of the luciferase+ protein reporter (pSFM 20).

FIG. 29 shows the plasmid map of the promoter-less luciferase+ protein reporter (pSFM 21).

FIG. 30 shows the plasmid map of the CMV promoter driving expression of the luciferase+ protein reporter (pSFM 23).

FIG. 31 shows the plasmid map of the tetracycline transactivated TRE driving expression of the enhanced green fluorescent protein reporter (pSFM 24).

FIG. 32 shows the plasmid map of the tetracycline transactivated TRE driving expression of the luciferase+ protein reporter (pSFM 25).

FIG. 33 shows an agarose gel demonstrating the presence of mouse goosecoid mRNA expression in P19 cells as detected by RT-PCR amplification of mRNA using goosecoid-specific primers. Lane 1: PCR product from P19 cells using goosecoid primers; Lane 2: PCR product from 1 fg of pSFM 2 as a positive goosecoid control; Lane 3: PCR product from P19 cells with GAPDH primers; Lane 4: DNA MW marker.

FIG. 34 shows the plasmid map of the tetracycline transactivated TRE driving expression of the mouse goosecoid dsRNA blocker construct (pSFM 5).

FIG. 35 shows the plasmid map of the CMV promoter driving expression of the mouse goosecoid antisense RNA blocker construct (pSFM 8).

FIG. 36 shows the plasmid map of the tetracycline transactivated TRE driving expression of the mouse goosecoid antisense blocker construct (pSFM 9). A sample of pSFM 9 was deposited at the Australian Government Analytical Laboratories on 23 Dec. 1999 and accorded the accession number NM99/09107.

FIG. 37 shows the cellular locations of CMV promoter-driven expression of red fluorescent protein in P19-SFM 7 cells (A,B), CMV promoter-driven expression of red fluorescent protein fused to the mouse goosecoid protein (C) and TRE tetracycline responsive enhanced green fluorescent protein expression in cells co-transfected with CMV promoter-driven expression of red fluorescent protein fused to the mouse goosecoid protein (D).

### DETAILED DESCRIPTION OF THE INVENTION

The practice of the present invention employs, unless otherwise indicated, conventional molecular biology, microbiology, and recombinant DNA techniques within the skill of the art. Such techniques are well known to the skilled worker, and are explained fully in the literature. See, e.g., “DNA Cloning: A Practical Approach,” Volumes I and II (D. N. Glover, ed., 1985); “Oligonucleotide Synthesis” (M. J. Gait, ed., 1984); “Nucleic Acid Hybridization” (B. D. Hames & S. J. Higgins, eds., 1985); “Transcription and Translation” (B. D. Hames & S. J. Higgins, eds., 1984); “Animal Cell Culture” (R. I. Freshney, ed., 1986); “Immobilized Cells and Enzymes” (IRL Press, 1986); B. Perbal, “A Practical Guide to Molecular Cloning” (1984), and Sambrook, et al., “Molecular Cloning: a Laboratory Manual” 12^{th} edition (1989).

Definitions

The description that follows makes use of a number of terms used in recombinant DNA technology. In order to provide a clear and consistent understanding of the specification and claims, including the scope given such terms, the following definitions are provided.

A “nucleic acid molecule” or “polynucleic acid molecule” refers herein to deoxyribonucleic acid and ribonucleic acid in all their forms, i.e., single and double-stranded DNA, cDNA, mRNA, and the like.

A “double-stranded DNA molecule” refers to the polymeric form of deoxyribonucleotides (adenine, guanine, thymine, or cytosine) in its normal, double-stranded helix. This term refers only to the primary and secondary structure of the molecule, and does not limit it to any particular tertiary forms. Thus this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA).

A DNA sequence “corresponds” to an amino acid sequence if translation of the DNA sequence in accordance with the genetic code yields the amino acid sequence (i.e., the DNA sequence “encodes” the amino acid sequence).

One DNA sequence “corresponds” to another DNA sequence if the two sequences encode the same amino acid sequence.

Two DNA sequences are “substantially similar” when at least about 85%, preferably at least about 90%, and most preferably at least about 95%, of the nucleotides match over the defined length of the DNA sequences. Sequences that are substantially similar can be identified in a Southern hybridization experiment, for example under stringent conditions as defined for that particular system. Defining appropriate hybridization conditions is within the skill of the art. See e.g., Sambrook et al., “Molecular Cloning: a Laboratory Manual” 12^{th} edition (1989), vols. I, II and III. Nucleic Acid Hybridization. However, ordinarily, “stringent conditions” for hybridization or annealing of nucleic acid molecules are those that (1) employ low ionic strength and high temperature for washing, for example, 0.015 M NaCl/0.0015 M sodium citrate/0.1% sodium dodecyl sulfate (SDS) at 50° C., or (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C.

Another example is use of 50% formamide, 5×SSC (0.75 M NaCl, 0.075 M sodium citrate), 50 mM sodium phosphate (pH 6.8), 0.1% sodium pyrophosphate, 5× “Denhardt's solution, sonicated salmon sperm DNA (50 μg/mL), 0.1% SDS, and 10% dextran sulfate at 42° C., with washes at 42° C. in 0.2×SSC and 0.1% SDS.

A “heterologous” region or domain of a DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous region is a construct where the coding sequence itself is not found in nature (e.g., a cDNA where the genomic coding sequence contains introns, or synthetic sequences having codons different than the native gene). Allelic variations or naturally occurring mutational events do not give rise to a heterologous region of DNA as defined herein.

A “gene” includes all the DNA sequences associated with the promoter and coding region and non-coding region such as introns and 5′ and 3′ non-coding sequences and enhancer elements.

A “coding region” is an in-frame sequence of codons from the start codon, normally ATG, to the stop codon TAA, and which may or may not include introns.

A “coding sequence” is an in-frame sequence of codons that correspond to or encode a protein or peptide sequence. Two coding sequences correspond to each other if the sequences or their complementary sequences encode the same amino acid sequences. A coding sequence in association with appropriate regulatory sequences may be transcribed and translated into a polypeptide in vivo. A polyadenylation signal and transcription termination sequence will usually be located 3′ to the coding sequence.

A “promoter sequence” is a DNA regulatory region capable of binding RNA polymerase in a cell and initiating transcription of a downstream (3′direction) coding sequence. A coding sequence is “under the control” of the promoter sequence in a cell when RNA polymerase which binds the promoter sequence transcribes the coding sequence into mRNA, which is then in turn translated into the protein encoded by the coding sequence.

For the purposes of the present invention, the promoter sequence is bounded at its 3′ terminus by the translation start codon of a coding sequence, and extends upstream to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes, prokaryotic promoters contain Shine-Delgarno sequences in addition to the −10 and −35 consensus sequences.

A cell has been “transformed” by exogenous DNA when such exogenous DNA has been introduced inside the cell wall. Exogenous DNA may or may not be integrated (covalently linked) to chromosomal DNA making up the genome of the cell. In prokaryotes and yeast, for example, the exogenous DNA may be maintained on an episomal element such as a plasmid. With respect to eukaryotic cells, a stably transformed cell is one in which the exogenous DNA is inherited by daughter cells through chromosome replication. This stability is demonstrated by the ability of the eukaryotic cell to establish cell lines or clones comprised of a population of daughter cells containing the exogenous DNA.

“Integration” of the DNA may be effected using non-homologous recombination following mass transfer of DNA into the cells using microinjection, biolistics, electroporation or lipofection. Alternative methods such as homologous recombination, and or restriction enzyme mediated integration (REMI) or transposons are also encompassed, and may be considered to be improved integration methods.

A “clone” is a population of cells derived from a single cell or common ancestor by mitosis.

“Cell,” “host cell,” “cell line,” and “cell culture” are used interchangeably herewith and all such terms should be understood to include progeny. A “cell line” is a clone of a primary cell that is capable of stable growth in vitro for many generations. Thus the words “transformants” and “transformed cells” include the primary subject cell and cultures derived therefrom, without regard for the number of times the cultures have been passaged. It should also be understood that all progeny might not be precisely identical in DNA content, due to deliberate or inadvertent mutations.

Vectors are used to introduce a foreign substance, such as DNA, RNA or protein, into an organism. Typical vectors include recombinant viruses (for DNA) and liposomes (for protein). A “DNA cloning vector” is an autonomously replicating DNA molecule,” such as plasmid, phage or cosmid. Typically the DNA cloning vector comprises one or a small number of restriction endonuclease recognition sites, at which such DNA sequences may be cut in a determinable fashion without loss of an essential biological function of the vector, and into which a DNA fragment may be spliced in order to bring about its replication and cloning. The cloning vector may also comprise a marker suitable for use in the identification of cells transformed with the cloning vector.

An “expression vector” is similar to a DNA cloning vector, but contains regulatory sequences which are able to direct protein synthesis by an appropriate host cell. This usually means a promoter to bind RNA polymerase and initiate transcription of mRNA, as well as ribosome binding sites and initiation signals to direct translation of the mRNA into a polypeptide. Incorporation of a DNA sequence into an expression vector at the proper site and in correct reading frame, followed by transformation of an appropriate host cell by the vector, enables the production of mRNA corresponding to the DNA sequence, and usually of a protein encoded by the DNA sequence.

“Plasmids” are DNA molecules that are capable of replicating within a host cell, either extrachromosomally or as part of the host cell chromosome(s), and are designated by a lower case “p” preceded and/or followed by capital letters and/or numbers. The starting plasmids herein are commercially available, are publicly available on an unrestricted basis, or can be constructed from such available plasmids by methods disclosed herein and/or in accordance with published procedures. In certain instances, as will be apparent to the ordinarily skilled worker, other plasmids known in the art may be used interchangeably with plasmids described herein.

“Control sequences” refers to DNA sequences necessary for the expression of an operably linked nucleotide coding sequence in a particular host cell. The control sequences suitable for expression in prokaryotes, for example, include origins of replication, promoters, ribosome binding sites, and transcription termination sites. The control sequences that are suitable for expression in eukaryotes, for example, include origins of replication, promoters, ribosome binding sites, polyadenylation signals, and enhancers.

An “exogenous” element is one that is foreign to the host cell, or is homologous to the host cell but in a position within the host cell in which the element is ordinarily not found.

“Digestion” of DNA refers to the catalytic cleavage of DNA with an enzyme that acts only at certain locations in the DNA. Such enzymes are called restriction enzymes or restriction endonucleases, and the sites within DNA where such enzymes cleave are called restriction sites. If there are multiple restriction sites within the DNA, digestion will produce two or more linearized DNA fragments (restriction fragments). The various restriction enzymes used herein are commercially available, and their reaction conditions, cofactors, and other requirements as established by the enzyme manufacturers are used. Restriction enzymes are commonly designated by abbreviations composed of a capital letter followed by other letters representing the microorganism from which each restriction enzyme originally was obtained and then a number designating the particular enzyme. In general, about 1 μg of DNA is digested with about 1-2 units of enzyme in about 20 μl of buffer solution. Appropriate buffers and substrate amounts for particular restriction enzymes are specified by the manufacturer, and/or are well known in the art.

“Recovery” or “isolation” of a given fragment of DNA from a restriction digest typically is accomplished by separating the digestion products, which are referred to as “restriction fragments,” on a polyacrylamide or agarose gel by electrophoresis, identifying the fragment of interest on the basis of its mobility relative to that of marker DNA fragments of known molecular weight, excising the portion of the gel that contains the desired fragment, and separating the DNA from the gel, for example by electroelution.

“Ligation” refers to the process of forming phosphodiester bonds between two double-stranded DNA fragments. Unless otherwise specified, ligation is accomplished using known buffers and conditions with 10 units of T4 DNA ligase per 0.5 μg of approximately equimolar amounts of the DNA fragments to be ligated.

“Oligonucleotides” are short-length, single- or double-stranded polydeoxynucleotides that are chemically synthesized by known methods (involving, for example, triester, phosphoramidite, or phosphonate chemistry), such as described by Engels et al., Agnew. Chem. Int. Ed. Engl. 28:716-734 (1989). They are then purified, for example, by polyacrylamide gel electrophoresis.

“Polymerase chain reaction,” or “PCR,” as used herein generally refers to a method for amplification of a desired nucleotide sequence in vitro, as described in U.S. Pat. No. 4,683,195. In general, the PCR method involves repeated cycles of primer extension synthesis, using two oligonucleotide primers capable of hybridizing preferentially to a template nucleic acid. Typically, the primers used in the PCR method will be complementary to nucleotide sequences within the template at both ends of or flanking the nucleotide sequence to be amplified, although primers complementary to the nucleotide sequence to be amplified also may be used. See Wang et al., in PCR Protocols, pp.70-75 (Academic Press, 1990); Ochman et al., in PCR Protocols, pp. 219-227; Triglia, et al., Nuc. Acids Res. 16:8186 (1988).

“PCR cloning” refers to the use of the PCR method to amplify a specific desired nucleotide sequence that is present amongst the nucleic acids from a suitable cell or tissue source, including total genomic DNA and cDNA transcribed from total cellular RNA. See Frohman et al., Proc. Nat. Acad. Sci. USA 85:8998-9002 (1988); Saiki et al., Science. 239:487-492 (1988); Mullis et al., Meth. Enzymol. 155:335-350 (1987).

“zBMP2 promoter” refers to a promoter encoded by the nucleotide sequence set forth in SEQ ID NO.:1. “zSMAD promoter” refers to a promoter encoded by the nucleotide sequence set forth in SEQ ID NO.:8. “goosecoid promoter” refers to a promoter encoded by the nucleotide sequence set forth in SEQ ID NO:60. “Blocker molecule” refers to either antisense RNA, dsRNA, sense RNA or DNA that preferably encodes BMP2, GSC, HoxCG1 or HoxCG3 and includes the sequences shown in SEQ ID NO:13, SEQ ID NO:20, SEQ ID NO:23, SEQ ID NO:24, and SEQ ID NO:61. However, it will be appreciated by those skilled in the art that any nucleic acid molecule capable of disrupting gametogenesis or embryogenesis is encompassed. Accordingly, the terms “blocker molecule RNA” and “blocker molecule DNA” as used herein are interchangeable depending upon whether it is a species of RNA or DNA, that is being addressed. “HoxCG” refers to genes HoxCG1 and HoxCG3 isolated from Pacific oyster encoded by the nucleotide sequences set forth in SEQ ID NO.:23 and SEQ ID NO:24, respectively. Sequence variants of zBMP2 promoter, SMAD promoter, goosecoid promoter and HoxCG blocker molecules may be made synthetically, for example, by site-directed or PCR mutagenesis, or may exist naturally, as in the case of allelic forms and other naturally occurring variants of the nucleotide sequences set forth in SEQ ID NO.:1, SEQ ID NO:8, SEQ ID NO:60, SEQ ID NO:23, and SEQ ID NO:24, respectively, that may occur in fish and other animal species.

zBMP2 promoter, SMAD promoter, goosecoid promoter HoxCG, and blocker molecule nucleotide sequence variants are included within the scope of the invention, provided that they are functionally active. As used herein, “functionally active” and “functional activity” with reference to zBMP2 promoter, SMAD promoter, goosecoid promoter and HoxCG means that the zBMP2 promoter, SMAD promoter, goosecoid promoter and HoxCG variants are able to function in a similar way to naturally occurring zBMP2 promoter, SMAD promoter, goosecoid promoter and HoxCG. With reference to the blocker molecule “functionally active” and “functional activity” means that the blocker molecule variants are capable of disrupting gametogenesis or embyrogenesis in an animal. Therefore, zBMP2 promoter, SMAD promoter, goosecoid promoter HoxCG and blocker molecule nucleotide sequence variants generally will share at least about 75%, preferably greater than 80% and more preferably greater than 90%, sequence identity with the nucleotide sequences set forth in SEQ ID NO.:1, SEQ ID NO:8, SEQ ID NO:60, SEQ ID NO:23, and SEQ ID NO:24 respectively, after aligning the sequences to provide for maximum homology, as determined, for example, by the Fitch et al., Proc. Nat. Acad. Sci. USA 80:1382-1386 (1983), version of the algorithm described by Needleman et al., J. Mol. Biol. 48:443-453 (1970).

Nucleotide sequence variants of zBMP2 promoter, SMAD promoter, goosecoid promoter HoxCG and blocker molecule are prepared by introducing appropriate nucleotide changes into zBMP2 promoter, SMAD promoter, goosecoid promoter, HoxCG and blocker molecule DNA, or by in vitro synthesis. Such variants include deletions from, or insertions or substitutions of, nucleotides within the zBMP2 promoter, SMAD promoter, goosecoid promoter, HoxCG or blocker molecule nucleotide sequences set forth in SEQ ID NO.:1, SEQ ID NO:8, SEQ ID NO: 60, SEQ ID NO:23, and SEQ ID NO:24. Any combination of deletion, insertion, and substitution may be made to arrive at a nucleotide sequence variant of zBMP2 promoter, SMAD promoter, goosecoid promoter HoxCG or blocker molecule provided that such variants possess the desired characteristics described herein. Changes that are made in the nucleotide sequence set forth in SEQ ID NO.:1, SEQ ID NO:8, SEQ ID NO:60, SEQ ID NO:23, and SEQ ID NO:24, respectively, to arrive at nucleotide sequence variants of zBMP2 promoter, SMAD promoter, goosecoid promoter and HoxCG blocker molecules also may result in further modifications of the zBMP2 promoter, SMAD promoter, goosecoid promoter, HoxCG or blocker molecule upon their activation in host cells.

There are two principal variables in the construction of nucleotide sequence variants of zBMP2 promoter, SMAD promoter, goosecoid promoter, HoxCG and blocker molecule nucleic acid: the location of the mutation site and the nature of the mutation. These are variants from the nucleotide sequences set forth in SEQ ID NO.:1, SEQ ID NO:8, SEQ ID NO 60, SEQ ID NO:23, and SEQ ID NO:24 and may represent naturally occurring allelic forms of zBMP2 promoter, SMAD promoter, goosecoid promoter, HoxCG and blocker molecule or predetermined mutant forms of zBMP2 promoter, SMAD promoter, goosecoid promoter, HoxCG and blocker molecule made by mutating zBMP2 promoter, SMAD promoter, goosecoid promoter, HoxCG or blocker molecule DNA, either to arrive at an allele or a variant not found in nature. In general, the location and nature of the mutation chosen will depend upon the zBMP2 promoter, SMAD promoter, goosecoid promoter, HoxCG or blocker molecule characteristic to be modified.

Nucleotide sequence deletions generally range from about 1 to 30 nucleotides, more preferably about 1 to 10 nucleotides, and are typically contiguous.

Nucleotide sequence insertions include fusions ranging in length from one nucleotide to hundreds of nucleotides, as well as intrasequence insertions of single or multiple nucleotides. Intrasequence insertions (i.e., insertions made within the nucleotide sequences set forth in SEQ ID NO:1, SEQ ID NO:8, SEQ ID NO:60, SEQ ID NO:23, and SEQ ID NO:24) may range generally from about 1 to 10 nucleotides, more preferably 1 to 5, most preferably 1 to 3.

The third group of variants are those in which nucleotides in the nucleotide sequences set forth in SEQ ID NO.:1, SEQ ID NO:8, SEQ ID NO:60, SEQ ID NO.23, and SEQ ID NO:24 have been substituted with other nucleotides. Preferably one to four, more preferably one to three, even more preferably one to two, and most preferably only one nucleotide has been removed and a different nucleotide inserted in its place. The sites of greatest interest for making such substitutions are those sites that are likely to be important to the functional activity of the zBMP2 promoter, SMAD promoter, goosecoid promoter, HoxCG or blocker molecule.

zBMP2 promoter, SMAD promoter, goosecoid promoter, HoxCG and blocker molecule DNA is obtained from cDNA or genomic DNA libraries, or by in vitro synthesis. Identification of zBMP2 promoter, SMAD promoter, goosecoid promoter, HoxCG or blocker molecule DNA within a cDNA or a genomic DNA library, or in some other mixture of various DNAs, is conveniently accomplished by the use of an oligonucleotide hybridization probe labelled with a detectable moiety, such as a radioisotope. See Keller et al., DNA Probes, pp.149-213 (Stockton Press, 1989). To identify DNA encoding zBMP2 promoter, SMAD promoter, goosecoid promoter, HoxCG or blocker molecule DNA, the nucleotide sequence of the hybridization probe is preferably selected so that the hybridization probe is capable of hybridizing preferentially to DNA encoding homologues of the equivalent zBMP2 promoter, SMAD promoter, goosecoid promoter, HoxCG or blocker molecule DNA in other species, or variants or derivatives thereof as described herein, under the hybridization conditions chosen. Another method for obtaining zBMP2 promoter, SMAD promoter, goosecoid promoter, HoxCG or blocker molecule is chemical synthesis using one of the methods described, for example, by Engels et al., Agnew. Chem. Int. Ed. Engl. 28:716-734 (1989).

If the entire nucleotide coding sequence for zBMP2 promoter, SMAD promoter, goosecoid promoter, HoxCG or blocker molecule is not obtained in a single cDNA, genomic DNA, or other DNA, as determined, for example, by DNA sequencing or restriction endonuclease analysis, then appropriate DNA fragments (e.g., restriction fragments or PCR amplification products) may be recovered from several DNA's, and covalently joined to one another to construct the entire coding sequence. The preferred means of covalently joining DNA fragments is by ligation using a DNA ligase enzyme, such as T4 DNA ligase.

“Isolated” zBMP2 promoter, SMAD promoter, goosecoid promoter, HoxCG or blocker molecule nucleic acid is zBMP2 promoter, SMAD promoter, goosecoid promoter, HoxCG or blocker molecule nucleic acid that is identified and separated from (or otherwise substantially free from), contaminant nucleic acid encoding other polypeptides. The isolated zBMP2 promoter, SMAD promoter, goosecoid promoter, HoxCG or blocker molecule can be incorporated into a plasmid or expression vector, or can be labeled for probe purposes, using a label as described further herein in the discussion of assays and nucleic acid hybridization methods.

It will be appreciated that if the desired result of the present invention is sterilized adult feral animals then the blocker molecules may be expressed in vitro, isolated, purified, and then delivered to specific organisms. The mode of delivery may be any known procedure including injection and ingestion. Moreover, constructs of the present invention which are capable of expressing blocker molecules may also be delivered to adult feral animals by viral vectors like adenovirus. Isolated zBMP2 promoter, SMAD promoter and goosecoid promoter nucleic acid is also used to control the expression of other desired genes or blocker molecules in vivo. Indeed, the zBMP2 promoter, SMAD promoter and goosecoid promoter may be used in any vector, or construct where the expression of a gene, cDNA, or coding sequence is desirably controlled to be at a particular spatio-temporal point during embyrogenesis. It will be appreciated that while the zBMP2 promoter and SMAD promoter are particularly useful in controlling the expression of nucleic acids in fish, they are equally useful in other organisms. In various embodiments of the invention, host cells are transformed or transfected with recombinant DNA molecules comprising an isolated zBMP2 promoter or SMAD promoter DNA or goosecoid promoter operably linked to a desired nucleic acid molecule, wherein the expression of the desired molecule is directly or indirectly under the control of the zBMP2 promoter or SMAD promoter or goosecoid promoter.

Isolated HoxCG nucleic acid is also used to produce HoxCG by recombinant DNA and recombinant cell culture methods. In various embodiments of the invention, host cells are transformed or transfected with recombinant DNA molecules comprising an isolated HoxCG DNA, to obtain expression of the HoxCG DNA and thus the production of HoxCG in large quantities. DNA encoding amino acid sequence variants of HoxCG is prepared by a variety of methods known in the art. These methods include, but are not limited to, isolation from a natural source (in the case of naturally occurring amino acid sequence variants of HoxCG), or preparation by site-directed or oligonucleotide-mediated mutagenesis, PCR mutagenesis, and cassette mutagenesis of DNA encoding a variant or a non-variant form of HoxCG.

Site-directed mutagenesis is a preferred method for preparing substitution, deletion, and insertion variants of HoxCG DNA, or other DNA such as the zBMP2 promoter, SMAD promoter, and blocker molecule DNA. This technique is well known in the art; see Zoller et al., Meth. Enz. 100:4668-500 (1983); Zoller, et al., Meth. Enz. 154:329-350 (1987); Carter, Meth. Enz. 154:382-403 (1987); Horwitz et al., Meth. Enz. 185:599-611 (1990), and has been used to produce amino acid sequence variants of trypsin and T4 lysozyme, which variants have certain desired functional properties. Perry et al., Science 226:555-557 (1984); Craik et al., Science 228:291-297 (1985).

Briefly, in carrying out site-directed mutagenesis of zBMP2 promoter, SMAD promoter, goosecoid promoter, HoxCG and blocker molecule DNA, the zBMP2 promoter, SMAD promoter, goosecoid promoter, HoxCG and blocker molecule DNA is altered by first hybridizing an oligonucleotide encoding the desired mutation to a single strand of zBMP2 promoter, SMAD promoter, goosecoid promoter, HoxCG and blocker molecule DNA. After hybridization, a DNA polymerase is used to synthesize an entire second strand, using the hybridized oligonucleotide as a primer, and using the single strand of zBMP2 promoter, SMAD promoter, goosecoid promoter, HoxCG and blocker molecule DNA as a template. Thus the oligonucleotide encoding the desired mutation is incorporated into the resulting double-stranded DNA.

Oligonucleotides for use as hybridization probes or primers may be prepared by any suitable method, such as purification of a naturally occurring DNA or in vitro synthesis. For example, oligonucleotides are readily synthesized using various techniques in such as those described by Narang et al., Meth. Enzymol. 68:90-98 (1979); Brown et al., Meth. Enzymol. 68:109-151 (1979); Caruther et al., Meth. Enzymol. 154:287-313 (1985). The general approach to selecting a suitable hybridization probe or primer is well known. Keller et al., DNA Probes, pp.11-18 (Stockton Press, 1989). Typically, the hybridization probe or primer will contain 10-25 or more nucleotides, and will include at least 5 nucleotides on either side of the sequence encoding the desired mutation so as to ensure that the oligonucleotide will hybridize preferentially to the single-stranded DNA template molecule.

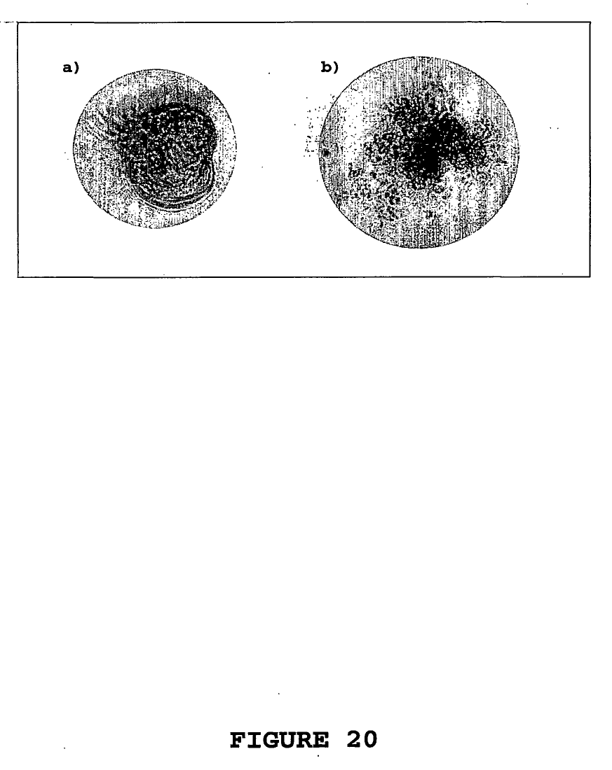

Multiple mutations are introduced into HoxCG DNA to produce amino acid sequence variants of HoxCG comprising several or a combination of insertions, deletions, or substitutions of amino acid residues as compared to the amino acid sequences set forth in FIG. 20. If the sites to be mutated are located close together, the mutations may be introduced simultaneously using a single oligonucleotide that encodes all of the desired mutations. If, however, the sites to be mutated are located some distance from each other (separated by more than about ten nucleotides), it is more difficult to generate a single oligonucleotide that encodes all of the desired changes. Instead, one of two alternative methods may be employed.

In the first method, a separate oligonucleotide is generated for each desired mutation. The oligonucleotides are then simultaneously annealed to the single-stranded template DNA, and the second strand of DNA that is synthesized from the template will encode all of the desired amino acid substitutions.

The alternative method involves two or more rounds of mutagenesis to produce the desired mutant. The first round is as described for introducing a single mutation: a single strand of a previously prepared HoxCG DNA is used as a template, an oligonucleotide encoding the first desired mutation is annealed to this template, and a heteroduplex DNA molecule is then generated. The second round of mutagenesis utilizes the mutated DNA produced in the first round of mutagenesis as the template. Thus this template already contains one or more mutations. The oligonucleotide encoding the additional desired amino acid substitution(s) is then annealed to this template, and the resulting strand of DNA now encodes mutations from both the first and second rounds of mutagenesis. This resultant DNA can be used as a template in a third round of mutagenesis, and so on.

PCR mutagenesis is also suitable for making nucleotide sequence variants of zBMP2 promoter, SMAD promoter, goosecoid promoter, HoxCG and blocker molecule. Higuchi, in PCR Protocols, pp.177-183 (Academic Press, 1990); Vallette et al., Nuc. Acids Res. 17:723-733 (1989). Briefly, when small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template. For introduction of a mutation into a plasmid DNA, for example, one of the primers is designed to overlap the position of the mutation and to contain the mutation; the sequence of the other primer must be identical to a nucleotide sequence within the opposite strand of the plasmid DNA, but this sequence can be located anywhere along the plasmid DNA. It is preferred, however, that the sequence of the second primer is located within 200 nucleotides from that of the first, such that in the end the entire amplified region of DNA bounded by the primers can be easily sequenced. PCR amplification using a primer pair like the one just described results in a population of DNA fragments that differ at the position of the mutation specified by the primer, and possibly at other positions, as template copying is somewhat error-prone. See Wagner et al., in PCR Topics, pp.69-71 (Springer-Verlag, 1991).

If the ratio of template to product amplified DNA is extremely low, the majority of product DNA fragments incorporate the desired mutation(s). This product DNA is used to replace the corresponding region in the plasmid that served as PCR template using standard recombinant DNA methods. Mutations at separate positions can be introduced simultaneously by either using a mutant second primer, or performing a second PCR with different mutant primers and ligating the two resulting PCR fragments simultaneously to the plasmid fragment in a three (or more)-part ligation.

Another method for preparing variants, cassette mutagenesis, is based on the technique described by Wells et al., Gene, 34:315-323 (1985). The starting material is the plasmid (or other vector) comprising the zBMP2 promoter, SMAD promoter, goosecoid promoter, HoxCG or blocker molecule DNA to be mutated. The codon(s) in the zBMP2 promoter, SMAD promoter, goosecoid promoter, HoxCG or blocker molecule DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the zBMP2 promoter, SMAD promoter, goosecoid promoter, HoxCG and blocker molecule DNA. The plasmid DNA is cut at these sites to linearize it. A double-stranded oligonucleotide encoding the sequence of the DNA between the restriction sites but containing the desired mutation(s) is synthesized using standard procedures, wherein the two strands of the oligonucleotide are synthesized separately and then hybridized together using standard techniques. This double-stranded oligonucleotide is referred to as the cassette. This cassette is designed to have 5′ and 3′ ends that are compatible with the ends of the linearized plasmid, such that it can be directly ligated to the plasmid. This plasmid now contains the mutated zBMP2 promoter, SMAD promoter, goosecoid promoter, HoxCG, or blocker molecule DNA sequence.

zBMP2 promoter, SMAD promoter, goosecoid promoter, HoxCG, and blocker molecule DNA, whether cDNA or genomic DNA or a product of in vitro synthesis, is ligated into a replicable vector for further cloning or for expression. “Vectors” are plasmids and other DNA's that are capable of replicating autonomously within a host cell, and as such, are useful for performing two functions in conjunction with compatible host cells (a vector-host system). One function is to facilitate the cloning of the nucleic acid that encodes the zBMP2 promoter, SMAD promoter, goosecoid promoter, HoxCG, and blocker molecule, i.e., to produce usable quantities of the nucleic acid. The other function is to direct the expression of HoxCG. One or both of these functions are performed by the vector-host system. The vectors will contain different components depending upon the function they are to perform as well as the host cell with which they are to be used for cloning or expression.

To produce HoxCG, an expression vector will contain nucleic acid that encodes HoxCG as described above. The HoxCG of this invention may be expressed directly in recombinant cell culture, or as a fusion with a heterologous polypeptide, preferably a signal sequence or other polypeptide having a specific cleavage site at the junction between the heterologous polypeptide and the HoxCG.

In one example of recombinant host cell expression, cells are transfected with an expression vector comprising HoxCG DNA and the HoxCG encoded thereby is recovered from the culture medium in which the recombinant host cells are grown. But the expression vectors and methods disclosed herein are suitable for use over a wide range of prokaryotic and eukaryotic organisms.

Prokaryotes may be used for the initial cloning of DNA's and the construction of the vectors useful in the invention. However, prokaryotes may also be used for expression of mRNA or protein encoded by HoxCG. Polypeptides that are produced inprokaryotic host cells typically will be non-glycosylated.

Plasmid or viral vectors containing replication origins and other control sequences that are derived from species compatible with the host cell are used in connection with prokaryotic host cells, for cloning or expression of an isolated DNA. For example, E. coli typically is transformed using pBR322 a plasmid derived from an E. coli species. Bolivar et al., Gene 2:95-113 (1987). PBR322 contains genes for ampicillin and tetracycline resistance so that cells transformed by the plasmid can easily be identified or selected. For it to serve as an expression vector, the pBR322 plasmid, or other plasmid or viral vector, must also contain, or be modified to contain, a promoter that functions in the host cell to provide messenger RNA (mRNA) transcripts of a DNA inserted downstream of the promoter. Rangagwala et al., Bio/Technology 9:477-479 (1991).

In addition to prokaryotes, eukaryotic microbes, such as yeast, may also be used as hosts for the cloning or expression of DNA's useful in the invention. Saccharomyces cerevisiae, or common baker's yeast, is the most commonly used eukaryotic microorganism. Plasmids useful for cloning or expression in yeast cells of a desired DNA are well known, as are various promoters that function in yeast cells to produce mRNA transcripts.

Furthermore, cells derived from multicellular organisms also may be used as hosts for the cloning or expression of DNA's useful in the invention. Mammalian cells are most commonly used, and the procedures for maintaining or propagating such cells in vitro, which procedures are commonly referred to as tissue culture, are well known. Kruse & Patterson, eds., Tissue Culture (Academic Press, 1977). Examples of useful mammalian cells are human cell lines such as 293, HeLa, and WI-38, monkey cell lines such as COS-7 and VERO, and hamster cell lines such as BHK-21 and CHO, all of which are publicly available from the American Type Culture Collection (ATCC), Rockville, Md. 20852 USA.

Expression vectors, unlike cloning vectors, should contain a promoter that is recognized by the host organism and is operably linked to the HoxCG nucleic acid. Promoters are untranslated sequences that are located upstream from the start codon of a gene and that control transcription of the gene (that is, the synthesis of mRNA). Promoters typically fall into two classes, inducible and constitutive. Inducible promoters are promoters that initiate high level transcription of the DNA under their control in response to some change in culture conditions, for example, the presence or absence of a nutrient or a change in temperature.

A large number of promoters are known, that may be operably linked to HoxCG DNA to achieve expression of HoxCG in a host cell. This is not to say that the promoter associated with naturally occurring HoxCG DNA is not usable. However, heterologous promoters generally will result in greater transcription and higher yields of expressed HoxCG.

Promoters suitable for use with prokaryotic hosts include the β-lactamase and lactose promoters, Goeddel et al., Nature 281:544-548 (1979), tryptophan (trp) promoter, Goeddel et al., Nuc. Acids Res. 8:4057-4074 (1980), and hybrid promoters such as the tac promoter, deBoer et al., Proc. Natl. Acad. Sci. USA 80:21-25 (1983). However, other known bacterial promoters are suitable. Their nucleotide sequences have been published, Siebenlist et al., Cell 20:269-281 (1980), thereby enabling a skilled worker operably to ligate them to DNA encoding HoxCG using linkers or adaptors to supply any required restriction sites. See Wu et al., Meth. Enz. 152:343-349 (1987).

Suitable promoters for use with yeast hosts include the promoters for 3-phosphoglycerate kinase, Hitzeman et al., J. Biol. Chem. 255:12073-12080 (1980); Kingsman et al., Meth. Enz. 185:329-341 (1990), or other glycolytic enzymes such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Dodson et al., Nuc. Acids res. 10:2625-2637 (1982); Emr, Meth. Enz. 185:231-279 (1990).

Expression vectors useful in mammalian cells typically include a promoter derived from a virus. For example, promoters derived from polyoma virus, adenovirus, cytomegalovirus (CMV), and simian virus 40 (SV40) are commonly used. Further, it is also possible, and often desirable, to utilize promoter or other control sequences associated with a naturally occurring DNA that encodes HoxCG, provided that such control sequences are functional in the particular host cell used for recombinant DNA expression. In particular, in the present invention it may be desirable to utilize the zBMP2 promoter or SMAD promoter or goosecoid promoter such that a spatio-temporal expression of the HoxCG occurs.

Other control sequences that are desirable in an expression vector in addition to a promoter are a ribosome-binding site, and in the case of an expression vector used with eukaryotic host cells, an enhancer. Enhancers are cis-acting elements of DNA, usually about from 10-300 bp, that act on a promoter to increase the level of transcription. Many enhancer sequences are now known from mammalian genes (for example, the genes for globin, elastase, albumin, α-fetoprotein and insulin). Typically, however, the enhancer used will be one from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See Kriegler, Meth. Enz. 185:512-527 (1990).

Expression vectors may also contain sequences necessary for the termination of transcription and for stabilizing the messenger RNA (mRNA). Balbas et al., Meth. Enz. 185:14-37 (1990); Levinson, Meth. Enz. 185:485-511 (1990). In the case of expression vectors used with eukaryotic host cells, such transcription termination sequences may be obtained from the untranslated regions of eukaryotic or viral DNA's or cDNAs. These regions contain polyadenylation sites as well as transcription termination sites. Birnsteil et al., Cell 41:349-359 (1985).

In general, control sequences are DNA sequences necessary for the expression of an operably linked coding sequence in a particular host cell. “Expression” refers to transcription and/or translation. “Operably linked” refers to the covalent joining of two or more DNA sequences, by means of enzymatic ligation or otherwise, in a configuration relative to one another such that the normal function of the sequences can be performed. For example, DNA for a pre-sequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading frame. Linking is accomplished by ligation at convenient restriction sites. If such sites do not exist, then synthetic oligonucleotide adaptors or linkers are used, in conjunction with standard recombinant DNA methods.

Expression and cloning vectors also will contain a sequence that enables the vector to replicate in one or more selected host cells. Generally, in cloning vectors this sequence is one that enables the vector to replicate independently of the host chromosome(s), and includes origins of replication or autonomously replicating sequences. Such sequences are well known for a variety of bacteria, yeast, and viruses. The origin of replication from the plasmid pBR322 is suitable for most gram-negative bacteria, the 2 μ plasmid origin is suitable for yeast, and various viral origins (for example, from SV40, polyoma, or adenovirus) are useful for cloning vectors in mammalian cells. Most expression vectors are “shuttle” vectors, i.e. they are capable of replication in at least one class of organisms but can be transfected into another organism for expression. For example, a vector may be cloned in E. coli and then the same vector is transfected into yeast or mammalian cells for expression even though it is not capable of replicating independently of the host cell chromosome.

The expression vector may also include an amplifiable gene, such as that comprising the coding sequence for dihydrofolate reductase (DHFR). Cells containing an expression vector that includes a DHFR gene may be cultured in the presence of methotrexate, a competitive antagonist of DHFR. This leads to the synthesis of multiple copies of the DHFR gene and, concomitantly, multiple copies of other DNA sequences comprising the expression vector, Ringold et al., J. Mol. Apl. Genet. 1:165-175 (1981), such as a DNA sequence encoding HoxCG. In that manner, the level of HoxCG produced by the cells may be increased.

DHFR protein encoded by the expression vector also may be used as a selectable marker of successful transfection. For example, if the host cell prior to transformation is lacking in DHFR activity, successful transformation by an expression vector comprising DNA sequences encoding HoxCG and DHFR protein can be determined by cell growth in medium containing methotrexate. Also, mammalian cells transformed by an expression vector comprising DNA sequences encoding HoxCG, DHFR protein, and aminoglycoside 3′ phosphotransferase (APH) can be determined by cell growth in medium containing an aminoglycoside antibiotic such as kanamycin or neomycin. Because eukaryotic cells do not normally express an endogenous APH activity, genes encoding APH protein, commonly referred to as neo^{r} genes, may be used as dominant selectable markers in a wide range of eukaryotic host cells, by which cells transfected by the vector can easily be identified or selected. Jiminez et al., Nature, 287:869-871 (1980); Colbere-Garapin et al., J. Mol. Biol. 150:1-14 (1981); Okayama & Berg, Mol. Cell. Biol., 3:280-289 (1983).

Many other selectable markers are known that may be used for identifying and isolating recombinant host cells that express HoxCG. For example, a suitable selection marker for use in yeast is the trp1 gene present in the yeast plasmid YRp7. Stinchcomb et al., Nature 282:39-43 (1979); Kingsman et al., Gene 7:141-152 (1979); Tschemper et al., Gene 10:157-166 (1980). The trp1 gene provides a selection marker for a mutant strain of yeast lacking the ability to grow in tryptophan, for example, ATCC No. 44076 or PEP4-1 (available from the American Type Culture Collection, Rockville, Md. 20852 USA). Jones, Genetics 85:12 (1977). The presence of the trp1 lesion in the yeast host cell genome then provides an effective environment for detecting transformation by growth in the absence of tryptophan. Similarly, Leu2-deficient yeast strains (ATCC Nos. 20622 or 38626) are complemented by known plasmids bearing the Leu2 gene.

Particularly useful in the invention are expression vectors that provide for the transient expression in mammalian cells of DNA encoding HoxCG. In general, transient expression involves the use of an expression vector that is able to efficiently replicate in a host cell, such that the host cell accumulates many copies of the expression vector and, in turn, synthesizes high levels of a desired polypeptide encoded by the expression vector. Transient expression systems, comprising a suitable expression vector and a host cell, allow for the convenient positive identification of polypeptides encoded by cloned DNA's, as well as for the rapid screening of such polypeptides for desired biological or physiological properties. Yang et al., Cell 47:3-10 (1986); Wong et al., Science 228:810-815 (1985); Lee et al., Proc. Nat Acad. Sci. USA 82:4360-4364 (1985). Thus, transient expression systems are particularly useful in the invention for expressing DNA's encoding amino acid sequence variants of HoxCG, to identify those variants which are functionally active.

Since it is often difficult to predict in advance the characteristics of an amino acid sequence variant of HoxCG, it will be appreciated that some screening of such variants will be needed to identify those that are functionally active. Such screening may be performed in vitro, using routine assays for receptor binding, or assays for cell proliferation, cell differentiation or cell viability, or using immunoassays with monoclonal antibodies that selectively bind to HoxCG that effect the functionally active HoxCG, such as a monoclonal antibody that selectively binds to the active site or receptor binding site of HoxCG.

As used herein, the terms “transformation” and “transfection” refer to the process of introducing a desired nucleic acid, such a plasmid or an expression vector, into a host cell. Various methods of transformation and transfection are available, depending on the nature of the host cell. In the case of E. coli cells, the most common methods involve treating the cells with aqueous solutions of calcium chloride and other salts. In the case of mammalian cells, the most common methods are transfection mediated by either calcium phosphate or DEAE-dextran, or electroporation. Sambrook et al., eds., Molecular Cloning, pp. 1.74-1.84 and 16.30-16.55 (Cold Spring Harbor Laboratory Press, 1989). Following transformation or transfection, the desired nucleic acid may integrate into the host cell genome, or may exist as an extrachromosomal element.

Host cells that are transformed or transfected with the above-described plasmids and expression vectors are cultured in conventional nutrient media modified as is appropriate for inducing promoters or selecting for drug resistance or some other selectable marker or phenotype. The culture conditions, such as temperature, pH, and the like, suitably are those previously used for culturing the host cell used for cloning or expression, as the case may be, and will be apparent to those skilled in the art.

Suitable host cells for cloning or expressing the vectors herein are prokaryotes, yeasts, and higher eukaryotes, including insect, oysters, lower vertebrate, and mammalian host cells. Suitable prokaryotes include eubacteria, such as Gram-negative or Gram-positive organisms, for example, E. coli, Bacillus species such as B. subtilis, Pseudomonas species such as P. aeruginosa, Salmonella typhimurium, or Serratia marcescans.

In addition to prokaryotes, eukaryotic microbes such as filamentous fungi or yeast are suitable hosts for zBMP2, HoxCG and blocker molecule-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe, Beach and Nurse, Nature 290:140-142 (1981), Pichia pastoris, Cregg et al., Bio/Technology 5:479-485 (1987); Sreekrishna, et al., Biochemistry 28:4117-4125 (1989), Neurospora crassa, Case, et al., Proc. Natl. Acad. Sci. USA 76:5259-5263 (1979), and Aspergillus hosts such as A. nidulans, Ballance et al., Biochem. Biophys. Res. Commun. 112:284-289 (1983); Tilburn et al., Gene 26:205-221 (1983); Yelton et al., Proc. Natl. Acad. Sci. USA 81:1470-1474 (1984), and A. niger, Kelly et al., EMBO J. 4:475-479 (1985).

Suitable host cells for the expression of HoxCG also are derived from multicellular organisms. Such host cells are capable of complex processing and glycosylation activities. In principle, any higher eukaryotic cell culture is useable, whether from vertebrate or invertebrate culture. It will be appreciated, however, that because of the species-, tissue-, and cell-specificity of glycosylation, Rademacher et al., Ann. Rev. Biochem. 57:785-838 (1988), the extent or pattern of glycosylation of HoxCG in a foreign host cell typically will differ from that of HoxCG obtained from a cell in which it is naturally expressed.

Examples of invertebrate cells include insect and plant cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori host cells have been identified. Luckow et al., Bio/Technology 6:47-55 (1988); Miller et al., in Genetic Engineering, vol. 8, pp.277-279 (Plenum Publishing, 0.1986); Maeda et al., Nature 315:592-594 (1985).

Plant cell cultures of cotton, corn, potato, soybean, petunia, tomato, and tobacco can be utilized as hosts. Typically, plant cells are transfected by incubation with certain strains of the bacterium Agrobacterium tumefaciens. During incubation of the plant cells with A. tumefaciens, the DNA is transferred into cells, such that they become transfected, and will, under appropriate conditions, express the introduced DNA. In addition, regulatory and signal sequences compatible with plant cells are available, such as the nopaline synthase promoter and polyadenylation signal sequences, and the ribulose biphosphate carboxylase promoter. Depicker et al., J. Mol. Appl. Gen. 1:561-573 (1982). Herrera-Estrella et al., Nature 310:115-120 (1984). In addition, DNA segments isolated from the upstream region of the T-DNA 780 gene are capable of activating or increasing transcription levels of plant-expressible genes in recombinant DNA-containing plant tissue. European Pat. Pub . . . . No. EP 321,196 (published Jun. 21, 1989).

However, interest has been greatest in vertebrate cells, and propagation of vertebrate cells in culture (tissue culture) has become a routine procedure in recent years. Kruse & Patterson, eds., Tissue Culture (Academic Press, 1973). Examples of useful mammalian host cells are the monkey kidney CV1 line transformed by SV40 (COS-7, ATCC CRL 1651); human embryonic kidney line 293 (or 293 cells subcloned for growth in suspension culture), Graham et al., J. Gen Virol. 36:59-72 (1977); baby hamster kidney cells (BHK, ATCC CCL 10); Chinese hamster ovary cells (including DHFR-deficient CHO cells, Urlaub et al., Proc. Natl. Acad. Sci. USA 77:421.6-4220 (1980); mouse sertoli cells (TM4, Mather, Biol. Reprod. 23:243-251 (1980); monkey kidney cells (CV1, ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Construction of suitable vectors containing the nucleotide sequence encoding HoxCG and appropriate control sequences employs standard recombinant DNA methods. DNA is cleaved into fragments, tailored, and ligated together in the form desired to generate the vectors required.

For analysis to confirm correct sequences in the vectors constructed, the vectors are analyzed by restriction digestion (to confirm the presence in the vector of predicted restriction endonuclease) and/or by sequencing by the dideoxy chain termination method of Sanger et al., Proc. Nat. Acad. Sci. USA 72:3918-3921 (1979).

The mammalian host cells used to produce the HoxCG of this invention may be cultured in a variety of media. Commercially available media such as Ham's F10 (Sigma), Minimal Essential Medium (MEM, Sigma), RPMI-1640 (Sigma), and Dulbecco's Modified Eagle's Medium (DMEM, Sigma) are suitable for culturing the host cells. In addition, any of the media described in Ham, et al., Meth. Enz. 58:44-93 (1979); Barnes et al., Anal. Biochem. 102:255-270 (1980); Bottenstein et al., Meth. Enz. 58:94-109 (1979); U.S. Pat. Nos. 4,560,655; 4,657,866; 4,767,704; or 4,927,762; or in PCT Pat. Pub. Nos. WO 90/03430 (published Apr. 5, 1990), may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

The host cells referred to in this disclosure encompass cells in culture in vitro as well as cells that are within a host animal, for example, as a result of transplantation or implantation.

It is further contemplated that the HoxCG of this invention may be produced by homologous recombination, for example, as described in PCT Pat. Pub. No. WO 91/06667 (published May 16, 1991). Briefly, this method involves transforming cells containing an endogenous gene encoding HoxCG with a homologous DNA, which homologous DNA comprises (1) an amplifiable gene, such as DHFR, and (2) at least one flanking sequence, having a length of at least about 150 base pairs, which is homologous with a nucleotide sequence in the cell genome that is within or in proximity to the gene encoding HoxCG. The transformation is carried out under conditions such that the homologous DNA integrates into the cell genome by recombination. Cells having integrated the homologous DNA then are subjected to conditions which select for amplification of the amplifiable gene, whereby the HoxCG gene amplified concomitantly. The resulting cells then are screened for production of desired amounts of HoxCG. Flanking sequences that are in proximity to a gene encoding HoxCG are readily identified, for example, by the method of genomic walking, using as a starting point the HoxCG nucleotide sequence set forth in SEQ ID NO.:23 and SEQ ID NO.:24. See Spoerel et al., Meth. Enz. 152:598-603 (1987).

Gene amplification and/or gene expression may be measured in a sample directly, for example, by conventional Southern blotting to quantitate DNA, or Northern blotting to quantitate mRNA, using an appropriately labeled oligonucleotide hybridization probe, based on the sequences provided herein. Various labels may be employed, most commonly radioisotopes, particularly ³²P. However, other techniques may also be employed, such as using biotin-modified nucleotides for introduction into a polynucleotide. The biotin then serves as the site for binding to avidin or antibodies, which may be labeled with a wide variety of labels, such as radioisotopes, fluorophores, chromophores, or the like. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of tissue sections and assay of cell culture or body fluids, to quantitate directly the expression of the gene product, HoxCG. With immunohistochemical staining techniques, a cell sample is prepared, typically by dehydration and fixation, followed by reaction with labeled antibodies specific for the gene product coupled, where the labels are usually visually detectable, such as enzymatic labels, fluorescent labels, luminescent labels, and the like. A particularly sensitive staining technique suitable for use in the present invention is described by Hsu et al., Am. J. Clin. Path., 75:734-738 (1980). Antibodies useful for immunohistochemical staining and/or assay of sample fluids may be either monoclonal or polyclonal. Conveniently, the antibodies may be prepared against a synthetic peptide based on the DNA sequences provided herein.

Throughout the description and claims of this specification, the word “comprise” and variations of the word, such as “comprising” and “comprises”, means “including but not limited to” and is not intended to exclude other additives, components, integers or steps.

The invention will now be further described by way of reference only to the following non-limiting examples. It should be understood, however, that the examples following are illustrative only, and should not be taken in any way as a restriction on the generality of the invention described above. Amino acid sequences referred to herein are given in standard single letter code.

### EXAMPLE 1

### Isolation of Stage-Specific Promoters for a Sterile Feral Construct

In order to identify a good candidate promoter and/or gene for the proposed construct, the applicant examined a number of animals, both vertebrate and invertebrate. The applicant finally decided on the well-studied model for fish, the zebrafish (Brachydanio rerio). This fish model was chosen as it is reasonably well characterized, and the fish are small and relatively easily breed and reared. Moreover, the zebrafish has a high degree of nucleotide and amino acid sequence homology to most other fish species studied, and as will be shown later, a reasonably high degree of sequence homology with other non-fish species. This degree of similarity can permit the identification of genes in other species by comparison with those of zebrafish. Accordingly, it was considered, that this model was most appropriate for locating and testing a promoter which may function across all species. At least it was a useful model for testing the broad “sterile feral construct” concept.

The applicant examined mutant screens in zebrafish for a target gene that was essential for a short period in larval development, but which had no adult functions. The applicant focused on 6 mutations that cause dorso-ventral patterning defects (Mullins et al 1996), and in particular on the swirl mutant, which exhibits severe dorsalization and the complete lack of ventral structures such as blood and pronephros. Swirl encodes the zebrafish homologue of BMP2 and was named zBMP2 (Kishimoto et al., 1997). In zebrafish the dorsalised swirl mutant phenotype is rescued by injection of zBMP2 mRNA at the single cell stage (Kishimoto et al., 1997), which indicates that the gene is essential only during early larval development and plays no maternal role. BMPs (Bone Morphogenetic Proteins) are a subfamily of the larger transforming growth factor beta (TGF-β) superfamily of signalling molecules that play a central role in establishing the early animal body plan and in organogenesis (Hogan, 1996).

The cDNA for the zBMP2 gene was obtained from M. Hammerschmidt (Max Plank Institute, Frieburg) as a 1,732 bp fragment subcloned into a plasmid designated pzBMP2b. This plasmid was transformed into XL-1 blue strain of E. coli according to the instructions of the supplier (Stratagene). A resulting positive clone carrying the plasmid was grown according to standard protocols, and the cDNA from the bacterial culture was isolated by standard procedures. After digestion with EcoRI, a 422 bp fragment spanning the 5′ untranslated region was isolated and labelled with ³²P. This was then used as a probe for a zebrafish genomic library.

The zebrafish genomic BAC library was purchased in the form of arrayed filter sets, from Genome Systems Inc (GSI), and screened using the labelled probe by standard hybridization techniques as described previously. Five positive clones (BMP-BAC5, BMP-BAC10, BMP-BAC15, BMP-BAC17, and BMP-BAC21) were then purchased from Genome Systems Inc (GSI). Preliminary sequencing of all five positive BAC clones using primers specific of the 5′-untranslated region of the cDNA revealed that the clones were identical to each other and to the region of the BMP2 cDNA. Two of the BAC clones (BMP-BAC5 and BMP-BAC10) were subcloned as HindIII fragments into pGEM-7ZF(+) by standard procedures. We obtained 6,915 bp of sequence from these clones which represented from −3879 to +3035 bp relative to the translation start site. The coding sequence obtained was identical to the zebrafish zBMP2 cDNA sequence previously described by Nikido et al. (1997) and Lee et al. (1998). This suggested that BAC 5 and 10, and perhaps the remaining three BAC clones, contained authentic zebrafish BMP2 genomic DNA. However, based on the genomic sequences we obtained, the previously designated start site, at 376 bp in the cDNA (Lee et al., 1998), lies in the second exon and the first exon is untranslated.

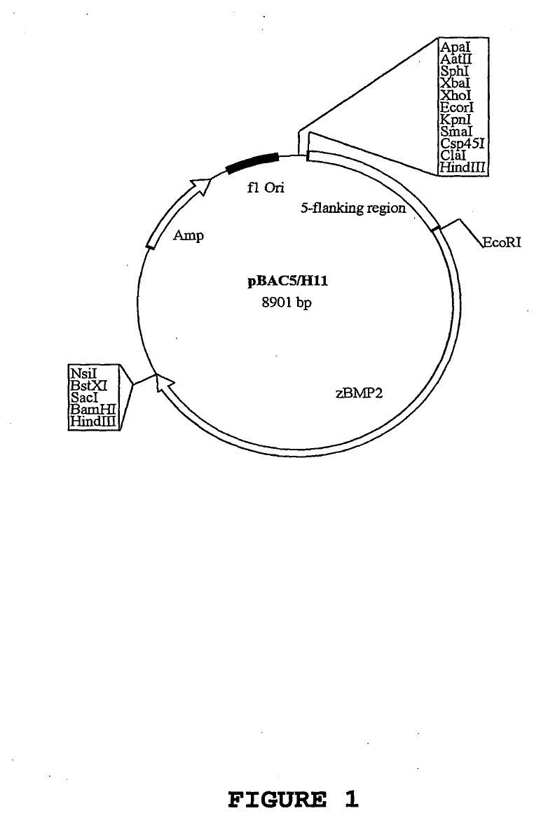

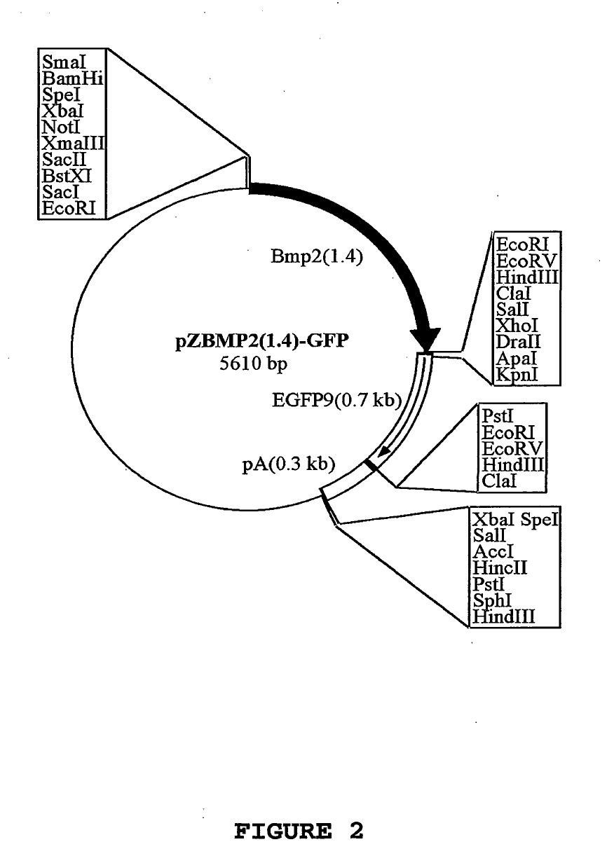

Further definition and isolation of the zBMP2 promoter was accomplished by sequencing these HindIII subclones to isolate candidate fragments which resided 5′ of the sequence homologous to the cDNA coding for zBMP2 gene. One of these subclones had a 5,901 bp insert that was positive for zBMP2 gene. FIG. 1 shows the resultant plasmid pBAC5/H11. The insert was also found to include a 1,414 bp region that was 5′ of the presumptive start codon of zBMP2, and which was considered to be a possible location of the zBMP2 promoter. A 1,414 bp fragment was excised from pBAC5/H11 with SmaI/EcoRI and subcloned into the multiple cloning site of pBluescript-II-SK. This fragment contained the putative zBMP2 promoter from about 60 bp 5′ of the first splice site. A SacI-KpnI fragment was then excised from this plasmid and directionally cloned into pGEM-EGFP containing the modified GFP reporter gene (GM2, see Cormack et al., 1996) resulting in the construct pzBMP2(1.4)-EGFP as shown in FIG. 2.

We considered that the control of expression of zBMP2 gene likely resided in this SacI-KpnI fragment, and would be useful in controlling the “Sterile-Feral” construct. However, we are sure that any promoter with an appropriate spatial-temporal pattern could be used in the final “Sterile-Feral” construct. The construct pzBMP2(1.4)-EGFP was inserted into zebrafish embryos to test whether it followed a similar spatial-temporal expression pattern as reported for the zBMP2 promoter.

This construct and all subsequent constructs were prepared using the following procedures and introduced into the developing embryos by microinjection.

All the DNA preparations were appropriately linearized and gel purified (Qiaquick Gel Extraction Kit) before injection. Needles were made from borosilicate glass capillaries with filaments (GC100TF-15, Clark Electromedical instruments) using a P-80PC micropipette puller (Sutter Instrument Co.). The needle was back-filled with purified DNA diluted to 100 ng/μl in 1× injection buffer (10×; 50 mM Tris; 5 mM EDTA; 1M KCl, pH7.2) using a hand pulled pipette. Injections were carried out on a dissection microscope fitted with two, 3-dimensional Narshige MN-151 micromanipulators. Embryos were held in place during injection by a hydraulically (mineral oil) driven holding pipette. Injection of DNA solution was facilitated pneumatically using a 3-way foot operated plunge valve (Festo Engineering), connected between the injection needle holder and nitrogen tank. Injection was performed on one-cell stage embryos, unless specifically indicated otherwise. Injected embryos were incubated and reared as described above.

Post-injection, early-stage embryos were examined under UV illumination in a Zeiss microscope equipped with standard fluorescent isothiocynate (FITC) filter set, while later-stage embryos were anaesthetized in embryo medium containing 0.125%, 2-phenoxyethanol (Sigma P-1126), before examination. Photomicrographs of embryos expressing EGFP were obtained for analysis.

Table 1 summarises the injection trials. The percentage of embryos expressing EGFP at 10 h post injection (pi), varied from batch to batch, ranging from 0% to 42.7%.

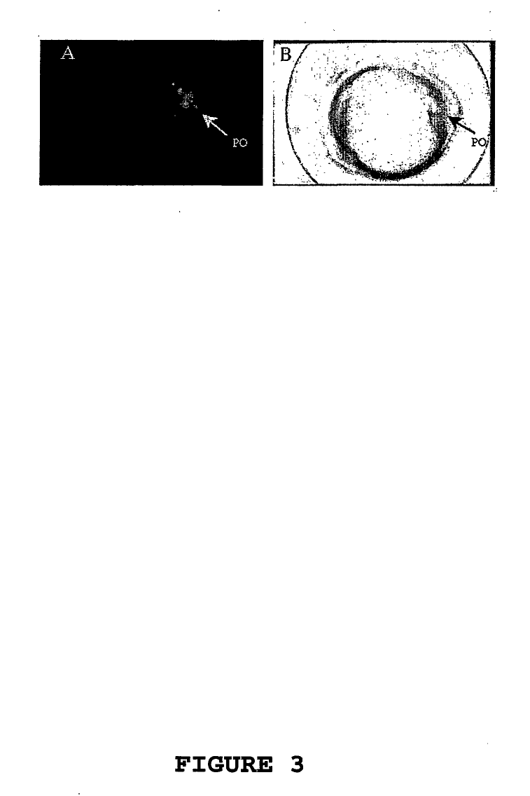

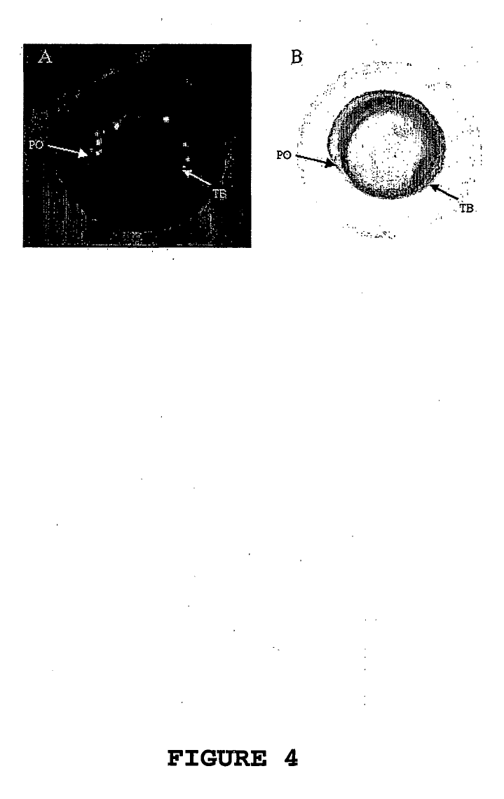

Expression was detectable as early as dorsal shield stage (6 h pi) in most of the expressing embryos. At 9.5 h pi, the majority of the expressing embryos had expression that was limited to anterior ventral regions (FIG. 3a); however, 3 embryos expressed EGFP all along the ventral margin (FIG. 4a). The patchiness is typical of the mosaic expression expected in founder transgenic animals. Nonetheless, expression domains extended from polster region (FIG. 3a;PO) anteriorly to the region of future tail bud, posteriorly (FIG. 3a;TB).

TABLE 1
Results of EGFP expression in embryos injected with
pzBMP2(1.4)-EGFP at about 9.5-10 h Post Injection

| | | | Number with | No. with |
|---|---|---|---|---|
| | | Total No. | Anterio- | entire |
| | Number | with | ventral | ventral |
| Batch | Observed | Expression | expression | domain |
| 1 | 28 | 0 | 0 | 0 |
| 2 | 21 | 3 | 1 | 2 |
| 3 | 20 | 2 | 0 | 2 |
| 4 | 28 | 12 | 2 | 10 |

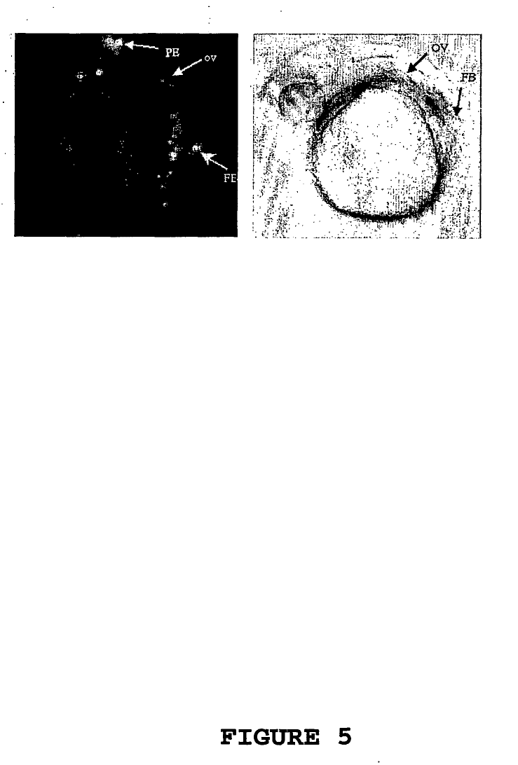

At about 24 h pi, expression was predominantly in the ventral domains (FIG. 5a), mimicking the native zBMP2 expression—in the region of the developing eye, otic vesicle, and pectoral fin bud. Abolition of tail bud expression at 24 h pi suggests that the cloned promoter may lack regulatory elements responsible for maintenance of BMP2 expression at this stage. No EGFP expression was detected by 48 h pi, suggesting that the zBMP2 gene is not required this late in development.

The zBMP2 promoter sequence is shown in SEQ ID NO:1.

### EXAMPLE 2

### Isolation of Second Promoter for Sterile Feral Construct

As the applicant was concerned about the potential shortcomings/delays of the BMP2 promoter in combination with a tet-responsive (tetOff) element to effectively block its own native transcripts, an early acting, but temporally restricted promoter sharing spatial domains with that of BMP2 was considered preferable. One such candidate was the zebrafish SMAD5. Similar to BMP2, mutation in the zebrafish SMAD5 results in a dorsalized mutation designated somitabun (sbn) and the dorsalised mutant phenotype has been shown to be rescued by injection of SMAD5 mRNA at the single cell stage (Hild et al., 1999). This indicated that the gene is essential only during early larval development. It has also been implied that the SMAD5 acts as a transducer of BMP2 signalling with potential upstream and downstream functions. The functional association between the BMP2 and SMAD5 suggested that the two genes share the same spatial expression domains. Further the maternal expression of SMAD5 and also the relative early onset of zygotic SMAD5 expression ensure that the cells are competent to process BMP2 signalling (Hild et al., 1999; Dick et al., 1999). Therefore, we considered that by employing a SMAD5 promoter to drive the expression of a BMP2 blocker would alleviate some of the potential temporal delays associated with employing the BMP2 promoter.

The cDNA for the SMAD5 gene was amplified from zebrafish shield stage cDNA using following primers

| | |
|---|---|
| SMADu1: | |
| 5′-TGCAGGTGGACTTTGGATCCG-3′ | SEQ. ID. NO.: 4 |
| SMADL1: | |
| 5′-GCCTAAAGGCAACAGATGCTA-3′ | SEQ. ID. NO.: 5 |

The primers were designed based on the published zebrafish SMAD5 cDNA sequences (Hild et al., 1999). The amplified 2285 bp product was cloned into pGem-T-Easy vector as per the cloning instructions of the manufacturer (Promega, Madison USA) and confirmed by sequencing. A resulting positive clone carrying the plasmid was grown according to standard protocols, and the cDNA from the bacterial culture was isolated by standard procedures. A 366 bp fragment spanning the 5′ untranslated region was isolated and labelled with ³²P. This was then used as a probe for a zebrafish genomic library.

Four positive clones (SMAD-BAC1, SMAD-BAC8, SMAD-BAC13, and SMAD-BAC 17) were then purchased from GSI. Preliminary sequencing of all four positive BAC clones using primers specific of the 5′-untranslated region of the cDNA revealed that the clones were identical to each other and to the region of the BMP2 cDNA. One of the BAC clones (SMAD-BAC51) was subcloned as HindIII fragments into pGEM-7ZF(+) by standard procedures. We obtained a positive subclone of about 8 KB (psBAC1/H12), that contained 1,005 bp of putative promoter sequence 5′ of the start codon. The coding sequence obtained was identical to the zebrafish SMAD5 cDNA sequence previously described by Hild et al. (1999).

A 1,005 bp putative promoter fragment was then amplified from psBAC1/H12 with the following primers

| | | |
|---|---|---|
| M13 forward: | 5′-GTAAAACGACGGCCAGT | SEQ ID NO: 6 |
| SMAD L2: | 5′-TAGTGCTGGGCTGCACCAG | SEQ ID NO: 7 |

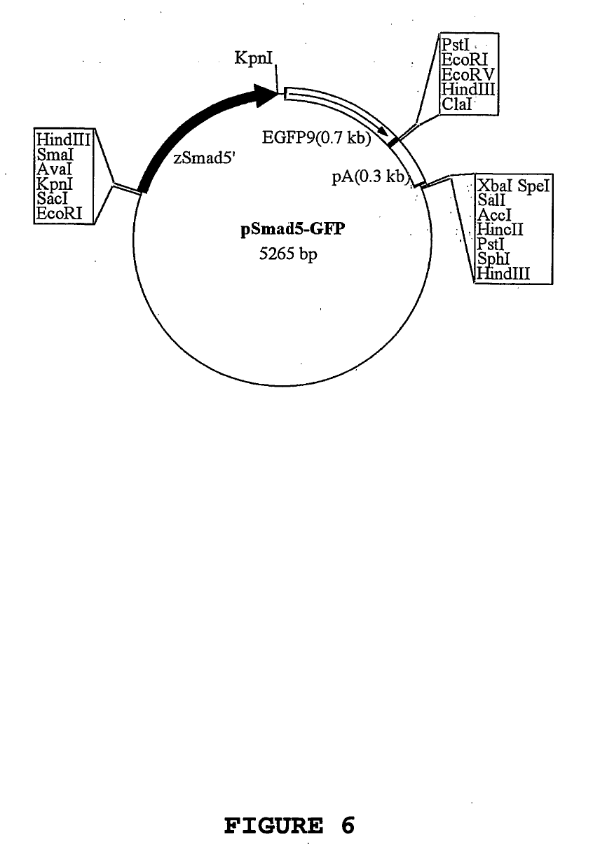

The amplified fragment was ligated into pGEM-Teasy vector and the orientation and sequence confirmed (pSMAD5′). The promoter was again excised as SmaI/EcoRI fragment, blunt ended and ligated into the SmaI linearized pGEM-EGFP. A positive clone, pSMAD5-EGFP (FIG. 6) in the correct orientation was selected and tested in vivo in zebrafish embryos.

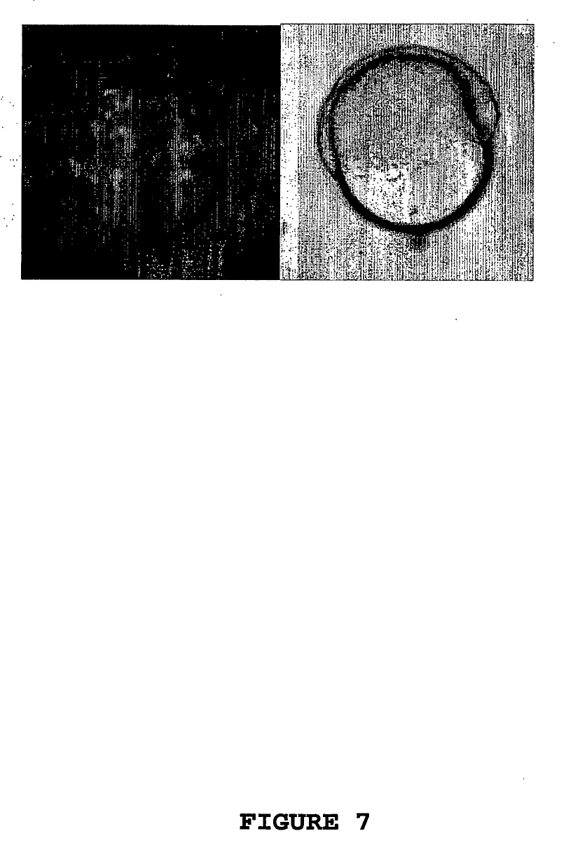

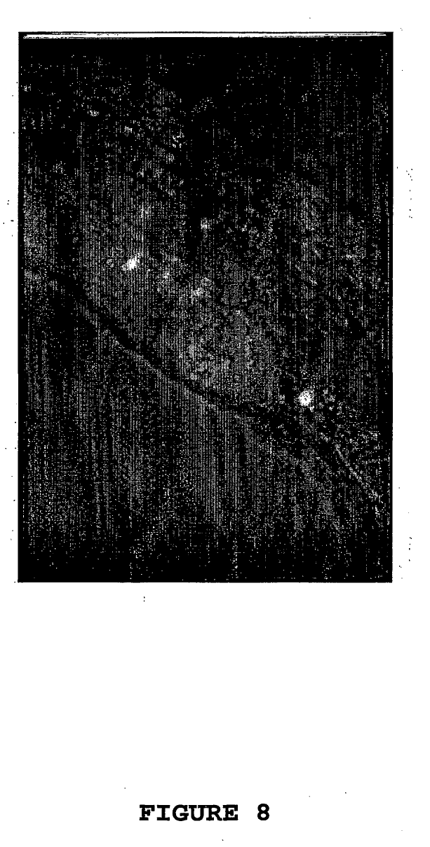

Injection trials of pSMAD5-EGFP into the zebrafish embryo resulted in expression of the EGFP as early as 4 hp. The expression pattern was ubiquitous initially as late as shield stage (FIG. 7), then predominantly restricting to ventral tissues at about 24 hpi (FIG. 8). The experimental evidence suggested that the zygotic expression of SMAD5 was activated marginally ahead of zBMP2. Although preliminary, our promoter analysis experiments suggested that the SMAD5 promoter was indeed activated slightly ahead of bmp2 promoter (data not shown). No EGFP expression was detected by 48 hpi, suggesting that the SMAD5 gene was not required this late in development.

The zebrafish SMAD5 promoter sequence is shown in SEQ ID NO; 8.

### EXAMPLE 3

### Zebrafish Model

Breeding and rearing protocols for zebrafish generally follow Westerfield (1995). Stock was obtained from a local pet store; however, it would be appreciated by those skilled in the art that zebrafish could equally be obtained from laboratories around the world (e.g., Institute of Neuroscience, eugene, Oreg., USA) and maintained at 27-28° C. in an in-house re-circulatory flow-through system. Embryos were obtained by natural matings, transferred into Embryo Medium (Westerfield, 1995), and incubated in a bench top incubator at 26-27° C. until 3-4 days old. They were then transferred into nursery tanks maintained at 27-28° C., and reared on finely ground commercial fish flakes (Tetramin), and live Artemia. After approximately 3 months, the fish were transferred into standard fish tanks alongside the adult fish. The adult fish were fed daily with flakes and occasionally supplemented with either freshly hatched or frozen Artemia.

### EXAMPLE 4

### Blocking Expression of zBMP2

The applicant tested three options for blocking expression of the candidate genes: mis/over-expression of sense (see below), antisense (Izant and Weintraub 1984) and double stranded RNA (dsRNA). (Guo and Kemphues, 1995). The latter appears to be more potent than antisense at inducing interference in C. elegans (Fire et al., 1998) and has been employed to silence native and reporter genes in plants (Waterhouse et al., 1998). To develop and optimise the blocking component of the “sterile feral” construct, the applicant assayed sense, antisense, and dsRNA of zBMP2 by injection in zebrafish embryos. Results indicated that both antisense and dsRNA block gene expression, whereas sense strand injection resulted in over-expression.

Capped full-length sense and antisense zBMP2 RNA transcripts were generated by linearizing the plasmid pzBMP2b, whereas the truncated versions of just the 5′- or the 3′-regions were generated by appropriately linearised pzBMP2-ApaI or pzBMP2-BstXI, respectively. All in vitro transcriptions were carried out using T3/T7 mMESSAGE mMACHINE™ (Ambion), as appropriate. dsRNA was prepared by annealing sense and antisense RNA in RNAase free injection buffer at 37° C. for 5 minutes for the truncated and 10 minutes for the full-length transcripts. Annealing of respective sense and antisense strands as dsRNA was confirmed by running a sample on a non-denaturing agarose gel. About 3-5 picolitres of RNA solutions, ranging between 100-250 ng/μl, were injected into 1-2 cell stage embryos as described above in Example 1. In the case of 2-cell stage injections, both the cells were injected.

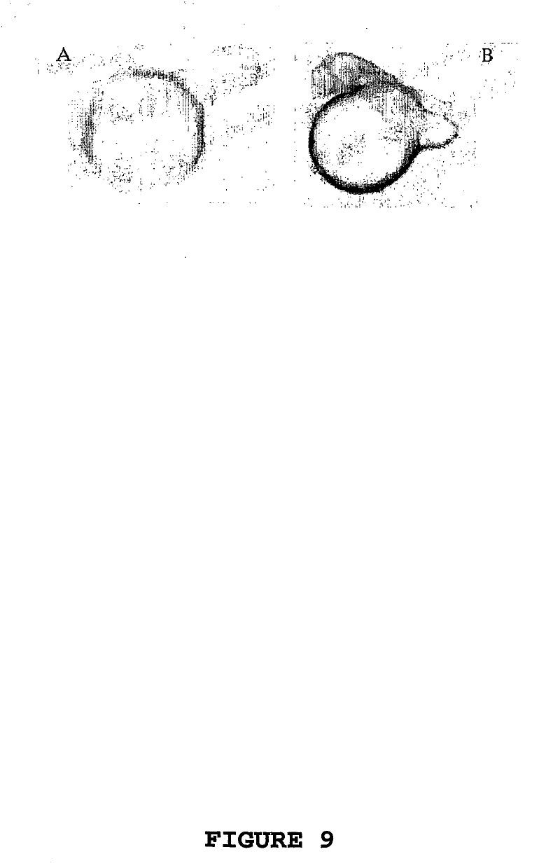

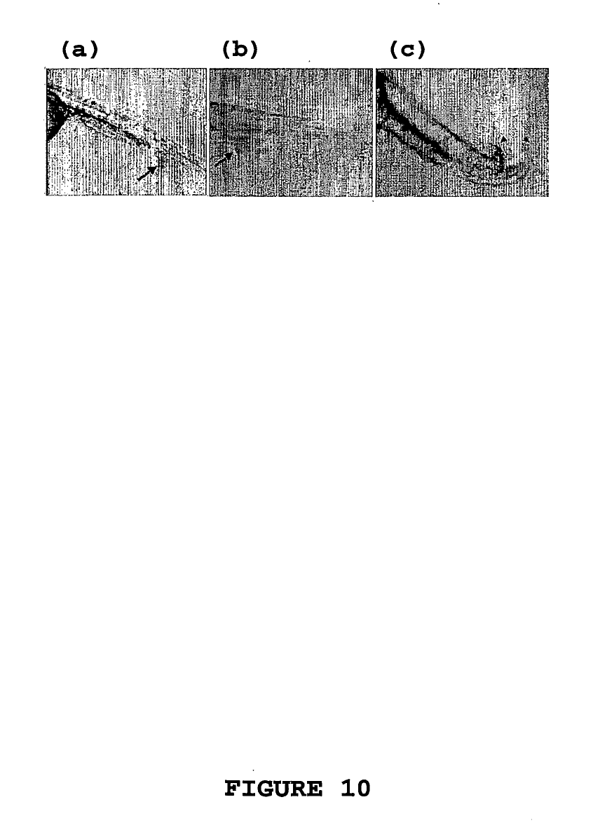

In embryos injected with full-length antisense or dsRNA of zBMP2, the proportion of normal embryos was significantly reduced and some weakly dorsalised embryos resembling zebrafish swirl mutant were seen (FIG. 9a&b). Sense injections resulted in mild ventralization of the embryos, which in some cases resembled the zebrafish chordino mutant phenotype (FIG. 10). Chordino is the dorsally expressing zebrafish homologue of chordin, known to interact antagonistically with BMPs (in this case swirl) in a dose dependent manner (Kishimoto et al., 1997).

To obtain molecular data to support hypothesised interference of the dsRNA on expression of zBMP2, the applicant injected truncated forms of zBMP2 ds RNA, so as to use the uninjected portion as probe to detect and quantify the native transcript levels in the injected embryos. The percentage of deformed embryos in groups injected with 3′-zBMP2 and 5′-zBMP2 dsRNA was 43.4% and 40.2%, as compared to 9.2% and 2.4% in the corresponding controls (Table 2).

TABLE 2
Results of Truncated zBMP2 dsRNA Injection
Into One-Cell Stage Embryos

| Transcript | | Number | Number | Number |
|---|---|---|---|---|
| Injected | Conc. ng/μl | injected | Survivors* | deformed* |
| 3′-zBMP2 | 150 | 123 | 83 | 36 |
| | | | (67.5) | (43.4) |
| Control | 0 | 66 | 54 | 5 |
| | | | (81.8) | (9.2) |
| 5′-zBMP | 250 | 88 | 67 | 27 |
| | | | (76.1) | (40.2) |
| Control | 0 | 53 | 42 | 1 |
| | | | (79.2) | (2.4) |

*Results in parenthesis indicate percentages

### EXAMPLE 5

### Combined Promoter and Blocker DNA Construct

On confirming the ability of in vitro transcribed BMP2 antisense and double stranded transcripts to disrupt larval development, DNA constructs capable of expressing the antisense and double stranded transcripts in vivo were developed and tested.

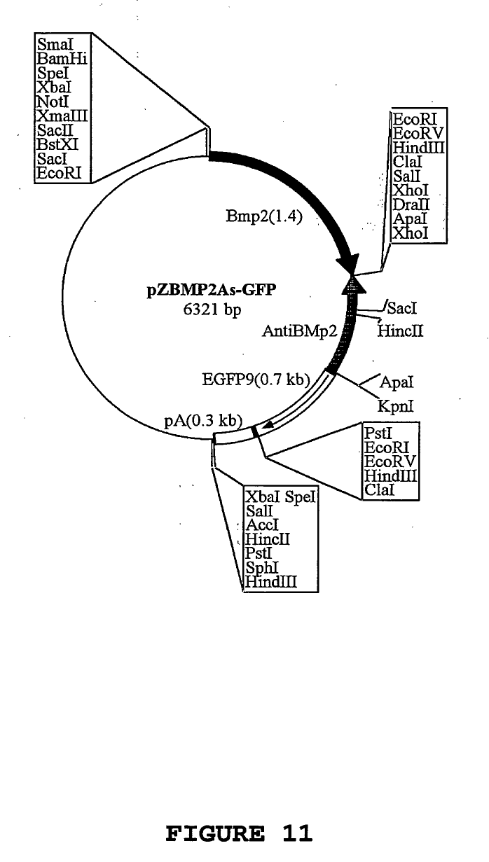

A 711 bp ApaI fragment of the zBMP2 cDNA was excised from the plasmid pzBMP2b and inserted into the ApaI linearized pzBMP2(1.4)-EGFP resulting in the pzBMP2As-EGFP (FIG. 11). Antisense orientation of zBMP2 fragment in pzBMP2AS-EGFP was confirmed both by restriction analysis and sequencing. The pzBMP2As-EGFP was a fusion construct capable of co-expressing BMP2 antisense and EGFP. Co-expression of EGFP with the BMP2 antisense provided an easily detectable marker to distinguish the mutant embryos emanating from antisense interference and those potentially resulting from spontaneous or background mutations. pzBMP2As-EGFP was linearized with NotI for injection into the embryos.

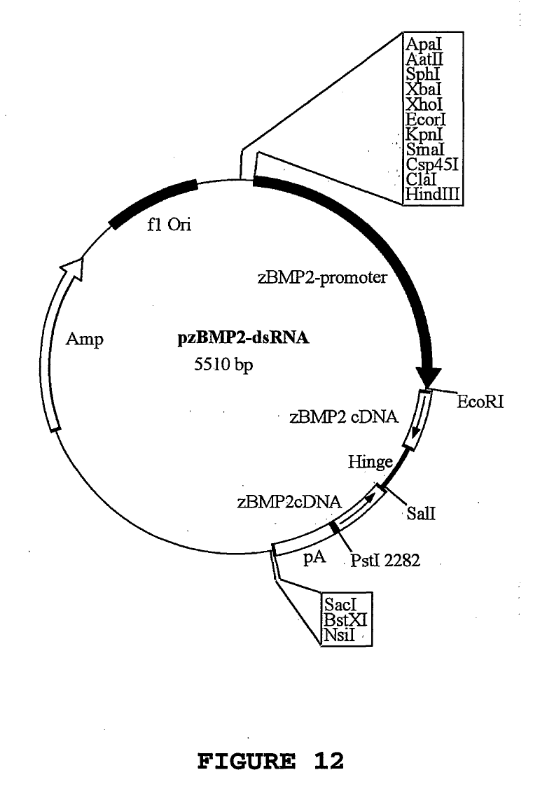

For the double stranded knockout, four segments of the zBMP2 gene were arranged to express double stranded mRNA in vivo (FIG. 12). The first-section comprised the 1,414 bp “HindIII-EcoRI” promoter region retained in the pGEM 7zf(+) vector backbone, obtained by excising the EcoRI-SacI coding region of the zBMP2 from pBAC5/H11 subclone. The second segment was a 510 bp fragment of the zBMP2 cDNA from sequence 301-810 in the published cDNA sequence (Lee et al., 1998). This fragment was amplified using the following primers:

| | |
|---|---|
| zfEx1-3.EcoF Forward Primer | |
| 5′-ACCCCGAATTCATGAGGAACTTAGGA-3′ | SEQ ID NO: 9 |
| zfEx1-3.SalR Reverse Primer | |
| 5′-ATCAGCTCGTCGACAGGAATGGAGGTAAG-3′ | SEQ ID NO: 10 |

The amplified product generated had an EcoRI site on the 5′-end and a SalI site on the 3′-end for ease of cloning. The third section was a 286 bp fragment of cDNA (bases 307-592) which was amplified using the following primers:

| | |
|---|---|
| Bex1i.PstF 2 Forward Primer | |
| 5′-ACACCTGCAGATGAGGAACTTAGGAGACGAC-3′ | SEQ ID NO: 11 |
| Bex1i.SalR Reverse Primer | |
| 5′-TACTGAGGGTCGACTGCCGATTTGCT-3′ | SEQ ID NO: 12 |

These primers generated a PstI site on the 5′ end and SalI site on the 3′ end for cloning. When ligated to the second fragment, the third segment formed an inverted repeat of the 5′ end of the cDNA (bases 307 through 592). The final segment was a PstI-SacI fragment containing a poly A tail section, excised from the pGT2-ns-GM2f construct that was kindly donated by Dr. Shou Lin, Institute of Molecular Medicine and Genetics, Medical College of Georgia. The DNA sequence for the double stranded BMP2 construct is given as SEQ ID NO:13.

Results of the BMP2 antisense-EGFP fusion construct injection are presented in the Table 3.

TABLE 3
Results of NotI linearized pzBMP2-As-EGFP Injection
into the One-Cell Zebrafish Embryos

| | | | | | Number |
|---|---|---|---|---|---|
| | Conc. | Number | Number | Number | with EGFP |
| Batch | μg/ml | injected | Survivors* | deformed* | expression |
| 1 | 100 | 48 | 36 | 1 | 0 |
| | | | (75) | (2.7) | |
| | 0 | 40 | 29 | 0 | 0 |
| | | | (72.5) | | |
| 2 | 100 | 36 | 16 | 6 | 5 |
| | | | (44.4) | (37.5) | (31.3) |
| | 0 | 16 | 9 | 0 | 0 |
| | | | (56.2) | | |
| 3 | 100 | 20 | 12 | 4 | 3 |
| | | | (60) | (33.3) | (75) |
| | 0 | 23 | 15 | 0 | 0 |
| | | | (65.2) | | |

*Figures in parenthesis indicate percentages.

The number of deformed individuals in the injected groups ranged from 0% to 37.5%. The majority of the deformed individuals (83.3% and 75% in batches 1 and 2, respectively) expressed EGFP, indicating that the antisense was effective in disrupting the larval development. None of the individuals in the control group and non-deformed individuals in the injected group had EGFP expression.

Results of the zBMP2-double stranded construct are given in Table 4.

TABLE 4
Results of pzBMP2-ds Injection into
1-4 Cell Stage Zebrafish Embryos

| | Treatment | Number | Number of | Number |
|---|---|---|---|---|
| Batch | Conc. (μg/ml | Treated | mortality | Deformed |
| Injected | 0 | 37 | 4 (10.8) | 0 |
| Control | | | | |
| Uninjected | — | 123 | 24 (20.5) | 1 (0.8)* |
| control | | | | |
| dsRNA injected | 100 | 143 | 20 (14.3) | 21 (14.7) |
| Uninjected | — | 51 | 11 (17.1) | 0 |
| control | | | | |
| dsRNA injected | 100 | 47 | 7 (16.5) | 22 (45.7) |

Figures in parenthesis indicate percentages.
*denotes a deformed control fish that had deformities that
did not resemble the swirl mutants.

Of 211 control embryos (mock-injected with buffer only or permitted to develop normally), only one embryo was deformed. The deformity did not resemble the swirl mutant. In the two dsDNA treatment groups, 14.7% and 45.7% of the embryos expressed the swirl mutation.

### EXAMPLE 6

### The Repressible Element

The proof-of-concept used a commercially available repressible element as the externally keyed genetic switch or Tet-responsive P_{hCMV*-1} promoter. P_{hCMV*-1} contains the Tet-responsive element (TRE) which consists of seven copies of the 42 bp tet operator sequence (tetO). This element is just upstream of the minimal CMV promoter (P_{minCMV}), which lacks the enhancer that is part of the complete CMV promoter. Therefore, P_{hCMV*-1} is silent in the absence of binding of transactivator protein (tTA) to the tetO. The tetracycline-sensitive element is described by Gossen and Bujard (1992; tet-off), Gossen et al. (1995; Tet-on), and Kistner et al. (1996). In the tetracycline-regulated system (Tet-Off system) developed by Hermann Bujard, addition of tetracycline (Tc) or doxycycline Dox; a Tc derivative) prevents the binding of a tTA, to the Tet-responsive element. This then blocks gene expression from the TRE until the drug is removed. A complementary system has also been developed (Tet-On system). In the Tet-On system, addition of doxycycline allows the binding of a reverse transactivater, rtTA, to the tetO promoter, leading to gene expression from the TRE. Gene expression continues from the TRE until removal of the drug. A tetracycline responsive element has the advantage of ease of administering. Tetracycline is a routinely used antibiotic in fish and shellfish culture (see Stoffregan et al., 1996), readily traverses cutaneous membranes while retaining its biological activity, and can be administered by whole organism immersion. Use of the Tet-On/Off controllable expression systems is covered by U.S. Pat. No. 5,464,758, assigned to BASF Aktiengesellschaft.

The applicant first tested the functionality of the Tet-off system in zebrafish cell cultures. The cell culture was established using ZF4 cells as previously described (Driever and Rangini, 1993). Cells were transfected with the DNAs using Effectene liposomes (Qiagen) according to the manufacturer's instructions. Cells were initially transfected with pTet-Off and placed under neomycin selection for 1 month. Neomycin-resistant cells were then transfected with pTRE-EGFP, and the selection plasmid pTK-Hyg, and placed under hygromycin selection for two weeks. EGFP expression was determined by examining and counting cells with obvious fluorescence and by examination of cell lysates using a fluorometer. Cells were grown in medium with or without doxycycline (0.2 μg/ml) for 72 h prior to assessment of gene expression, or were rinsed of doxycycline and assessed for reporter gene expression 72 h after removal of doxycycline.

In the absence of doxycycline, EGFP fluorescence was detected in a small percentage (approximately 6%) of cells (Table 5).

TABLE 5

| Transfection | % cells expressing | EGFP expression in |
|---|---|---|
| Treatment | EGFP | cell lysates |
| None | 0 | 0 ± 15 |
| pTet-Off | 0 | 0 ± 12 |
| pTRE-EGFP | 0 | 0 ± 9 |
| pTet-Off + pTRE- | 5.9 ± 1.2 | 86 ± 11 |
| EGFP | | |
| pTet-Off + pTRE- | 0.2 ± 0.1 | 5 ± 3 |
| EGFP + Dox (72 h) | | |
| pTet-Off + pTRE- | 2.6 ± 0.9 | 49 ± 6 |
| EGFP + removal of | | |
| Dox (72 h) | | |

Values represent the average and standard errors for 3 separate transfection experiments, each containing 4 replicates.

The low percentage of cells expressing the reporter gene presumably reflects the efficiency of simultaneously transfecting the cells with two plasmids (pTRE-EGFP and pTK-Hyg). When doxycycline was added, EGFP gene expression dropped substantially, to approximately 3% of expression levels seen in cells not exposed to doxycycline. Interestingly, washing the cells and removing as much of the doxycycline as possible could reverse the repression of reporter gene expression. Fluorometric assays of cell lysates performed using a BMG FluoStar showed similar results to cell counts, with repression of the EGFP fluorescence being repressed in the presence of doxycycline. The reversal of the repression following removal of doxycycline appeared greater in these assays, most likely because the fluorometer could detect low levels of fluorescence not detected by microscopic examination.

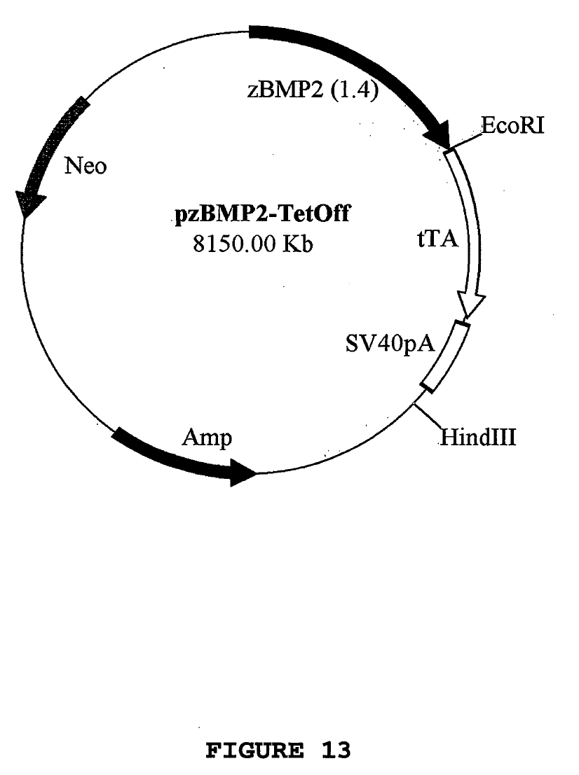

Next the applicant tested the tet-off system in whole zebrafish embryos. The Tet-On™ and Tet-off™ gene expression system and the Tet responsive bidirectional vectors pBI and pBI-EGFP were purchased from a commercial source (Clontech). The pzBMP2-Tet-Off construct (FIG. 13) was engineered by excising PminCMV promoter as SpeI and EcoRI fragment from pTet-Off and replacing it with the 1,414 bp zBMP2 promoter as XbaI/EcoRI, from pzBMP2-(1.4), by directional cloning. The pzBMP2-Tet-Off and pBI constructs were linearised with SacI and PuvII, respectively and column purified using a PCR purification column (Qiagen). Eluted DNA were quantified and mixed in equimolar ratio to yield a final concentration of about 150 ng/μl in injection buffer. Injections were carried out using one-cell stage embryos as described in Example 1.

Of the 84 embryos co-injected, EGFP expression was detectable in 7 (8.3%) individuals at about 24 h pi. A low percentage of transformed embryos is typical of co-injection experiments. The spatial pattern of EGFP expression (along the anterio-ventral regions) is similar to that we previously observed when EGFP was directly under the regulation of zBMP2 promoter.

### EXAMPLE 7

### Complete Zebrafish Sterile Feral Construct

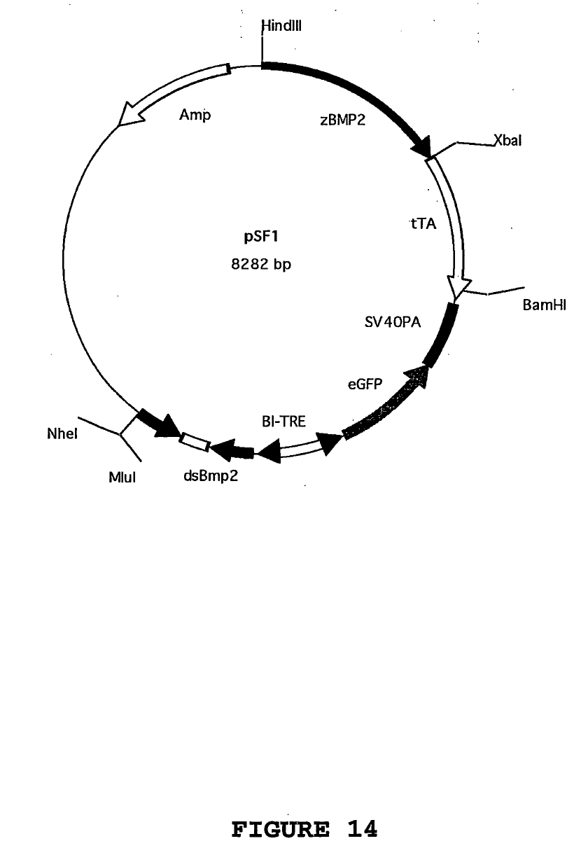

A single tet responsive double stranded RNA blocker construct under the regulation of zBMP2 promoter, pBIT(Bmp2)-Bmp2ds (FIG. 14), was built using pBI-EGFP as the backbone. The bidirectional tet responsive construct with EGFP as a marker was chosen to provide a visible marker. First, the SV40 PolyA was excised from the vector pBI-EGFP (Clontech, PT3146-5) following digestion with AatII and SalI. The resulting fragment was blunt ended with T4 DNA polymerase and religated to form pBi(−SV), an intermediate plasmid.

This was then cut with HindIII and used in a subsequent ligation with a HindIII fragment containing the BMP2 promoter, which was obtained from BMP-tetOff plasmid (SEQ ID NO:2, NM99/09099). The resulting plasmid, called pBi.tTA was then cut with with PvuII, dephosphorylated, and added to a ligation reaction containing a second fragment (blunt ended with T4 DNA polymerase), which contained the a 510 bp fragment of the zBMP2 cDNA from sequence 301-810 in the published cDNA sequence (Lee et al., 1998) and was obtained by digesting dsRNA (BMP2) (SEQ ID NO:13, NM99/09100) with EcoRI and HindIII followed by gel purification. This ligation reaction produced the construct pSF1. The pBIT(Bmp2)-bmp2ds construct is shown in FIG. 14 and SEQ ID NO: 14 and here through refereed to as pSF1.

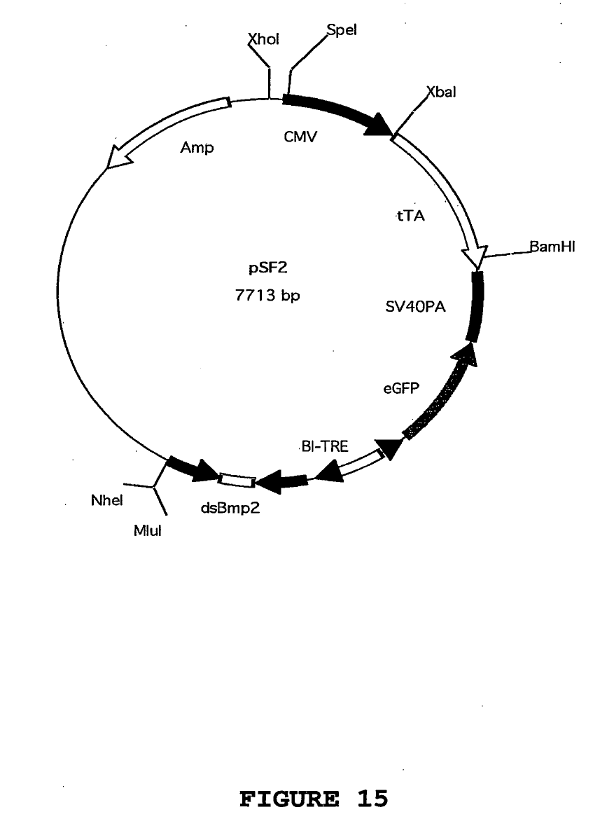

Similarly pBIT(Cmv)-BMP2ds (pSF2), a zbmp2 double stranded RNA blocker construct in which the tet-Off (tTA) is under the regulation of CMV promoter, was built as follows. Commercially purchased pTet-Off construct was digested with HindIII, XhoI and SapI. A 2250 bp XhoI/HindIII fragment containing CMV promoter, tTA and SV40 PolyA and a 2000 bp SapI/XhoI fragment containing vector backbone were gel purified. Meanwhile the pBIT(bmp)-bmp2ds was digested with HindIII/SapI and a 3,459 bp fragment containing EGFP and double stranded bmp2 RNA, with β-globin poly A was gel purified. Finally the three fragments were ligated directionally to yield the pBIT(CMV)-bmp2ds (pSF2, FIG. 15, SEQ ID NO:15) construct.

The applicant constructed two more candidate sterile feral constructs, with tTA driven by the zebrafish SMAD5 promoter: one used BMP2 double stranded RNA as developmental blocker [pBIT(smad)-BMP2ds] and another used zBMP2 sense, to be mis-expressed, as a blocker [pBIT(smad)-BMP2sense). An intermediate construct, pSmadTet-Off, was built by excising the CMVmin1 promoter as XbaI and SpeI fragmet from pTet-Off and replacing it with a 965 bp zebrafish SMAD5 promoter.

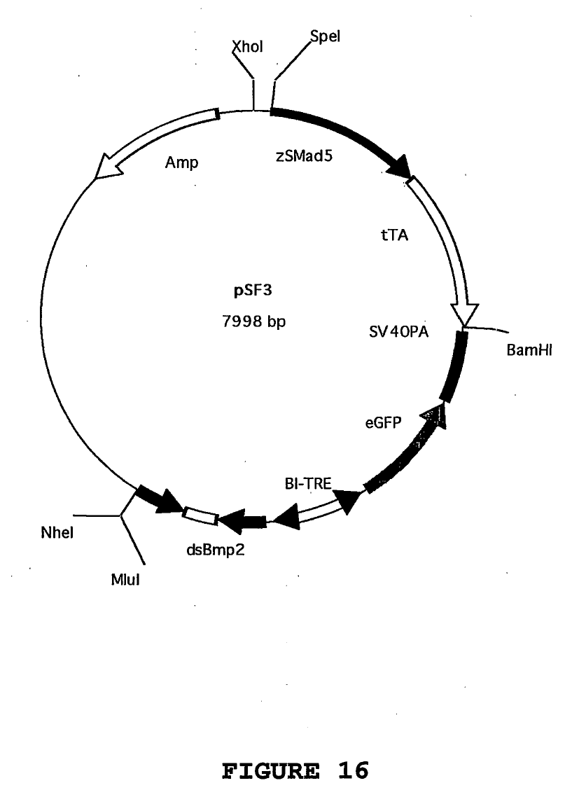

Subsequently, pBIT(smad)-BMP2ds (pSF3, FIG. 16, SEQ ID NO:16) was made by excising CMV promoter as a XhoI/SphI fragment from pBIT(CMV)-bmp2ds and replacing it with XhoI/SphI SMAD5 promoter fragment from pSmadTet-Off. The construct was confirmed by restriction analysis and sequencing. The construct was renamed pSF3.

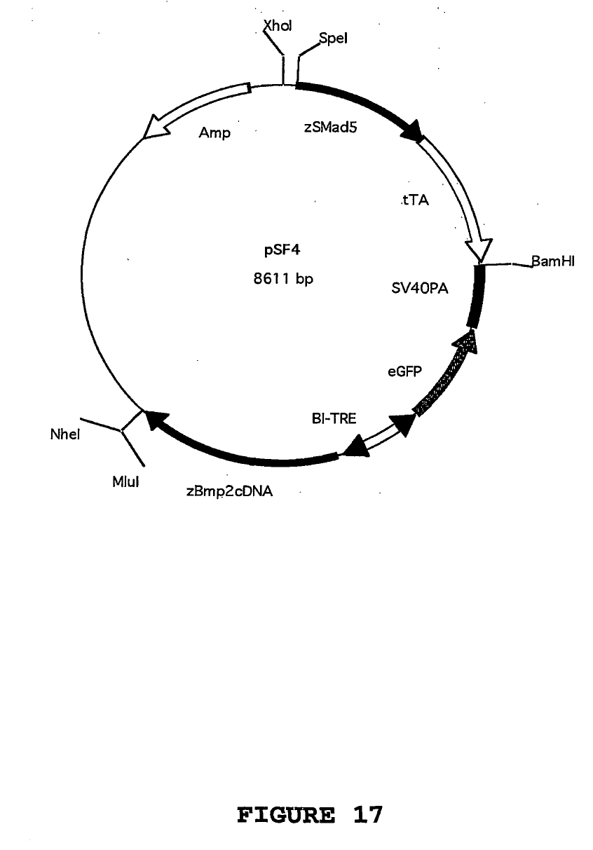

The pBIT(smad)-BMP2sense(pSF4, FIG. 17; SEQ ID NO:17) was constructed as follows. Firstly a 1,440 bp zebrafish BMP2 cDNA was excised as EcoRI and XhoI fragment from pzBMP2b, blunt ended and ligated into PvuII linearized pBI-EGFP. The sense orientation of the bmp2 cDNA in the bi-directional vector was confirmed by restriction analysis and sequencing. A resulting clone (pBI-bmp2-Sense) in the correct orientation was prepared for further use. The double stranded RNA blocker in the pBIT(smad)-bmp2ds (pSF3) was excised as EagI/MluI fragment and replaced with EagI/MluI fragment from pBI-bmp2-Sense construct. The resulting pBIT(smad)-bmp2-Sense construct (pSF4, FIG. 17 and SEQ ID NO:17) was confirmed by restriction analysis and sequencing.

Table 6 summarises the pooled results of three different batches of pSF1 construct injections into zebrafish embryos.

TABLE 6
Results of pSF1 (100 ng/μl) injections into the zebrafish embryo.

| | | No | No. | | | | | |
|---|---|---|---|---|---|---|---|---|
| | | dead | dead | No. | No. Glow | | No. Non Glow | |
| Treatment | Total | 5 hpi | 24 hpi | Live | Deformed | Normal | Deformed | Normal |
| SF1 | 166 | 65 | 11 | 90 | 2 | 34 | 0 | 52 |
| Injected | | | | (54.2) | (2.2) | (37.7) | | (57.7) |
| Buffer | 143 | 56 | 17 | 70 | 0 | 0 | 0 | 0 |
| Control | | | | (48.9) | | | | |

Although about 40% of the embryos had EGFP expression, only 2.2% had the associated deformity resembling the dorsalized swirl mutation. This is in stark contrast to 14-40% swirl like deformities the applicant observed by injection of a double stranded RNA construct (pzBMP2-ds) that was driven directly by the BMP2 promoter. The lack of correlation between the deformity and EGFP expression may be attributed to several reasons, including the delay associated with the indirect expression of the blocker by the BMP2 promoter mediated via the expression of tTA.

Table 7 summarises the results of pSF2 injected into the embryos of zebrafish.

TABLE 7
Results of injecting pSF2 (100 ng/μl) into the embryos of zebrafish.

| | | | No | No. | | | | | |
|---|---|---|---|---|---|---|---|---|---|
| | | | dead | dead | No. | No. Glow | | No. Non Glow | |
| Treatment | | Total | 5 hpi | 24 hpi | Live | Deformed | Normal | Deformed | Normal |
| SF2 | Dox | 175 | 44 | 30 | 101 | 3 | 8 | 6 | 84 |
| | | | | | (57.7) | (2.9) | (7.9) | (5.9) | (83.1) |
| | No Dox | 183 | 28 | 53 | 102 | 11 | 49 | 2 | 40 |
| | | | | | (55.7) | (10.7) | (48.0) | (1.9) | (39.2) |
| Control | | 118 | 23 | 14 | 81 | 0 | 0 | 0 | 0 |
| | Dox | | | | (68.6) | | | | |
| | No Dox | 107 | 13 | 18 | 76 | 0 | 0 | 0 | 0 |
| | | | | | (71.0) | | | | |

CMV, a ubiquitously active promoter, drives the pSF2. In all these sets of experiments, about half the injected and control fish were immersed in a solution of 150 ppm doxycycline (dox) to evaluate the efficiency of repression. The data were pooled from 3 separate sets of injections.

Following pSF2 injection and repression, the proportion of embryos expressing EGFP in the dox treated group was much lower from that of untreated group (11% vs 59%). These results confirm Example 6 that the applicant has achieved temporal control of genes under the regulation of tet responsive promoter in zebrafish.

However, as in case of pSF1, there was no correlation between the embryos expressing EGFP and those with a dorsalized deformity. Although the CMV is a ubiquitously expressing promoter, the applicant hypothesized that the mosaic distribution of injected construct may have precluded consistent expression in the BMP2 expression domains.

The results from injection and repression of pSF3, in which the tTA is driven by zebrafish SMAD5 promoter are presented in Table 8.

TABLE 8
Results of the repression experiment following injection of pSF3 and pSF2 constructs
(100 ng/μl) in the zebrafish embryos. Numbers in parenthesis are percentages.

| | | | Dead | Dead | | Glow | | | No Glow | | |
|---|---|---|---|---|---|---|---|---|---|---|---|
| Treatment | | Total | 5 HPI | 24 HPI | # Live | Deformed | Normal | Total | Deformed | Normal | Total |
| pSF3 | No Dox | 60 | 9 | 13 | 38 | 15 | 11 | 26 | 0 | 12 | 12 |
| | | | | | (63.3) | (39.4) | (28.9) | (68.4) | | (31.5) | (31.5) |
| | Dox | 52 | 11 | 14 | 27 | 5 | 5 | 10 | 1 | 16 | 17 |
| | 125 ppm | | | | (51.9) | (18.5) | (18.5) | (37.0) | (3) | (59.25) | (62.9) |
| Control | No Dox | 85 | 29 | 0 | 56 | — | — | — | 5 | 51 | 56 |
| | | | | | (65.8) | | | | (8.9) | (91.0) | (100) |
| | Dox | 86 | 24 | 20 | 42 | — | — | — | 0 | 42 | 42 |
| | 150 ppm | | | | (48.8) | | | | | (100) | (100) |
| pSF2 | No Dox | 58 | 11 | 6 | 41 | 8 | 16 | 24 | 2 | 15 | 17 |
| | | | | | (70.6) | (19.5) | (39.0) | (58.5) | (4.8) | (36.5) | (41.4) |
| | Dox | 58 | 18 | 14 | 26 | 1 | 2 | 3 | 9 | 14 | 23 |
| | 150 ppm | | | | (44.8) | (3.8) | (7.6) | (11.5) | (34.6) | (53.8) | (88.4) |

The applicant included pSF2 injections in this set of experiments as positive controls for repression. Repression of embryos injected with pSF3 were carried out in rearing medium containing 125 ppm dox, unlike the 150 ppm employed for pSF2 injected groups. This was because in preliminary experiments the applicant encountered higher mortality associated with 150 ppm dox and pSF3 injected embryos (data not shown).

As for pSF2, treatment with dox reduced substantially the percentage of surviving embryos exhibiting EGFP expression and swirl-like deformies, confirming repression. Unlike the pSF2 construct, there was a clear association between. EGFP expression and a dorsalizied mutation, the two co-expressing in close to 40% of the embryos surviving past 24 hpi. This confirms that the SMAD5 promoter effectively expressed the BMP2 double-stranded blocker, causing developmental arrest in un-repressed embryos. The applicant hypothesize that the increased efficiency of SMAD5 promoter in the complete Sterile Feral Construct over that of BMP2 promoter results from its potential early zygotic activation, ensuring the transcription of blocker molecules much before expression of the native BMP2 transcripts. Since the smad5 is known to be expressed maternally (Hild et al., 1999), it is likely to function even more effectively in permanently transformed lines.

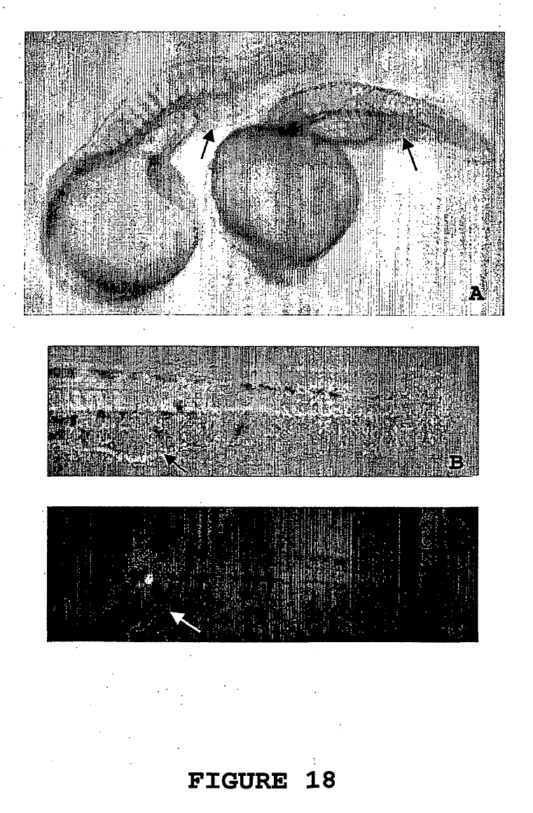

The applicant also built and tested a Sterile Feral Construct for zebrafish using mis-expression of the BMP2 gene as the blocker sequence (pSF4). As predicted, injection of pSF4 resulted in overexpression of BMP2, resulting in fish with ventralizied mutations (FIG. 18A-C, arrow. Majority of the deformed fish co-expressed EGFP and in some instances the EGFP expression was closely associated with the ventralized tissue (FIG. 18C). As summarized in Table 9, the large majority of the EGFP expressing embryos also had ventralized phenotypes as shown in FIG. 18A-C.

TABLE 9
Results of pSF4 injection (100 ng/μl) into zebrafish embryos

| | Total | No. Dead | | No | No Glowing | | No. non-Glowing | |
|---|---|---|---|---|---|---|---|---|
| Treatment | No. | 5 HPI | 24 HPI | Live | Deformed | Normal | Deformed | Normal |
| PSF4 | 234 | 104 | 37 | 93 | 33 | 31 | 7 | 22 |
| Injected | | (44.4) | (15.8) | (39.7) | (35.4) | (33.3) | (7.5) | (23.6) |
| Control | 118 | 46 | 10 | 68 | — | — | 3 | 65 |
| | | | | | | | (4.4) | (95.5) |

### EXAMPLE 8

### Transfection of Pacific Oysters

Mature oysters (Crassostrea gigas) were obtained from local hatcheries in Tasmania and New South Wales, and held in artificial seawater at 10° C. until required. Eggs were collected from 2-3 females by stripping the gonads and were pooled, rinsed on a 20 μm mesh, and left to condition in artificial sea water for 2 h. Sperm were stripped from male gonads, diluted to approximately 10,000 gametes/μl, and used immediately for electroporation-mediated nucleic acid delivery. Plasmid DNA (50 μg/ml) or double-stranded RNA (dsRNA; 1 μg/ml) was delivered into 1×10⁶ sperm using a BioRad Gene Pulser II electroporator in 0.2 cm gap electroporation cuvettes. Sperm were subjected to a single electroporation pulse (50 V, 100% modulation, 10 kHz, 12.5 msec) and immediately mixed with 5000 oocytes. Fertilized embryos and developing larvae were reared at 20° C. in artificial seawater containing 0.1 μg/ml chloramphenicol. Surviving larvae were counted after 24 h development. For experiments in which the Drosophila melanogaster heat shock promoter was used to drive expression of the delivered genes, a 1 h heat shock at 37° C. was provided either at 2 h or 18 h post fertilization, and development was then permitted to proceed at 20° C.

The applicant developed and tested transfection techniques for Pacific oyster eggs and larvae using genes encoding enhanced green fluorescent protein (EGFP, Clontech), glucuronidase (GUS), and red fluorescent protein (RFP, Clontech). Efficacy of electroporation as a transfection method of oyster sperm, using EGFP as a reporter gene was tested. Two different constructs, containing the EGFP gene under the control of either the CMV or Drosophila heat shock (Hsp) promoter were delivered into sperm using electroporation, and EGFP fluorescence was monitored using microscopy and fluorometric assays. Oyster embryos and larvae displayed a moderate level of autofluorescence that obscured detection of low levels of EGFP. Consequently, it was seldom possible to visually distinguish transfectants from non-transfectants when the EGFP gene was under the control of the CMV promoter using the construct pBiT(CMV)-EGFP (SEQ ID NO:18) as compared to EGFP expression levels observed using pBiT(dHSP)-EGFP (SEQ ID NO:19) following heat shock. However, EGFP and RFP were easily detected when expressed under the control of the D. melanogaster heat shock promoter, using constructs pBiT(dHSP)-EGFP (SEQ ID NO:19) and pBiT(dHSP)-RFP-oHoxDS/BH (SEQ ID NO:20) respectively. By visual inspection, it was estimated that approximately 60% of the surviving trochophore larvae were transfected (Table 10).

TABLE 10

| | Genetic | | | |
|---|---|---|---|---|
| Electro- | construct | | % larvae with | EGFP fluorescence |
| poration | Promoter/ | Heat | EGFP | relative |
| applied | Reporter | shock | fluorescence¹ | to controls |
| − | − | − | 0 ± 0.2 | 1 ± 0.2 |
| + | − | − | 0 ± 0.2 | 1 ± 0.2 |
| − | CMV/EGFP | − | 1 ± 0.5 | 1 ± 0.3 |
| + | CMV/EGFP | − | 5 ± 3 | 1.5 ± 0.2 |
| − | Hsp/EGFP | + | 4 ± 1 | 1.2 ± 0.3 |
| + | Hsp/EGFP | − | 24 ± 10 | 2.4 ± 0.7 |
| + | Hsp/EGFP | + | 61 ± 15 | 14.3 ± 1.1 |

¹Larvae with EGFP fluorescence visibly greater than that seen in non-transfected controls
The values represent the means and standard errors for three separate experiments.

To quantitatively assess EGFP and RFP fluorescence, larvae were homogenized in homogenization buffer (50 mM sodium phosphate, pH 7.0, 10 mM EDTA, 0.1% Triton X-100, 0.1% Sarkosyl, 10 mM mercaptoethanol), and protein extracts were measured for fluorescence using a BMG FLUOstar fluorometer.

Transfection efficiencies were also assessed in a second set of experiments that examined delivery of pHSP-GUS construct (SEQ ID NO:21). The pHSP-GUS construct was made in a two step fashion. First, the D. melanogaster heat shock promoter and terminator were isolated from the pCaSpeR-hs plasmid (Thummel and Pirrotta, 1992, Drosophila Information Service 71: 150) by PCR using the two primers:

| | |
|---|---|
| Dmhsp Forward Primer | |
| 5′-GAATTCCTAGAATCCCAAAACAAACTGG-3′ | SEQ ID NO: 31 |
| Dmhst Reverse Primer | |
| 5′- | |
| GGATCCTGACCGTCCATCGCAATAAAATGAGCC-3′ | SEQ ID NO: 32 |

The resulting amplicon was cloned into the T-tailed vector pGEM-T-Easy (Promega) according to the manufacturer's directions to produce the pGEMhsp70 plasmid. The second step involved excision of the gene encoding the β-glucuronidase gene (gus) from the plasmid pBacPak8-GUS (Clontech) using the restriction endonucleases NcoI and EcoRI. The ends of the 1.8 kb gus fragment were then converted to blunt ends using the Klenow fragment of E. coli DNA polymerase. The pGEMhsp70 plasmid was then linearized at the polylinker site between the promoter and terminator sequences using BglII and the ends were converted to blunt ends using the Klenow fragment. The 1.8 kb gus gene fragment was finally ligated into the blunt-end BglII site to produce the pHSP-GUS plasmid (SEQ ID NO:21). The pHSP-GUS construct expresses GUS under the control of the D. melanogaster heat shock promoter (Table 11).

TABLE 11
Efficacy of electroporation as a transfection method
of oyster sperm, using GUS as a reporter gene.

| | Genetic | | | | GUS |
|---|---|---|---|---|---|
| | construct | | | | activity |
| Electro- | Promoter/ | Heat | % | GUS | relative to |
| poration | reporter gene | shock | survival | activity¹ | controls |
| − | — | none | 100 ± 4 | 4.2 ± 0.3 | 1.0 |
| + | — | none | 95 ± 5 | 4.3 ± 0.3 | 1.0 |
| − | CMV/GUS | none | 93 ± 5 | 4.4 ± 0.4 | 1.0 |
| + | CMV/GUS | none | 92 ± 6 | 6.7 ± 0.8 | 1.6 |
| − | Hsp/GUS | yes | 92 ± 5 | 4.5 ± 0.5 | 1.1 |
| + | Hsp/GUS | none | 91 ± 6 | 10.5 ± 0.5 | 2.5 |
| + | Hsp/GUS | yes | 90 ± 4 | 83.2 ± 5.4 | 19.8 |

¹GUS activity expressed as fluorescence units/μg protein.
Values represent the mean and standard error for three separate spawning experiments, each with three replicates.

GUS activity in these experiments was measured using a fluorometric assay as previously described (Jefferson, R A 1987).

Fluorometric assays of larval extracts confirmed that electroporation of sperm could deliver foreign DNA into oyster embryos (Tables 10 and 11). In the absence of electroporation, little or no reporter gene expression was detected in transfected larvae. With electroporation, clear differences were observed in the relative strengths of the two different gene promoters tested. Expression of the reporter genes was approximately 1.6 times higher using the heat shock promoter, even in the absence of heat shock, compared to expression levels observed using the CMV promoter. With heat shock, reporter gene expression increased another 6-8 fold.

### EXAMPLE 9

### The Repressible Element in Oysters

Tet-Off™ control of EGFP expression was first assessed in oyster heart primary cell culture, using culturing methods previously described (Mol. Marine Biol. Biotech. 5: 167-174). Oyster cells were first transfected with the pTet-Off plasmid (Clontech, Genbank ACC# U89929)., using Effectene liposomes (Qiagen), and placed under neomycin selection for 2 weeks. The cells were then co-transfected with the pBI-EGFP reporter gene plasmid (Clontech #PT3146-5) and the selection plasmid pTK-Hyg (Clontech, GenBank Accession #: U40398). Dually transfected cells were then treated with 1 μg/ml doxycycline and EGFP expression was assessed 72 h later. Doxycycline was then removed from the medium, cells were washed in PBS, and incubated for a further 96 h to determine if EGFP expression had changed. It can be seen from Table 12 that a small percentage of cells were observed to express EGFP in the absence of doxycycline.

TABLE 12
Tet-Off ™ Control of EGFP Expression in Oyster Cell Culture

| Transfection and doxycycline (Dox) | % cells expressing |
|---|---|
| Treatment | EGFP |
| None | 0 |
| pTet-Off | 0 |
| pBI-EGFP | 0 |
| pTet-Off + pBI-EGFP (no Dox) | 2.2 ± 0.4 |
| pTet-Off + pBI-EGFP (+ Dox for 72 h) | 0 |
| pTet-Off + pBI-EGFP (+ Dox for 72 h, | 0.5 ± 0.2 |
| followed by removal of Dox for 96 h) | |

The low double transfection rates are presumably due to most cells acquiring the pTK-Hyg plasmid without acquiring the pBI-EGFP plasmid. Addition of doxycycline to the medium resulted in complete repression of the EGFP reporter gene expression. When the doxycycline was removed, the level of reporter gene expression increased after 96 h, indicating that the repression is reversible.

The results in Table 12 indicated that gene expression in oyster cells can be regulated using the Tet-Off™ system, and hence similar experiments were conducted in oyster larvae.

Oyster embryos were transfected with the pBiT(HSP)-EGFP plasmid (SEQ ID NO:19), which encodes the tetracycline (or doxycycline)-controlled transactivator (tTA=Tet-Off™) under control of a heat shock promoter, and contains the EGFP reporter gene under the control of the tetracycline (doxycycline) response element (TRE). The construct pBiT(HSP)-EGFP (SEQ ID NO:19) was prepared as follows. Four fragments were prepared and ligated together to create the construct. The first, was obtained by digesting pHSP70-1MCS (SEQ ID NO:22) with XhoI and XbaI followed excision and gel purification of the appropriate XhoI/XbaI fragment containing the Drosophila HSP70 promoter. The second was obtained by digesting pTet-Off (Genbank ACC# U89929) with XbaI and HindIII and gel purifying the appropriate fragment containing the tet-responsive transcriptional activator (tTA) and SV40 poly adenylation signal. The third fragment was obtained by digesting pBI-EGFP (Clontech, PT3146-5) with HindIII and SapI and gel purifying the appropriate fragment containing the TRE and CMVmin bidrectional promoter and multiple cloning site. The fourth fragment was obtained by digesting pTet-Off (Genbank ACC# U89929) with XhoI and SapI and gel purifying the appropriate fragment containing the vector backbone and ampicilin resistance gene.

The construct expresses the tet-responsive transcriptional activator (tTA) from the Drosophila HSP70 promoter (PHSP70) which in turn activates expression of EGFP under control of the tetracycline-response element, or TRE. Oyster sperm were transfected with the construct using electroporation, and oocytes were fertilized and allowed to develop for 24 hours in the presence or absence of 5 μg/μl doxycycline. In the absence of doxycycline, EGFP was expressed in transfected oyster larvae, and when doxycycline was added, the EGFP expression levels dropped to levels equal to that of non-transfected embryos (Table 13). The results from the tissue culture and embryo transfections indicate that transgene expression in oysters can be effectively controlled using the Tet-Off™ system.

TABLE 13
Regulation of EGFP Expression Using Doxycycline in Oyster
Larvae Transfected with pBiT(HSP)-EGFP (SEQ ID NO.19)

Fluorescence (FU/μg protein)

| | Total fluorescence | |
|---|---|---|
| | (incl. | Corrected for |
| Treatment Regime | autofluorescence) | autofluorescence |
| Non-transformed | 320 (±21) | 0 (±21) |
| control | | |
| −Dox, − heat shock | 426 (±24) | 106 (±24) |
| −Dox, + heat shock | 1025 (±78) | 705 (±78) |
| +Dox, + heat shock | 215 (±27) | 0 (±27) |

Values represent the mean and standard deviation for two separate spawning experiments, each with three replicates.

### EXAMPLE 10

### Blocking Expression of a Developmental Gene in Oysters

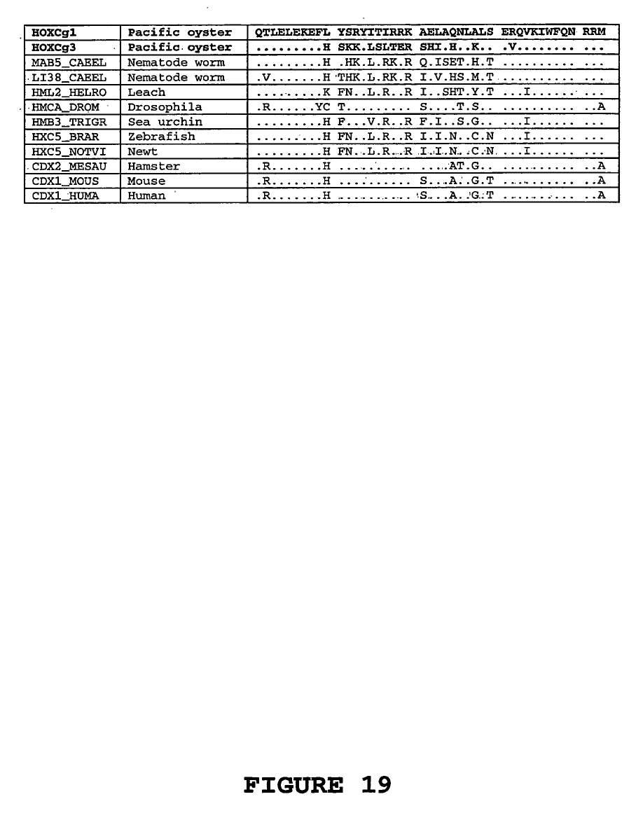

The applicant has identified conserved gene functions which are crucial to larval development in oysters and characterised two suitable candidate sequences as targets for antisense or dsRNA knockout. Disrupting this gene function is then lethal to the animal (larvae) because transcription factors are prevented from binding and initiating cascades of gene activity required for morphogenesis (body construction). The applicant chose to target the DNA binding ability of a class of transcription factors known as “Helix-loop-Helix” factors that bind DNA during the development of animal body plans (reviewed by Stein et al., 1996; and also see de Rosa, 1999). The applicant isolated two partial gene sequences comprising this crucial and highly conserved DNA binding sequence from a Pacific oyster cDNA library (HoxCg1 and HoxCg3; SEQ ID NOS.: 23 and 24, respectively). Alignments of the sequence of this evolutionary conserved class of genes and phylogenetic analysis have revealed that this sequence is indeed a HOX gene and is previously undescribed in oysters (FIG. 19).

The applicant identified two oligonucleotide sequences that are candidates for antisense larval pesticides. An oyster specific antisense:
5′-GAGATCGTTCAGTCAGCG-3′ SEQ ID NO:25.
   and a broader spectrum antisense
5′-CATGSGSSGGTTTTGGA 3′ SEQ ID NO:26.
   wherein “S” represents the base guanine or cytosine. These sequences are potentially capable of truncating vital gene products, and hence preventing their function in vivo.

The applicant synthesized and tested antisense and double stranded blockers for the gus gene from Escherichia coli, Hox CG1 (SEQ ID NO:23), and Hox CG3 (SEQ ID NO:24). RNA was prepared by in vitro synthesis for these three different genes or gene fragments: the 1.8 kb open reading frame of the gus gene from E. coli; the 129 bp fragment of oyster gene Hox Cg1 (SEQ ID NO:23, AGAL ref# NM99/09101); and the 129 bp fragment of the oyster gene HoxCG3 (SEQ ID NO:24, AGAL Ref#NM99/09102). The DNA fragments were each cloned into pBluescript SK(+), the vectors were linearized with either HindIII or PstI, and T3 or T7 RNA polymerase (Promega) was used to generate sense or antisense RNAs, respectively using a commercially available in vitro transcription kit (Promega, Madison Wis.). The resulting samples were then digested with DNase I for 15 minutes at 37° C. To produce double stranded RNA (dsRNA), equimolar amounts of the sense and antisense RNAs were mixed and heated to 80° C. and allowed to cool slowly to room temperature thus forming dsRNA. The RNA was extracted with phenol/chloroform and then chloroform, precipitated with ethanol, and resuspended in 10 mM Tris-HCl, pH 9. Formation of dsRNA was confirmed by resolving the annealed and non-annealed RNAs on a 1% agarose gel in TBE (90 mM Tris-borate, 2 mM EDTA, pH 8.0).

The in vitro transcribed dsRNAs, plus sense, and antisense RNAs for the GUS, HoxCG1 and HoxCG3 genes were delivered into oyster sperm by electroporation using a set of conditions previously found to be optimal for delivery of a reporter gene construct (dHSP70-GUS). Transfections for the control treatments were carried out in RNA free sea water. Delivery of sense and antisense RNAs had no or only a small effect on the number of individuals that developed, relative to the non-treated controls (Table 14).

TABLE 14
Effect on Early Larval Development of Oyster Transfected
with In Vitro Transcribed Sense (S), antisense (AS),
and double-stranded (DS) RNAs of three different
gene sequences, GUS, HoxCG1, HoxCG3

| RNA delivered | % survivors at 24 h | % arrested |
|---|---|---|
| into sperm | development¹ | development² |
| control | 100 ± 3 | 5 ± 1 |
| GUS - (DS) | 94 ± 5 | 7 ± 3 |
| HoxCG1 - (S) | 91 ± 5 | 9 ± 4 |
| HoxCG1 - (AS) | 85 ± 9 | 17 ± 5 |
| HoxCG1 - (DS) | 71 ± 7 | 79 ± 10 |
| HoxCG3 - (S) | 92 ± 4 | 8 ± 4 |
| HoxCG3 - (AS) | 87 ± 6 | 15 ± 3 |
| HoxCG3 - (DS) | 79 ± 7 | 23 ± 5 |

¹Percentage of embryos that developed into trochophores,
relative to non-treated controls
²Includes all individuals (embryos and larvae) that
failed to develop to the D-hinge larval stage

Transfection with dsRNA for the GUS gene had no obvious effect on development, but transfection with dsRNAs specific to the HoxCG genes resulted in increased numbers of individuals showing arrested early larval development. The dsRNA specific to the HoxCG1 gene was the most effective dsRNA, with almost 80% of individuals failing to develop beyond the trochophore stage of larval development (Table 14).

Screening for mutant phenotypes in the resulting larvae revealed severe developmental mutants especially in the treatments containing dsRNA for both gene constructs, but not the RNA-free controls (FIG. 20, Table 14). Fatal embryonic distortions due to the double stranded blocker of HoxCG1 can be broadly classified as defects in the anterior/posterior axis formation including associated structures (such as the velum) and for HoxCG3 as defects in velum and body—perhaps premature velum release.

To test whether dsRNA could reduce expression of a gene in oyster cells, primary cell cultures were first transfected with the pHSP-GUS plasmid (SEQ ID NO:21). After two days of growth, the dsRNA specific to the gus gene was delivered into these cells by transfection using Effectene liposomes (Qiagen). After 72 h, the level of GUS activity was measured. The cells transfected with the dsRNA showed a 76% reduction in the reporter gene activity compared to similarly aged gus-transfected cells (Table 15).

TABLE 15
Reduced GUS Transgene Expression in Oyster Cells
Transfected with In Vitro Transcribed dsRNA

| GUS Gene Expression | | |
|---|---|---|
| (ρmol MU produced/min) | | % decrease in |
| No dsRNA added | dsRNA added | gene expression |
| 42 ± 13 | 10 ± 4 | 76 |

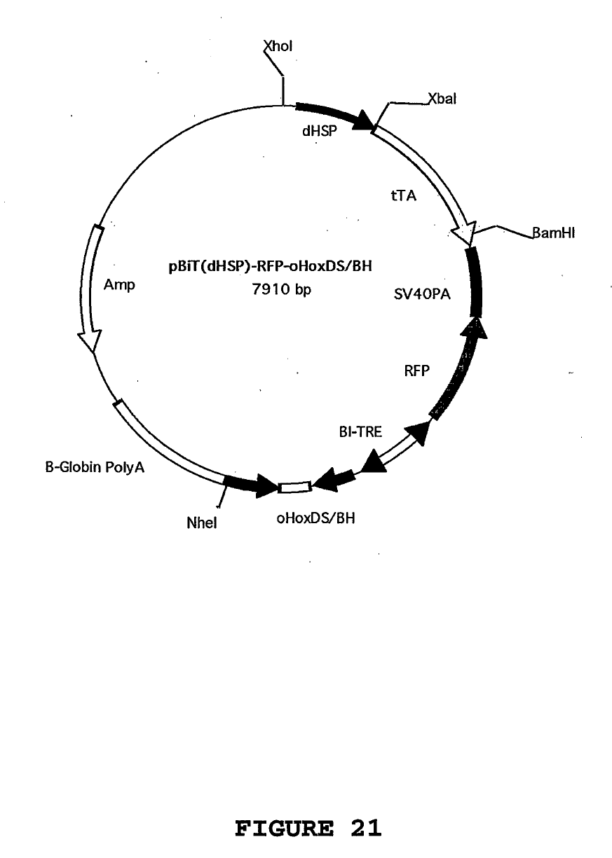

In vivo expression of dsRNA was achieved by transfecting oyster larvae with the pBiT(dHSP)-RFP-oHoxDS/BH plasmid (FIG. 21; SEQ ID NO:20), which contains the heat inducible promoter (P_{HSP70}) from D. melanogaster driving the expression of a hairpin RNA molecule specific to the HoxCG1 gene. The construct was prepared as follows. SEQ ID NO:23 (AGAL ref #: NM99/09101) was used as a template to generate a PCR fragment using the following primers:

| | |
|---|---|
| CG1.1.Sal.for Forward primer: | |
| 5′-ATGGATGTCGACTCAGACGCTGGAG-3′ | SEQ ID. NO.: 27 |
| And | |
| CG1.1.Pst.rev Reverse primer: | |
| 5′-GATTCACTAGTCAATTCCTGCAGTT-3′ | SEQ ID NO: 28 |

This fragment was then cloned into the pCR®2.1-TOPO (Invitrogen) cloning vector. Two separate fragments, an EcoRI/EcoRI and a SalI/PstI, both containing the HoxCG1.1 (SEQ ID NO:23), were digested out of this construct for use in further ligations. The latter fragment (SalI/PstI) was inserted into the dsRNA(BMP2) construct (AGAL ref# NM99/09100) which had been digested with SalI and PstI to remove the inverted BMP2 sequence. This intermediate construct was then digested with EcoRI and SpeI to produce a fragment containing both the a 510 bp fragment of the zBMP2 cDNA from sequence 301-810 in the published cDNA sequence (Lee et al., 1998) and the Hox CG1.1 (SEQ ID NO:23) fragment. This EcoRI/SpeI fragment and the EcoRI/EcoRI fragment containing HoxCG1.1 were then combined into a ligation reaction with pHSP70-1MCS (SEQ ID NO:22, containing the Drosophila heat shock promoter dHSP70 and its poly adenylation signal) digested with EcoRI and XbaI, to produce pHSP-oHoxDS/BH (SEQ ID NO:29). This latter construct uses the Drosophila heat shock promoter to drive expression of an mRNA consisting of an inverted section of the HoxCG1.1 followed by a section of BMP2 cDNA in sense orientation followed by a segment of the HoxCG1.1 fragment in sense orientation followed by the poly adenalation signal of the Drosophila heat shock promoter.

Oyster sperm were transfected with the DNA using electroporation, and oocytes were fertilized and larvae allowed to develop for 96 hours. Embryos were heat shocked for one hour at 3 hours post fertilization to induce transcription of the dsRNAs. Even without heat shock, approximately a third of the larvae failed to develop beyond the trochophore larval stage, and died within a few days (Table 16).

TABLE 16
Arrested Development of Oyster Embryos Transfected
with pHSP-oHoxDS/BH plasmid (SEQ ID NO: 29)

% arrested development

| | no heat shock | with heat shock |
|---|---|---|
| non-transfected | 5 ± 1 | 4 ± 1 |
| phsp-GUS | 6 ± 2 | 8 ± 3 |
| pHSP-oHoxDS/BH | 33 ± 9 | 67 ± 16 |

With heat shock, over 65% of the larvae failed to develop. Since all larvae are not transfected by the electroporation procedure, it is likely that those individuals that developed normally were not transfected with the genetic construct. Non-transfected oyster embryos and embryos transfected with a plasmid expressing dsRNA for the GUS gene showed no obvious reduction in survivorship (Table 16).

### EXAMPLE 10

### Complete Sterile Feral Construct for Oysters

Two different plasmids were prepared that used Tet-Off™ to control the in vivo expression of dsRNAs specific to developmental genes. The first, pBiT(CMV)-EGFP-zfBMP(DS), (SEQ ID NO:30), was designed to express the reporter gene EGFP as well as dsRNA specific to the zebrafish BMP2 gene in the absence of tetracycline or doxycycline. The construct was prepared as follows:

An intermediate constuct was first engineered using three separate fragments. The first was an XhoI/HindIII fragment that was obtained by digesting pTet-Off (Genbank ACC# U89929) with XhoI and HindIII and gel purifying the appropriate fragment containing the CMV promoter, tet-responsive transcriptional activator (tTA), and SV40 poly adenylation signal. The second fragment was obtained by digesting pBI-EGFP (CLONTECH) with HindIII and SapI and gel purifying the appropriate fragment containing the TRE and CMVmin bidrectional promoter and multiple cloning site (MCS). The third fragment was obtained by digesting pTet-Off (Genbank ACC# U89929) with XhoI and SapI and gel purifying the appropriate fragment containing the vector backbone and ampicilin resistance gene. These three fragments were ligated together to form the intermediate construct pBiT(CMV)-EGFP (SEQ ID NO:18). A fourth fragment, obtained by digesting Seq.ID#4 (dsRNA(BMP2), AGAL Ref# NM99/09100) with EcoRI and HindIII and gel purifying the appropriate fragment containing a 510 bp segment of the zBMP2 cDNA from sequence 301-810 and the inverted 286 bp segment of the cDNA (Bases 307-592) of the published zebrafish BMP2 cDNA sequence (Lee et al., 1998). This EcoRI/HindIII fragment was then blunt ended with T4 DNA polymerase and ligated into the unique PvuII site of the MCS of pBiT(CMV)-EGFP to form the construct pBiT(CMV)-EGFP-zfBMP(DS) (SEQ ID NO:30). This construct expresses the tet-responsive transcriptional activator (tTA) from the strong immediate early promoter of cytomegalovirus (P_{CMV}). The tTA functions to drive gene expression via the tetracycline-response element, or TRE. In the absence of tetracyline or doxycyline both EGFP and the blocker gene (double stranded BMP2 mRNA, cloned into the MCS) are expressed.

Sperm were transfected with either pBiT(dHSP)-EGFP (SEQ ID NO:19) or pBiT(CMV)-EGFP-zfBMP(DS) DNA, (SEQ ID NO:30), oocytes were fertilized, and allowed to develop for 24 hours in the presence or absence of 5 μg/μl doxycycline. Embryos transfected with the pBiT(dHSP)-EGFP DNA were not heat shocked so that EGFP expression would be similar in both transfections. When oyster embryos were transfected with this construct, lower hatch rates and poorer larval survival rates than those of non-transfected controls were observed (Table 17).

TABLE 17
Tet-Off ™ Control of EGFP and dsRNA-zfBMP Expression in
Oyster Embryos

| | % survival | | | |
|---|---|---|---|---|
| | (relative to | | EGFP (FU/μg | |
| Construct | control) | | protein) | |
| injected | −Dox | +Dox | −Dox | +Dox |
| Non-transfected | 100 + 5 | 100 + 3 | 0 + 10 | 0 + 11 |
| pBiT(dHSP)-EGFP | 77 + 6 | 95 + 3 | 31 + 8 | 0 + 8 |
| pBiT(CMV)-EGFP- | 71 + 8 | 92 + 4 | 20 + 11 | 0 + 9 |
| zfBMP(DS) | | | | |

When doxycycline was added to the water, this trend was reversed. Most of this arrested development however, may be caused by expression of EGFP, as similar levels of arrested development were observed when embryos were transfected with the pBiT(dHSP)-EGFP plasmid (without exposure to heat shock), and normal developmental rates were restored when doxycycline was added to the water. It cannot be excluded however, that the zebrafish dsRNA has caused some small degree of developmental arrest in the oysters, as the BMP2 may have an as yet unidentified orthologue with enough sequence identity to zfBMP2 to be affected by this dsRNA molecule.

The second Sterile Feral Construct tested for oysters, expresses the tTA under the Drosophila HSP. The tTA then drives expression of red fluorescent protein and double stranded oyster Hox via the TRE. Three separate fragments were ligated together to form this construct. The first fragment was obtained by digestion of pBiT(dHSP)-EGFP, (Seq ID NO:19), with HindIII and NheI followed by gel purification of the appropriate fragment containing the Drosophila HSP promoter. The second fragment was obtained by digesting pBiT(dHSP)-EGFP with NotI and MluI followed by gel purification of the appropriate fragment containing the TRE. The third fragment was obtained by digesting pHSP-oHoxDS/BH with MluI and. SpeI and gel purifying the appropriate fragment containing the 510 bp fragment of the zBMP2 cDNA from sequence 301-810 in the published cDNA sequence (Lee et al., 1998). The fourth fragment was obtained by firstly subcloning into pGEM3zf a KpnI/XbaI fragment containing the coding region of red fluorescent protein (RFP) that was excised from pDsRed1-N1 (Clontech, PT3405-5) vector. The resulting plasmid was then subjected to digestion with HindIII and PspOMI and the appropriate fragment containing the coding region of RFP was then gel purified from this reaction. This HindIII/PspOMI fragment was combined with the NheI/HindIII, NotI/MluI, and MluI/SpeI fragments to form the second sterile feral oyster construct pBiT(dHSP)-RFP-oHoxDS/BH (SEQ ID NO:20; FIG. 21).

Sperm were transfected with the plasmid, oocytes were fertilized, and allowed to develop for 72 hours in the presence or absence of 5 μg/μl doxycycline. When oyster embryos were transfected with the second repressible sterile feral construct, a considerable percentage (67%) failed to develop beyond the trochophore stage of larval development and subsequently died before reaching the D-hinge stage (Table 18).

TABLE 18
Reversible Arrested Oyster Larval Development Following
Transfection with the Tetracycline-Responsive Plasmid
phsp-BiT-RFP/dsRNA-HoxCG1

| | |
|---|---|
| Construct used for | % arrested development |

| transfection | No doxycycline | With doxycycline |
|---|---|---|
| Non transfected | 0 ± 5 | 0 ± 3 |
| phsp-GUS | 5 ± 3 | 4 ± 3 |
| pCMV-RFP | 5 ± 2 | 4 ± 3 |
| phsp-BiT-dsRNA- | 67 ± 8 | 9 ± 4 |
| HoxCG1/RFP | | |

Addition of doxycycline to the water virtually prevented the developmental arrest, and most embryos developed properly to the D-hinge larval stage, relative to the non-treated controls.

RFP expression was not easily detected by microscopy in embryos transfected with the RFP gene under the control of either a heat shock or a CMV promoter. A small amount of RFP was detected using fluorometric measurements of larvae transfected with the pCMV-RFP construct, but little RFP could be detected in larvae transfected with the repressible anti-development construct (results not shown). As many of the embryos transfected with this latter construct fail to develop, the lack of RFP expression is not surprising. Attempts to detect RFP in early and late staged embryos were unsuccessful, using either RFP-expressing construct.

### EXAMPLE 11

### Development of a Repressibly Sterile Mouse

Development of the sterile feral construct for mice parallels that detailed above for zebrafish, and involves identification of a suitable target gene and associated promoter, engineering these into a construct with the Tet On/Off repressible system, and then-testing, in this case in cell lines, prior to production of a transgenic mouse model for the sterile-feral concept.

There are many genes known to have adverse effects on fertilisation, development or reproduction in mice. These genes can be readily identified through literature and database searches (Medline, mouse knock out database, Genbank etc.). These candidate genes fall mainly into the category of genes that are required for specific developmental processes during embryogenesis. Furthermore, genes that are involved in stages of fertilisation and implantation are also potential candidate genes for this fertility control technology.

Developmental stages identified as potential sterile feral construct targets are classified under one of the following general areas: fertilisation, preimplantation, post implantation (until neurulation) and organogenesis stages. The latter stages include factors such as those associated with the specification of male and female reproductive organs (Cunha et al., 1976). Proteins involved in these stages may have different roles such as morphogens, master genes, growth factors or receptors.

Genes associated with fertilisation include such factors as protein receptors or ligands required for successful fertilisation. Preimplantation genes that can be manipulated to control their gene expression and so achieve controllable fertility are also covered by this patent and include genes encoding proteins such as growth factors, signaling molecules and their receptors.

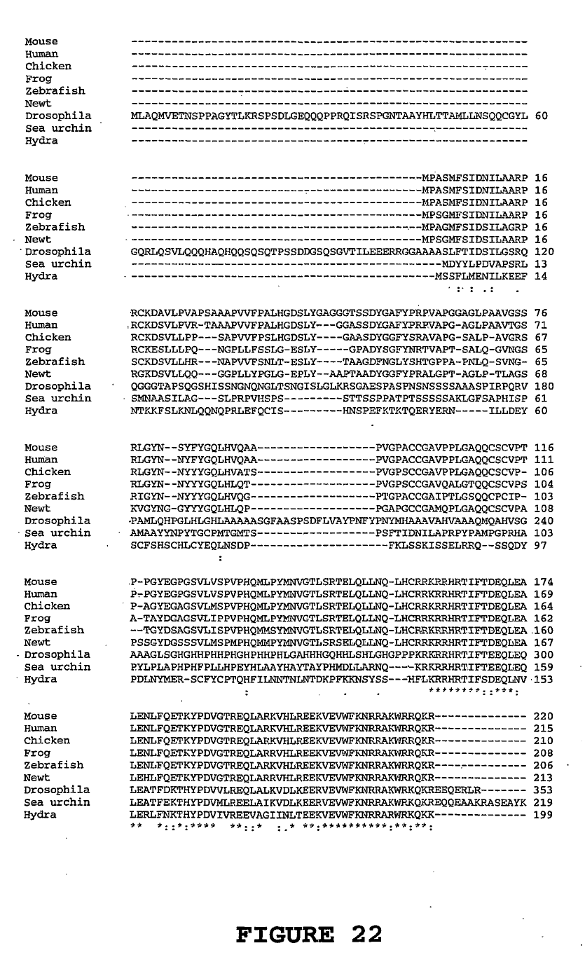

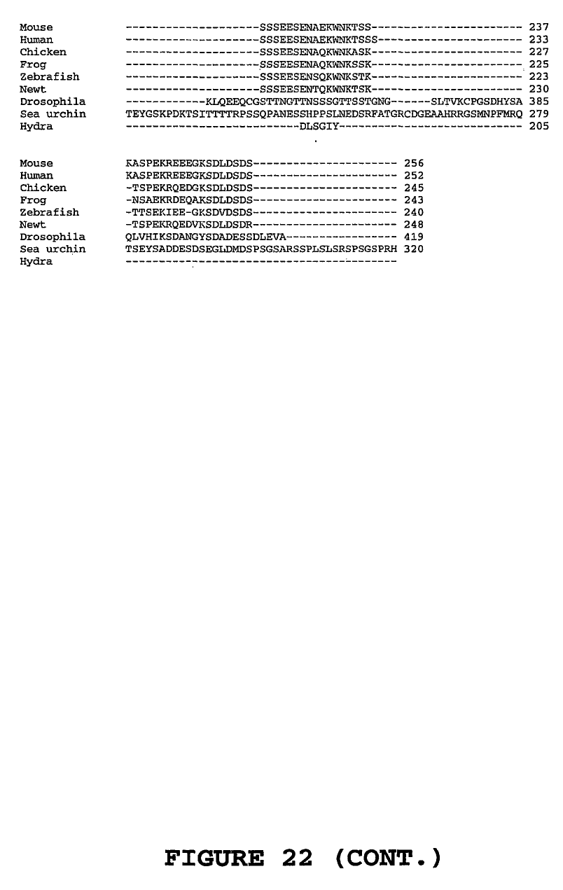

The homeobox gene goosecoid is one of the first genes to be transcribed in the organizer region of the mouse at the onset of gastrulation and RNA transcripts first appear in the dorsal mesoderm of the late blastula (Blumberg et al., 1991). The goosecoid gene is also highly conserved among different species (FIG. 22). During mouse embryogenesis, expression of the goosecoid gene takes place in two different phases. In the first phase of expression, goosecoid gene expression can be detected in the organizer between 6.4 to 6.7 days (Blum et al., 1992) and in the second phase it is detected during organogenesis from 10.5 day onwards (Gaunt et al., 1993) and expressed in some parts of head, the limbs and the ventrolateral body wall. The homozygous knockout mutation of goosecoid in the mouse leads to defects late in development of the embryos. In particular, null homozygous goosecoid embryos are born with numerous developmental defects and die within 24 hours of birth (Rivera-Perez et al., 1995). The observed phenotype is in accordance with late expression of goosecoid in normal embryos, and it has been proposed that the lack of an earlier phenotype is due to functional compensation by other orthologous genes such as gsc2.

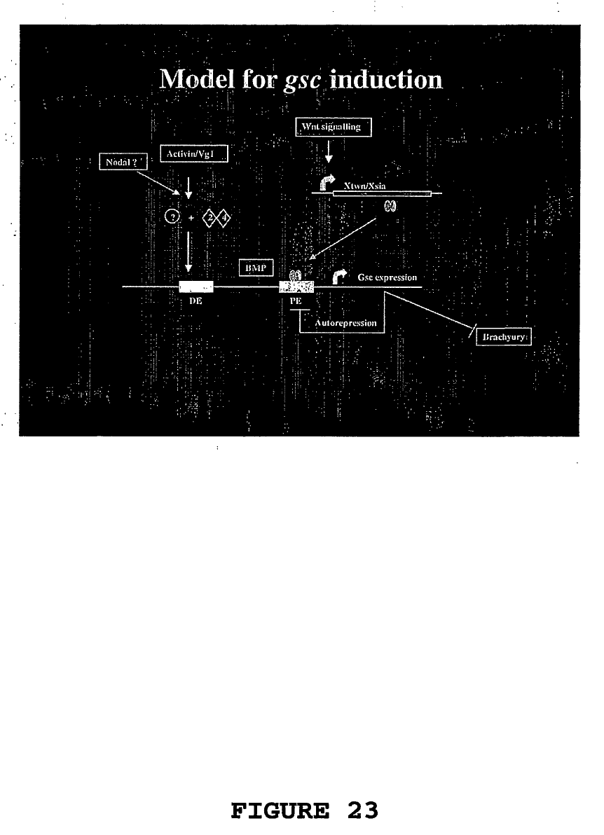

At the promoter level, molecular studies have demonstrated that expression of goosecoid in Xenopus is mediated by the combined effects of two regions of the promoter, the distal element (DE) and the proximal element (PE). The DE responds directly to dorsal mesoderm inducing signals such as activin and Vg1 (members of the TGF-β super family), whereas the PE responds indirectly to wnt signaling (McKendry et al., 1998). Sequence comparison among different species shows that these proximal and distal elements are conserved among different species and there may be a common mechanism for its activation (Blum et al., 1992). It was proposed that the DE responds directly to mesoderm inducing signals such as activin, whereas the PE responds indirectly to Wnt signaling (Laurent and Cho, 1999) (FIG. 23).

Studies involving the goosecoid promoter in mouse and other species have shown that the promoter region carrying these two elements are adequate for reporter gene activity studies. These two elements are generally located within 500 bp from the transcriptional start site.

The goosecoid gene, in the form of sterile feral constructs, can be used to demonstrate how a developmentally active gene can be manipulated to maintain its temporal and spatial gene specification under repressible promoter elements.

### EXAMPLE 12

### Cloning the Goosecoid Gene Promoter

The goosecoid promoter was amplified by PCR using BALB/c genomic DNA. Primers were designed from Mus musculus goosecoid homeobox gene, promoter sequence, of the Genbank accession number Y13151.

The primers were as follows:

| | |
|---|---|
| Forward Primer | |
| 5′-GGAGACAGGCAGTCCCGGTAGATC-3′ | SEQ ID NO: 33 |
| Reverse Primer | |
| 5′-TGGGAATTGTCCCACTCTCTGCTC-3′ | SEQ ID NO: 34 |

The PCR conditions were as follows:
95° C.×3 min, 72° C.×1 min (hotstart), 58° C.×1 min, 72° C.×1 min for 1 cycle. Then 95° C.×45 sec, 58° C.×1 min, 72° C.×1 min for 28 cycles. The reaction was completed by incubating the reaction at 72° C.×10 min and 25° C.×5 min). The PCR product for the goosecoid promoter was ligated into pGEM-T-Easy cloning vector (Promega Cat # A1360).

### EXAMPLE 13

### Selection and Construction of Reporter Plasmids for Testing Promoter Function

Reporter genes for promoter expression in mammals are available in two forms. Firstly reporter genes can be used to determine location of expression of a gene product. Examples of such commercially available reporters include the Enhanced Green Fluorescent Protein (EGFP) and Red Fluorescent Protein (RFP). Alternatively, other reporter genes can be used to quantitate relative levels of expression and include firefly luciferase (LUC+) modified for optimal expression in mammalian systems. The reporter genes EGFP and LUC+ were selected for use in testing sterile feral constructs based on the goosecoid promoter in the mouse.

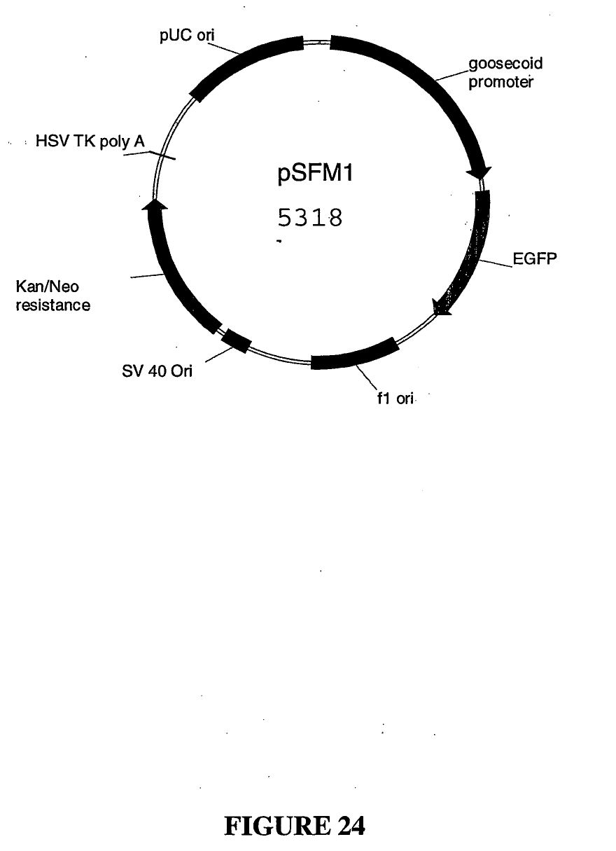

pSFM 1: goosecoid promoter expressing enhanced green fluorescent protein (FIG. 24; SEQ ID 35). The goosecoid promoter produced by PCR and cloned into pGEM-T-Easy (see above) was subcloned into the pEGFP-1 vector (Clontech Cat. # 6086-1) by digestion with EcoR1 and cloned into the EcoR1 site of the MCS of pEGFP-1. The orientation of the goosecoid promoter was confirmed by both restriction enzyme mapping and sequencing.

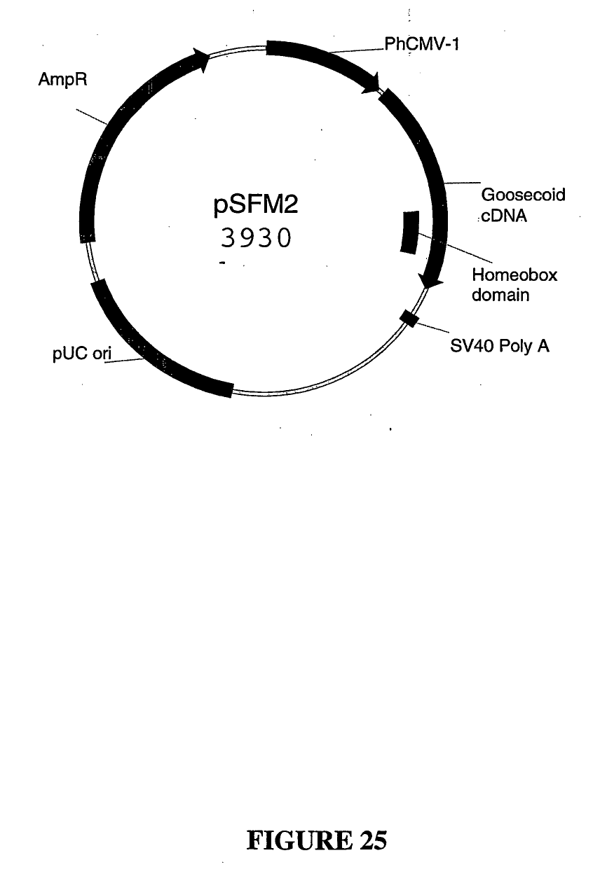

pSFM 2: goosecoid cDNA in pTRE (FIG. 25; SEQ ID 36). A goosecoid cDNA equivalent was prepared from a goosecoid genomic DNA clone. The goosecoid DNA clone was prepared by PCR using BALB/c mouse genomic DNA. Primers were designed from the published sequence of goosecoid (Genbank Accession # M85271). The goosecoid gene coding region is comprised of 3 exons. PCR primers were designed to produce each of the exons individually and were cloned into bacterial plasmid vectors using standard molecular biology techniques. The cDNA for goosecoid was then reconstructed by tandemly ligating the individual exons together to form a new clone. The exons can also be joined in other orientations to encode for various combinations of dsRNA or antisense of the goosecoid RNA.

The Primers used were designed from the entire coding region of the genomic DNA (Sequence locations referred to goosecoid Genbank Accession Number=M85271) and were:

Design of PCR primers to amplify goosecoid exons 1, 2, 3. exon 1 (bp 296-650); exon 2 (bp 1159-1418); exon 3 (bp 1765-1920):

SEQ ID NO: 37

Exon 1 forward (bp 296-316)
5′-GGTTAAGCTTATGCCCGCCAGCATGTTCAGC-3′

SEQ ID NO: 38

Exon 1 reverse (bp 631-650)
5′-GCGGGGCCCTCGTAGCCTGGGGGCGTCGGGACGCAG-3′

SEQ ID NO: 39

Exon 2 forward (bp 1165-1183)
5′-CGAGGGCCCCGGTTCTGTACT-3′

SEQ ID NO: 40

Exon 2 reverse (bp 1398-1418)
5′-TTTGAGCTCCACCTTCTCCTCCCGAAG-3′

SEQ ID NO: 41

Exon 3 forward (bp 1765-1785)
5′-GTCTGGTTTAAGAACCGCCGA-3′

SEQ ID NO: 42

Exon 3 reverse (bp 1900-1920
5′-GGAATTCTCAGCTGTCCGAGTCCAAATC-3′

Three exons were amplified by PCR using the above primers and the following conditions;
95° C.×2 min, 40° C.×30 sec, 72° C.×45 sec for 1 cycle. Then 95° C.×30 sec, 40° C.×30 sec, 72° C.×45 sec for 30 cycles. The reaction was stopped by incubation at 72° C.×10 min and 25° C.×5 min.

Goosecoid exon 1-3 PCR products were cloned into Promega (Cat # A1360) pGEM-T-Easy cloning vectors. These clones were named pME 1, pME 2 and pME 3 for exon 1-3 in pGem-T-Easy respectively.

The strategy for producing the equivalent clone for the complete goosecoid cDNA coding region was as follows:
pME 2 was cut with ApaI and religated, to remove the EcoR1 site. Pfu polymerase PCR of clone pME 3 was undertaken using the primers and conditions for exon 3 as described above. This generated a blunt-ended fragment which was then digested with EcoRI. Following religation of pME2 (see step 1 above) with EcoIcR1. Ligated together pME2 from (3) and digested PCR product from (2) to produce pME 4.

Cut pME 1 with HindIII and then partial digest with ApaI (band size 370 bp, external ApaI site).

Cut pME 4 with ApaI, followed by EcoR1.

Cut pBluescript SK− with HindIII followed by EcoRI Ligated (7) above with pME 4 product and pME 1 product to produce the complete goosecoid cDNA coding region. This clone was confirmed by sequencing and designated pCMH142 (SEQ ID 43).

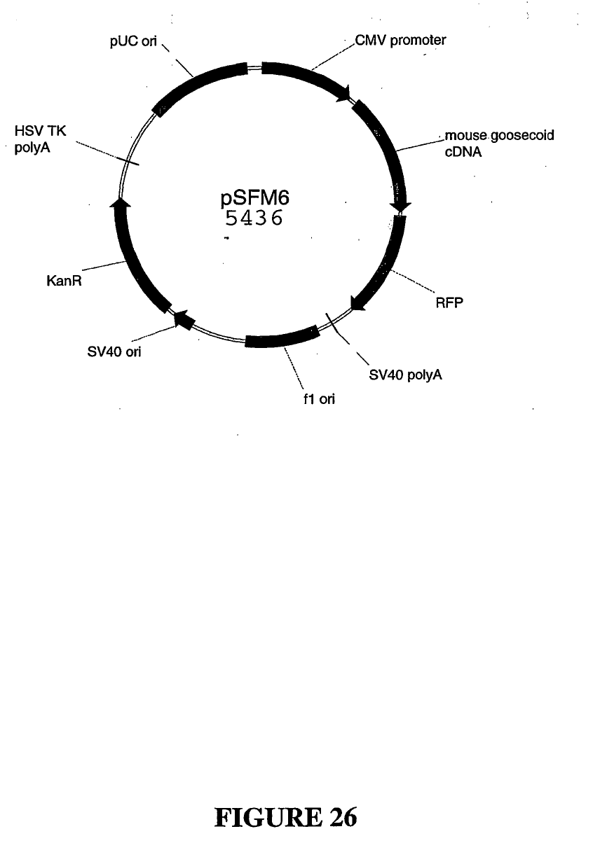

pSFM 6: Goosecoid promoter expressing goosecoid cDNA fused to red fluorescent protein (FIG. 26). A 0.9 kb PCR fragment containing the full coding sequence of mouse goosecoid was amplified from pCMH142 using two PCR primers:

| | |
|---|---|
| gsc F4 - | |
| 5′-TTAAGCTTGCCACCATGCCCGCCAGCATGT-3′ | SEQ ID 44 |
| gsc R4 - | |
| 5′-TTGGATCCGCGCTGTCCGAGTCCAAATC-3′ | SEQ ID 45 |

These primers produced a goosecoid-containing fragment where the TGA stop codon was replaced with an alanine codon. The PCR primers were also used to introduce a HindIII site upstream of the ATG start codon and a BamHI site downstream of the alanine codon. This fragment was restricted with HindIII and BamHI and then inserted into the plasmid pDSRed1-N1 (Clontech 6921-1) cut with HindIII and BamHI in order to generate pSFM 6 (SEQ ID 46).

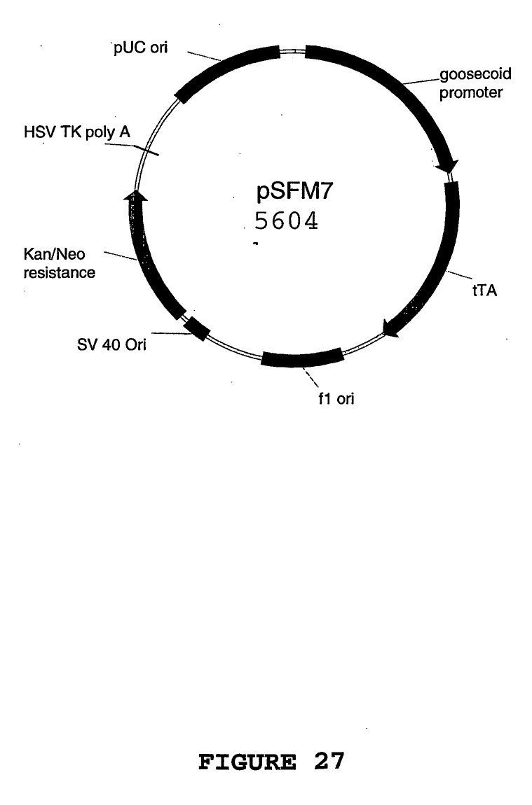

pSFM 7: Mouse goosecoid promoter expressing the tetracycline transactivator protein tTA (FIG. 27). SEQ ID 47.

The goosecoid tetracycline dependent transactivator plasmid was constructed by replacing EGFP of pSFM 1 with the 1008 bp coding region region (Genbank accession # U89930 bp 774-1781) of the tet-responsive transcriptional activator (tTA) from the pTET-OFF plasmid (Clontech, Cat # K1620-A). The tTA coding region was amplified by PCR using Pfu polymerase, restricted by Age1 and EcoR1 and cloned into pSFM 1 to produce pSFM 7.

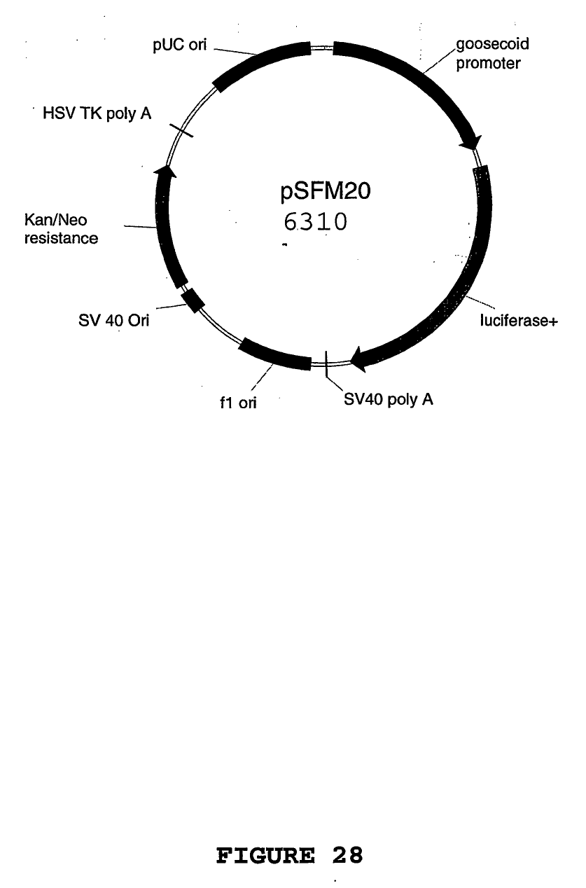

pSFM 20: goosecoid promoter expressing luciferase+ protein (FIG. 28). SEQ ID 48.

A 0.7 kb (NotI end-filled with Klenow+BamHI) fragment coding for green fluorescent protein region from pSFM1 was replaced with 1.6 kb (XbaI end filled with Klenow enzyme+BamHI) luciferase+ coding fragment derived from pXP1-G (Promega E1751).

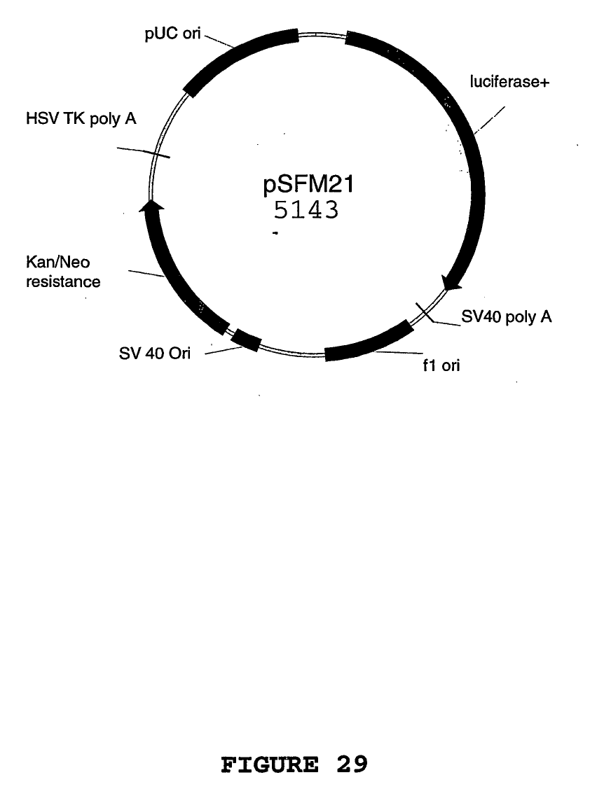

pSFM 21: Promoterless luciferase+ (FIG. 29). SEQ ID 49.

A 1.6 kb luciferase coding EcoRI fragment was deleted from pSFM 20.

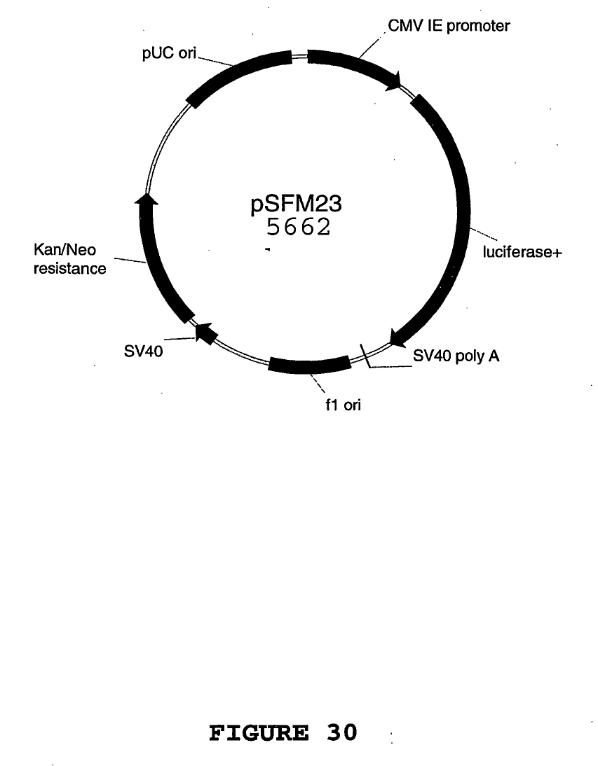

pSFM 23: pCMV promoter expressing luciferase+ (FIG. 30). SEQ ID 50.

A 1.6 kb (SacI+StuI) luciferase+ coding fragment of pSFM 20 was cloned into pEGFP-N1 (Clontech 6085-1) cut with SacI+StuI.

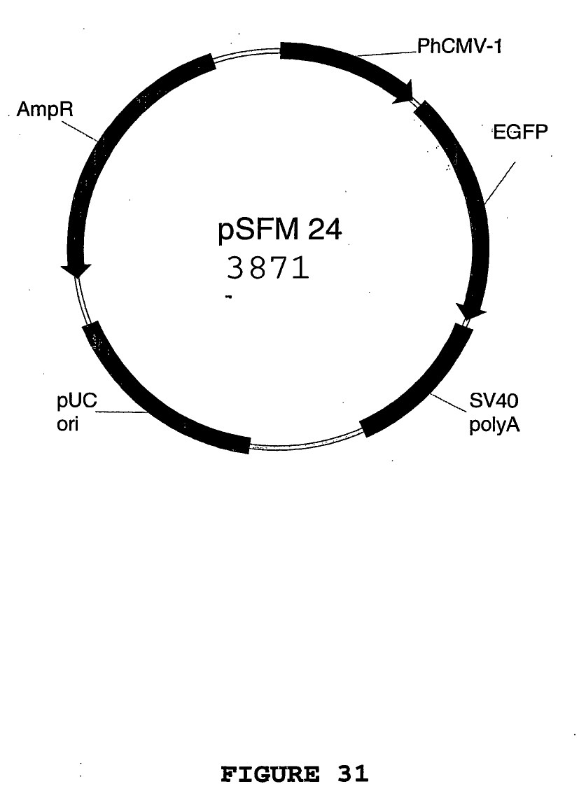

pSFM 24: Equivalent to the tet-responsive enhanced green fluorescent protein expression vector pTRE-EGFP (Clontech 6241-1)(FIG. 31) SEQ ID 51.

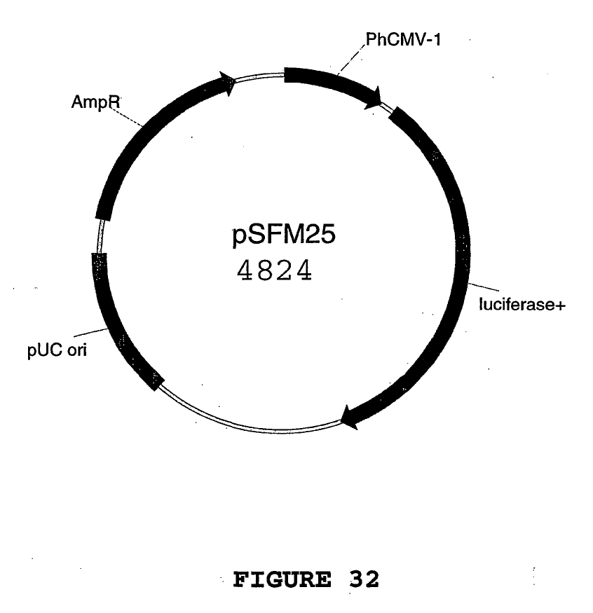

pSFM 25: Tet-responsive expression vector pTRE-luciferase+ (FIG. 32). SEQ ID 52.

A 0.77 kb SalI+XbaI EGFP containing fragment of pSFM 24 was replaced by a 1.7 kb SalI+XbaI luciferase+ containing fragment derived from pXP1-G (Promega).

### EXAMPLE 14

### Selection of Mammalian Cell Lines

Mouse goosecoid was selected to demonstrate whether a developmental gene can be tightly regulated in the form of sterile feral constructs in mammalian cell lines. Most of the mainpulations using sterile feral constructs based on goosecoid were therefore carried out in the mouse embryo cell lines P19 teratocarcinoma since it has been shown previously that the mouse goosecoid gene product is constitutively expressed in P19 teratocarcinoma cell lines. NIH/3T3 cells (in which goosecoid gene expression is absent) were used as controls.

In addition goosecoid reporter constructs were tested in non-transformed mouse primary embryonic fibroblasts. These cells display monolayered, anchorage dependent and contact inhibited growth in tissue culture. Using transient transfection with reporter and other plasmid constructs (reporters and blockers) the observed effects on these plasmids is expected to reflect the anticipated effect in the whole organism.

Chromatin structure surrounding the inserted gene is also likely affect the pattern of regulation of gene expression and so the choice of stable cell lines for gene expression is essential. For example, it is known that transfected DNA does not display the same accessibility to transcriptional factors as chromosomal DNA (Archer et al., 1992). Another important factor to consider is that the goosecoid promoter contains only 1.1 kb upstream to the transcription start site leading to potential restriction of access by nuclear and other transcriptional factors by surrounding DNA sequences and chromatin structure.

All cell lines were obtained from American Type Culture Collection unless otherwise stated. These are P19 teratocarcinoma cells (ATCC number CRL-1825) and NIH/3T3 cells (ATCC number CRL-1658).

For transient transfection assays, P19 cells were cultured on gelatinized dishes in DMEM supplemented with 10% fetal bovine serum. Cells (0.3 million per well in 6-well cluster plates) were transfected with 5 μg reporter plasmid using transfection reagent ‘Geneporter’ from Gene Therapy Systems according manufacturer's recommendation.

Stably integrated P19 clones were obtained by using BioRad Gene Pulser II electroporation system. 30 μg DNA electroporated into 10 million cells under following conditions 960 μF and 0.16 kV in a 0.4 cm cuvette (0.4 kV/cm). The next day normal media were replaced with appropriate selection media (300 μg/ml G418).

Reverse transcriptase polymerase chain reaction (RT-PCR) was used to confirm that the goosecoid gene is actively expressed in P19 cell lines with the goosecoid specific primers exon 2 forward (SEQ. ID 39) and exon 3 reverse (SEQ ID 42):

RT-PCR

cDNA was synthesized in a 50 μl reaction using 100 ng of poly(A) RNA extracted from various tissues and cell lines. The RNA was heated with a mixture of random 6 base pair and oligo(dT) primers for 5 min at 65° C. and cooled to room temperature for 10 min. Reverse transcription was performed at 37° C. for 1 h after adding 5 μl. 10×RT buffer (Promega), 20 U RNase inhibitor (Promega), 2 μl of 0.1 mM dNTPs and 50 U MMLV reverse transcriptase. The cDNA mixture was then heated for 5 min at 90° C. and stored at −20° C. until needed.

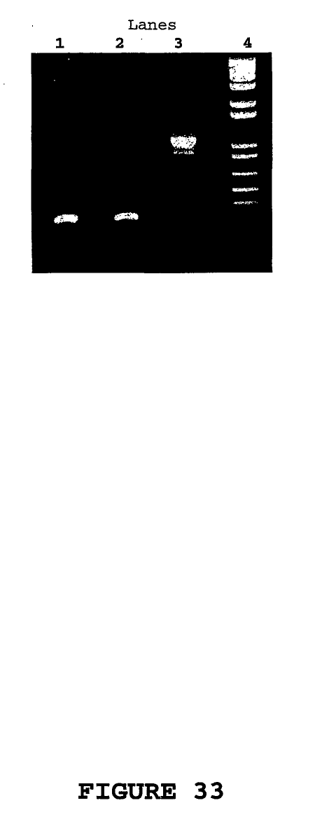

RT-PCR was conducted using 2 μl of cDNA in a 50 μl final reaction using goosecoid specific primers (FIG. 33). By comparison, RT-PCR amplification on NIH/3T3 cells gave negative results for goosecoid. In both cells, RT-PCR of a general housekeeping gene GADPH gave positive bands. In addition GFP expression from P19 cells containing the reporter plasmid pSFM 1 stably integrated was unaffected by repeated passaging or freezing and thawing.

In order to measure the activity of the goosecoid gene, a cell culture system was developed that responds to tetracycline repression and permits the measurement of gene activity using both fluorescence reporters.

Fluorescent and transmitted light images were acquired using a CCD camera with a microscope. Fluorescence filter sets had an excitation wavelength of 480 nm, dichroic cut-on filter at 505 nM and an emission filters at 535 nM and 605 nM. The luminescence assays were conducted by using a dark 96 well plate was done by Victor2 from Wallac or by Topcount NXT from Can berra Packard.

P19 cells were transiently transfected in 6 well plates with pSFM 20 (goosecoid promoter-luciferase), pSFM 21 (promoterless luciferase) and pSFM 23 (CMV promoter-luciferase) using Gene Porter. Cells were harvested at various times post-tranfection and assayed for luciferase activity using a Promega kit (Cat. # E1501) in a Top Count NXT luminometer.

Table 19 shows the luciferase activities of promoter reporter constructs shown in counts per second (cps) of transiently transfected in P19 cells.

TABLE 19

| Hours | pSFM 21 | pSFM 23 | pSFM20 |
|---|---|---|---|
| 24 | 254 | 78778 | 1263 |
| 48 | 604 | 145403 | 3707 |
| 72 | 252 | 49936 | 1692 |

Maximum luciferase activity was observed 48 hours post-transfection for all plasmids. Luciferase activity from the goosecoid promoter construct (pSFM 20) was 6 fold higher compared to the promoterless construct (pSFM 21). CMV driven luciferase activity (pSFM 23) was 200-300 fold higher than for the promoterless luciferase (pSFM 21). Therefore 48 hours post transformation was selected for optimal detection of luciferase expression.

Selection of a P19 cell line stably integrated with a goosecoid-dependent TET-OFF transactivator P19 cells were electroporated with pSFM 7 (Goosecoid promoter-TET/OFF) linearised with ApaLI and selected for stable integration.

Table 20 showes the luciferase activities of pSFM 25 (TRE luciferase+) shown in counts per second (cps) of transiently transfected in P19-pSFM 7 cells.

TABLE 20

| | pSFM 25 | pSFM 25 |
|---|---|---|
| Clone number | without doxycycline | with doxycycline |
| 9 | 582529 | 54858 |
| 12 | 417268 | 4396 |
| 29 | 616604 | 48260 |
| 32 | 272888 | 19548 |
| 46 | 703470 | 8013 |

From 100 clones, one clone (46) was selected which demonstrated the highest luciferase activity when transiently transfected with the reporter plasmid pSFM 25 (TRE-luciferase+). Addition of doxycycline at 1 μg/ml reduced luciferase activity from pSFM 25 in this clone by 90 fold. This clone, containing stably integrated pSFM 7 was therefore designated P19-pSFM 7 and used for further testing.

Reporter plasmids pSFM 20 (goosecoid promoter luciferase+), pSFM 21 (promoterless luciferase+), pSFM 23 (CMV promoter luciferase+) and pSFM 25 (TRE luciferase+) were transiently transfected into either P19 or P19-pSFM 7 (Goosecoid TET/OFF) cells to test the effectiveness of the TET-OFF genetic switch driven by goosecoid promoter. Table 21 shows the luciferase activities of transient transfection of reporter plasmids in P19 and P19-pSFM 7 cell lines.

TABLE 21

| | |
|---|---|
| Pl9-pSFM 7 cells | P19 cells |

| Plasmids | Average | Fold | Average | Fold |
|---|---|---|---|---|
| pSFM 20 | 365 | 6 | 610 | 5 |
| pSFM 21 | 60 | 1 | 121 | 1 |
| pSFM 23 | 16031 | 267 | 44491 | 367 |
| pSFM 25 | 368 | 6 | 183 | 1.5 |

P19-pSFM 7 but not the P19 cells show a 6 fold increase in luciferase+ reporter activity when transfected with pSFM 25 compared to the promoterless plasmid pSFM 21. This increase is comparable to the increase seen when the cells are transfected with plasmids containing the luciferase driven by the goosecoid promoter (pSFM 20). Therefore the P19-pSFM 7 cell line can be used to drive expression through pTRE plasmids to the same level as plasmids driving expression from the goosecoid promoter directly.

### EXAMPLE 15

### Construction and Testing of Blocker Plasmids

Antisense and double stranded blockers specific for goosecoid were constructed.

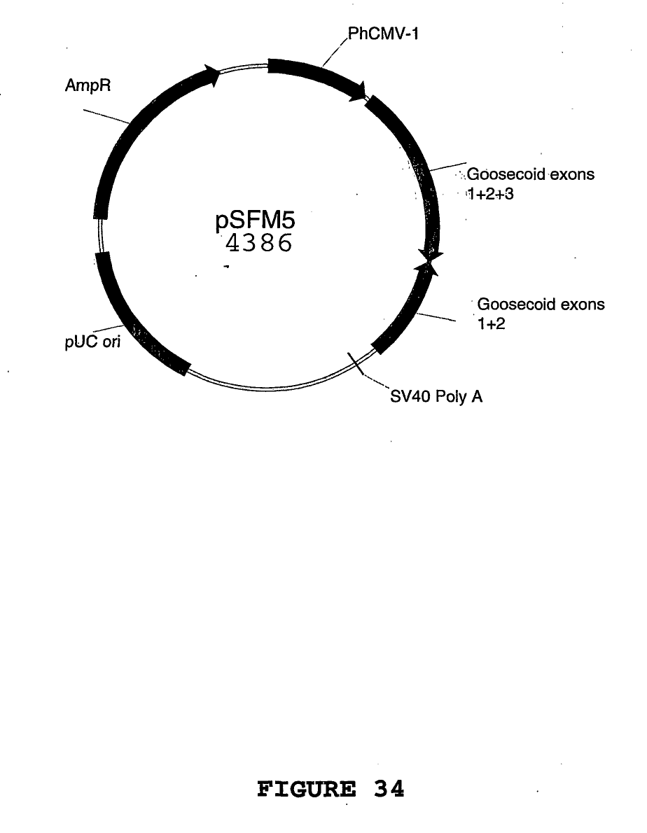

pSFM 5: Tet-responsive expression vector pTRE-goosecoid double strand RNA (FIG. 34). SEQ ID 57.

pSFM5 was derived from pSFM 2 and pSFM 9. A 0.48 kb PstI+BamHI fragment of pSFM 9 was inserted into a 3.9 kb PstI partial+BamHI fragment of pSFM 2 to produce pSFM 5.

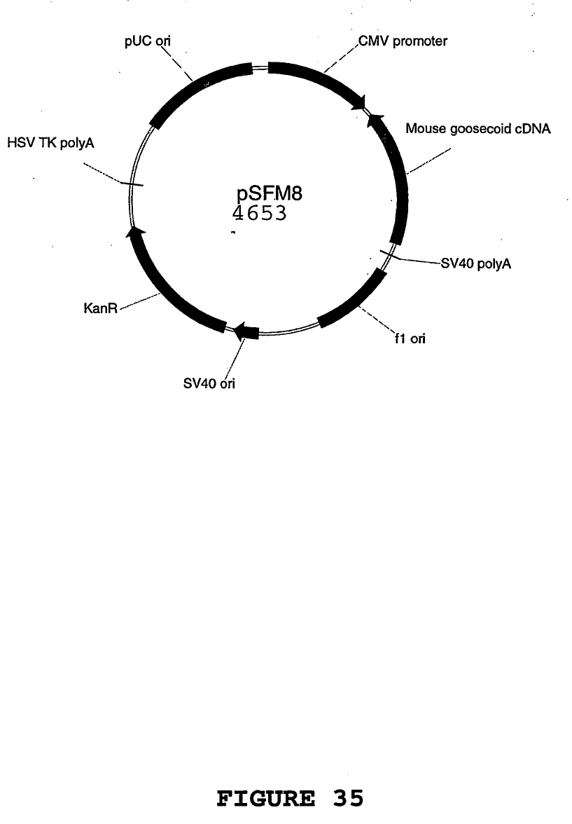

pSFM 8: pCMV promoter expressing goosecoid antisense RNA (FIG. 35). SEQ ID 58.

A 0.8 kb EcoRI+KpnI fragment of pSFM 9 containing the goosecoid cDNA was inserted into pdsRED-N1 (Clontech 6921-1) cut with KpnI+EcoR1. This clone was then cut with SmaI+HpaI to remove the RFP and religated to produce pSFM 8.

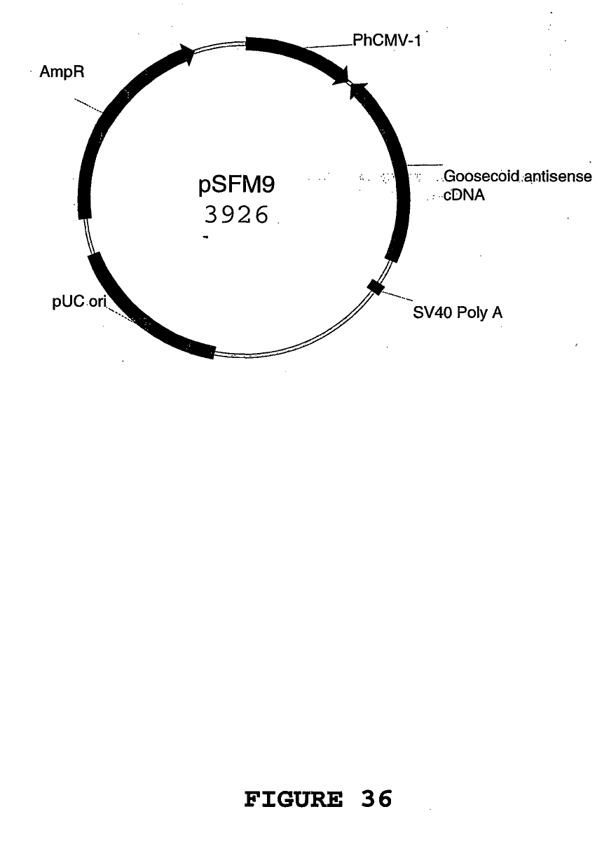

pSFM 9: Tet-responsive expression vector pTRE-goosecoid antisense RNA (FIG. 36). SEQ ID 59.

A 0.78 kb HindIII Klenow end-filled+EcoRI fragment of pCMH142 was cloned into pTRE cut with BamHI end-filled with Klenow+EcoRI.

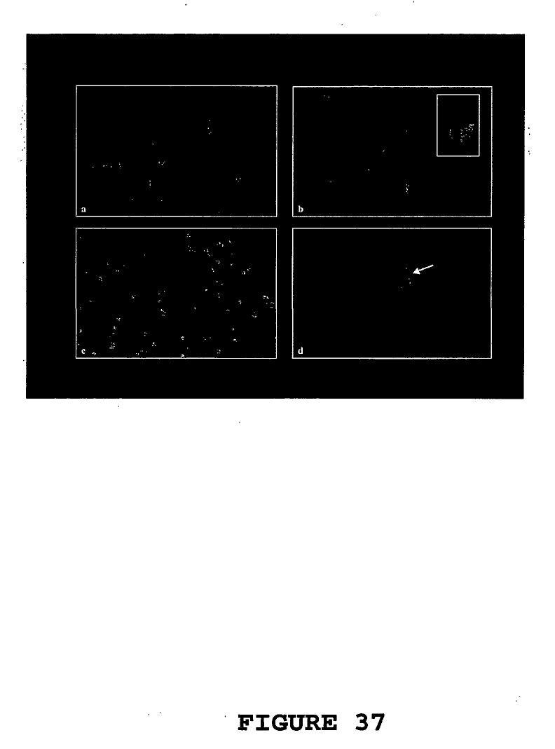

The first stage for testing blocker constructs is to set up an appropriate cell system to detect expression of reporter constructs. Initially, either pdsRED-N1 (CMV promoter RFP), pSFM 6 (CMV promoter goosecoid cDNA fused to RFP) or pSFM 24 (TRE EGFP) were transfected into P19-pSFM 7 cells to test the expression patterns of the EGFP, RFP and goosecoid-fused to RFP proteins (FIG. 37). These tests show that RFP is expressed in the cytoplasm when driven from a CMV promoter (FIG. 37,B). When goosecoid is fused to RFP and driven from a CMV promoter however, the RFP signal is now detected in the nucleus (FIG. 37C,D), whereas the EGFP is expressed in the cytoplasm of the same cells when expressed through the TRE promoter (FIG. 37D). This shows therefore, that goosecoid is efficiently transferred to the nucleus when fused to the reporter gene RFP and that this system can be used to test co-transfected blocker plasmids against goosecoid. In these cases, RFP expression fused to goosecid in the nucleus is expected to be inhibited in the presence of an appropriate blocker.

In order to assess various antisense and dsRNA blockers, pSFM 6 (CMV promoter goosecoid fused to RFP) was transiently cotransfected into the P19-pSFM 7 (Goosecoid promoter TET/OFF) cells along with either pSFM 5 (TRE promoter dsRNA goosecoid), pSFM 8, (CMV promoter antisense goosecoid), pSFM 9 (TRE promoter antisense goosecoid) or pSFM 24 (TRE promoter EGFP). In these cases, significant difference could not be detected between the various treatments in either the intensity or number of cells expressing RFP in the nucleus. There are several potential reasons for the absence of RNA blocker effects. First, antisense and dsRNA blockers may not be expressed at levels high enough to effectively interfere with the target mRNA molecules. Secondly, there may be cellular mechanisms in mammals that recognize and interfere with such constructs. Thirdly, the RNA inhibitory molecules may not be able to access and block the RNA target.

The goosecoid gene, in the form of sterile feral constructs, was tested in mammalian cells to demonstrate whether plasmids DNA coding for SF blockers have effect any effect on blocking goosecoid expression. We have demonstrated the methods for producing stably integrated cell lines and the testing of blocker constructs based on goosecoid dsRNA and goosecoid anti-sense. Our results suggest that post-transcriptional silencing through double strand RNA is unlikely to be very effective in mice. We therefore conclude that either the system described here is insufficiently sensitive to detect RNA interference using the current blockers or that these inhibitors are relatively ineffective in the P19 mammalian cell line. Nevertheless, small effects in cell culture can translate into severe phenotypic abnormality when introduced into mice.

By contrast, over-expression and mis-expression of genes leading to developmental abnormalities has been demonstrated in mice (Zwijsen et al., 1999; Goodrich et al., 1999). It can be reasonably expected therefore that sterile feral blockers that cause over-expression or mis-expression of developmental genes through at tetracycline repressible system will succeed. However, sense constructs cannot be easily tested using reporter systems. It is necessary to stably introduce such constructs into embryonic stem (ES) cells and produce transgenic mice to evaluate the extent to which development can be disrupted.

### EXAMPLE 16

### Production of Transgenic Mice Using the Goosecoid Promoter

By using the goosecoid gene promoter (or similar) to drive expression of known proteins critical to early embryogenesis a transgenic mouse can be made. Candidate sense blockers for expression from the goosecoid promoter are gene products that are critical for development in the mouse and are also normally expressed in the embryo during gastrulation at the same time as the goosecoid gene product. Two other proteins, Chordin and Noggin, are known to expressed within the same embryonic region at times and locations similar to that of goosecoid (Bachiller et al., 2000). In particular, Chordin is expressed in the same region as goosecoid at embryonic stage TS 11 in the primitive streak and node.

Double knock-out mice for Chordin and Noggin have been produced and these show severe phenotypic defects in the prosenchephalon. Both of these proteins are therefore essential for successful development in the mouse. These two genes are antagonisers of another gene product, BMP-4, which is expressed in the region adjacent to the primitive streak. Together, these three gene products contribute to the anterior/posterior structural features of the developing mice. Therefore, misexpression of BMP-4 using the goosecoid promoter, within the primitive streak, where Noggin and Chordal are expressed, will interefere with the balance between these gene products and be expected to produce a phenotype that will match the double knock-out for Chordin and Noggin. Many other developmental genes, particularly those involved with early embryogenesis could be misexpressed in a similar manner.

The following process can be used to generate a transgenic mouse line expressing repressible developmentally regulated blockers. Gene targeting in mice is regularly achieved using two different methods. One is by oocyte injection and the other is through gene insertion into embryonic stem cells. The embryonic stem cell method is the most preferred for manipulations using the goosecoid gene since this gene is usually activated following removal of leukemic inhibitory factor (a factor used to maintain the cells in undifferentiated state) from the culture medium (Savatier et al., 1996). Testing for effectiveness of reversible blockers on goosecoid expression in cell cuture can therefore be tested in embryonic stem cells before being transferred into mouse but not in system using directly injected oocytes.

The manipulations and production of repressibly steile transgenic mice is readily achievable to those practiced in the art (Hogan et al., 1994). This involves the following steps:

Transfection, stable integration and selection of embryonic stem cells with a sterile feral construct consisting of the goosecoid promoter driving expression of tTA (Tet-Off) such as pSFM 7 (SEQ ID NO:48).

Transfection, stable integration and selection of the teracycine dependent effector construct consisting of the TRE (Tet-reponsive promoter) driving expression of one of the following: goosecoid antisense or dsRNA in constructs such as pSFM 9 (SEQ ID NO:59) and pSFM 5 (SEQ ID NO:57) or the cDNA for genes essential for development in the embryo around the time of primitive streak formation (such as BMP-4).

### Conclusions

One type of “sterile feral” construct encompassed by the present invention consists of three components, a developmental or constitutive promoter, a gene blocker sequence, and a repressible promoter from Clontech™'s commercially available Tet-Off system. The developmental or constitutive promoter functions to drive expression of Tet-Off represser protein (tTA, Clontech™) which binds to the tet responsive element (TRE-CMVₘᵢₙ, Clontech™) that in turn drives expression of the gene blocker sequence. Expression of the blocker DNA sequence results in production of either antisense or double stranded mRNA to ultimately knock-out function of the target gene or mis-expression of a sense sequence, that causes distorted development and embryo death. Correct function of the sterile feral construct requires that functions of both the developmental promoter and the target gene are confined to either oogenesis or embryogenesis. This can be achieved optimally by using a stage-specific promoter, though it can also be achieved through use of a developmental blocker who's effects are also spatio-temporally confined to early embryogenesis. Repression of the blocker sequence function is accomplished through exposure to tetracyline which prevents the binding of the tTA to the TRE-CMVₘᵢₙ.

### REFERENCES

Archer, T. K., P. Lefebvre, et al. (1992). Transcription factor loading on the MMTV promoter: a bimodal mechanism for promoter activation [published erratum appears in Science 1992 Apr. 10;256(5054):161]. Science 255(5051): 1573-6.
Ausubel, F. M., Brent, R., Kingston, R. E., Moore, D. D., Sridman, J. G., Smith J. A. and Struhl, K. (Eds.; 1996). Current protocols in molecular biology. John Wiley and Sons Inc, USA. Vol 1-3.
Bachiller, D., J. Klingensmith, et al. (2000). The organizer factors Chordin and Noggin are required for mouse forebrain development. Nature 403 (6770): 658-61.
Blum, M., S. J. Gaunt, et al. (1992). Gastrulation in the mouse: the role of the homeobox gene goosecoid. Cell 69(7): 1097-106.
Blumberg, B., C. V. Wright, et al. (1991). Organizer-specific homeobox genes in Xenopus laevis embryos. Science 253(5016): 194-6.
Cormack B P, Valdivia R H, Falkow S (1996) FACS-optimised mutants of the green fluorescent protein (GFP). Gene 173:33-38.
Cunha, G. R. (1976). Stromal induction and specification of morphogenesis and cytodifferentiation of the epithelia of the Mullerian ducts and urogenital sinus during development of the uterus and vagina in mice. J Exp Zool 196(3): 361-70.

Dale, L., Howes, G., Price, B. M. J. and Smith J. C. (1992) Bone morphogenetic protein 4: a ventralizing factor in early Xenopus development. Development 115:573-585
DeRobertis, E. M. and Sasai, Y (1996). A common plan for dorsoventral patterning in bilateria. Nature 380:37-40. Dick et al (1999). Smad1 and Smad5 have distinct roles during dorsoventral patterning of the zebrafish embryo. Developmental Dynamics 216:285-298.
Driever et al (1996). A genetic screen for mutations affecting embryogenesis in zebrafish. Development. 123:37-46
Driever, W. and Rangini, Z. (1993). Characterization of a cell line derived from zebrafish (Bracydanio rerio) embryos. In Vitro (Cell. Dev. Biol. 29A: 749-754. factor encoded by the short gastrulation gene. Genes Dev 8:2602-2616.
Ferguson, E. L. and Anderson, K. V. (1992). Decapentaplegic acts as a morphogen to organize dorsal-ventral pattern in the Drosophila embryo. Cell 71:451-461.
Fire, A., Xu, S., Montogomery M. K., Kostas, S. A. Driver, S. E. and Mello C. C. (1998). Potent and specific genetic interference by double-stranded RNA in Caenorhabditis elegans. Nature.391:806-811.
Francois, V., Solloway, M., O'Neill, J. W., Emery, J. and Bier, E. (1994) Dorsal-ventral patterning of the Drosophila embryo depends on a putative negative growth
Gaunt, S. J., M. Blum, et al. (1993). Expression of the mouse goosecoid gene during mid-embryogenesis may mark mesenchymal cell lineages in the developing head, limbs and body wall. Development 117(2): 769-78.
Goodrich, L. V., D. Jung, et al. (1999). Overexpression of ptc1 inhibits induction of Shh target genes and prevents normal patterning in the neural tube. Dev Biol 211(2): 323-34.
Gossen, M. & Bujard, H. (1992) Tight control of gene expression in mammalian cells by tetracycline responsive promoters. Proc. Natl. Acad. Sci. USA 89:5547-5551.
Gossen, M., Freundlieb, S., Bender, G., Muller, G., Hillen, W. & Bujard, H. (1995) Transcriptional activation by tetracycline in mammalian cells. Science 268:1766-1769.
Hafter et al (1996). The identification of genes with unique and essential functions in the development of the zebrafish, Danio rerio. Development. 123:1-36.
Hammerschmidt, M., Serbedzija, N. and McMahon, A. P. (1996) genetic analysis of dorsoventral pattern formation in the zebrafish: requirement of a BMP-like ventralizing activity and its dorsal represser. Genes Dev. 10:2452-2461.
Hild et al (1999). The snad5 mutation somitabun blocks Bmp2b signalling during early dorsoventral patterning of the zebrafish embryo. Development 126: 2149-2159.
Hogan, B., R. Beddington, et al., Eds. (1994). Manipulating the Mouse Embryo, Cold Spring Harbor Laboratory Press.
Holley, S. A., Jackson, P. D., Sasai, Y., Lu, B., De Robertis, E. M., Hoffmann, F. M., and Ferguson, E. L. (1995) A conserved system for dorsal-ventral patterning in insects and Animals involving sog and chordin. Nature 376: 249-253
Izant J G, Weintraub H (1984). Inhibition of thymidine kinase gene expression by anti-sense RNA: a molecular approach to genetic analysis. Cell 36:1007-1015.
Jefferson, R. A. (1987). Assaying chimeric genes in plants: The GUS gene fusion system. Plant Mol. Biol. Rep. 5: 387-405.
Jones, C. M., Dale, L., Hogan, B. L. M., Wright C. V. E. and Smith, J. C. (1996) Bone morphogenetic protein-4 (BMP4) acts during gastrula stages to cause ventralization of Xenopus embryos. Development 122:1545-1554.
Kishimoto, Y., Lee, K. H., Zon, L., Hammerschmidt, M. and Schulte-Merker, S. (1997). The molecular nature of zebrafish swirl: BMP2 function is essential during early dorsoventral patterning. Development. 124:4457-4466.
Kistner A, Gossen M, Zimmermann F, Jerecic J, Ullmer C, Lubbert H, and Bujard H. (1996) Doxycycline-mediated quantitative and tissue-specific control of gene expression in transgenic mice. Proc Natl Acad Sci USA 93:10933-10938.

Laurent, M. N. and K. W. Cho (1999). Bone morphogenetic protein antagonism of Spemann's organizer is independent of Wnt signaling. Dev Biol 206(2): 157-62.
Lemaire, P. and Yasuo H. (1998). Developmental signalling: a careful balancing act. Curr Biol.8:228-231
McKendry, R., R. M. Harland, et al. (1998). Activin-induced factors maintain goosecoid transcription through a paired homeodomain binding site. Dev Biol 204(1): 172-86. mechanisms of dorsal-ventral patterning. Development 121:4319-4328
Mishihina, Y., Susuki, A., Ueno. N. and Behringer, R. R. (1995). BMPr encodes a type I bone morphogenetic protein receptor that is essential for gastrulation during mouse embryogenesis. Genes and Dev. 9:3027-3037.
Miya, T., Morita, K., Suzuki, A., Ueno, N. and Satoh, N. (1997). Functional analysis of an ascidian homologue of Animal BMP-2/BMP-4 suggests its role in
Piccolo, S., Sasai, Y., Lu, B. and DeRobertis, E. M. (1996). A possible molecular mechanism for Spemann organizer function: Inhibition of ventral signals by direct binding of Chordin to BMP-4. Cell 85:589-598.
Rivera-Perez, J. A., M. Mallo, et al. (1995). Goosecoid is not an essential component of the mouse gastrula organizer but is required for craniofacial and rib development. Development 121(9): 3005-12.
Sambrook, J., Fritsch, E. F. and Maniatis, T. (eds.; 1989). Molecular Cloning A Laboratory Manual, 2nd Ed., Cold Spring Harbor Laboratory Press. Vol 1-3.
Savatier, P., H. Lapillonne, et al. (1996). Withdrawal of differentiation inhibitory activity/leukemia inhibitory factor up-regulates D-type cyclins and cyclin-dependent kinase inhibitors in mouse embryonic stem cells. Oncogene 12(2): 309-22.
Schmidt, J., Francois, V., Bier, E. and Kimelman, D. (1995). Drosophila short gastrulation induces an ectopic axis in Xenopus: evidence for conserved
Schulte-Merker, S., Lee, K., McMahon A. P. and Hammerschmidt, M. (1997). The zebrafish organizer requires chordino. Nature 387:862-863.
Stoffregen, D. A., Bowser, P. R. and Babish J. G. (1996). Antibacterial chaemotherapeutants for finfish aquaculture: A synopsis of laboratory and field efficacy and saftey studies. J. aquatic animal health. 8:181-207.
Suzuki Y, Yandell M D, Roy P J, Krishna S, Savage-Dunn C, Ross R M, Padgett R W, Wood W B (1999). A BMP homolog acts as a dose-dependent regulator of body size and male tail patterning in Caenorhabditis elegans. Development 126(2): 241-250 the inhibition of neural fate specification. Development.124:5149-5159
Tjian R, Maniatis T (1994). Transcriptional activation: a complex puzzle with few easy pieces. Cell. 8:77:5-8.
Waterhouse, P., Graham, M. W. and Wang, M-B. (1998). Virus resistance and gene silencing in plants can be induced by simultaneous expression of sense and antisense RNA. Proc. Natl. Acad. Sci. 95:13959-13964.
Westerfield M. (1995). The zebrafish book: University of Oregon Press.
Zwijsen, A., M. J. Goumans, et al. (1999). Ectopic expression of the transforming growth factor beta type II receptor disrupts mesoderm organisation during mouse gastrulation. Dev Dyn 214(2): 141-51.

1. A method of controlling fertility in an animal comprising the steps of:
1) stably transforming an animal cell or single celled embryo with a construct comprising:
a) a first nucleic acid molecule, which is activated in a defined spatio-temporal pattern, and which is operably linked to
b) a second nucleic acid molecule, which encodes a transactivating protein; and
c) a third nucleic acid molecule, which is operably linked to a fourth nucleic acid molecule,
wherein activation of said first nucleic acid molecule controls the expression of the second nucleic acid molecule, which in turn activates the third nucleic acid molecule, which effects the expression of the fourth nucleic acid molecule which encodes a blocker molecule which disrupts gametogenesis or embryogenesis in the animal; and
2) and growing a whole animal directly from that cell or implanting the cell into a host animal, whereby a whole animal develops from the implanted cell.

2. The method of claim 1, wherein said first or said fourth nucleic acid molecule is transiently activated or transiently affects development in a defined spatio-temporal pattern.

3. The method of claim 1, wherein each of the first, second, third and fourth nucleic acids is genomic DNA, cDNA, RNA, or a hybrid molecule thereof.

4. The method of claim 3, wherein the nucleic acid molecule is a full-length molecule, or a biologically active fragment thereof.

5. The method of claim 1, wherein the first nucleic acid molecule is a DNA molecule encoding a promoter region.

6. The method of claim 5, wherein the promoter is activated only during embryonic development and/or gametogenesis, and is crucial for completion of embryogenic development and/or gametogenesis.

7. The method of claim 5, wherein the promoter comprises the nucleotide sequence of SEQ ID NO:1, SEQ. ID NO:8, or SEQ ID NO:60.

8. The method of claim 1, wherein the second nucleic acid molecule is a cDNA molecule encoding a tetracycline-responsive transcriptional activator protein.

9. The method of claim 8, wherein said tetracycline-responsive transcriptional activator protein comprises the nucleotide sequence of SEQ ID NO:2.

10. The method of claim 1, wherein the third nucleic acid molecule encodes a repressible promoter.

11. The method of claim 10, wherein the promoter consists of a tet-responsive element (TRE) which is coupled to and tightly regulates a minimal promoter region.

12. The method of claim 11, wherein said minimal promoter region is a PminCMV promoter region that comprises the sequence of SEQ ID NO:3.

13. The method of claim 1, wherein the fourth nucleic acid molecule encodes a blocker molecule selected from the group consisting of an antisense RNA, a double-stranded RNA (dsRNA), a sense RNA and a ribozyme.

14. The method of claim 13, wherein the molecule is dsRNA or sense RNA that when mis-expressed disrupts development in a defined spatio-temporal pattern.

15. The method of claim 13, wherein the RNA is encoded by a nucleotide sequence selected from the group consisting of SEQ ID NO:13, SEQ ID NO:62, SEQ ID NO:23, SEQ ID NO:24, and SEQ ID:61.

16. The method of claim 1, wherein said animal cell or said single celled embryo is transformed with said construct by microinjection, transfection or infection, wherein said construct stably integrates into the genome of said cell or said single celled embryo by homologous recombination.

17. A nucleic acid molecule, which encodes a promoter and is transiently activated in a defined spatio-temporal pattern, wherein said nucleic acid molecule comprises the nucleotide sequence of SEQ ID NO:1, SEQ ID NO:8, or SEQ ID NO:60.

18. A nucleic acid molecule, which encodes a promoter having:
a) a nucleotide sequence as shown in SEQ ID NO:1, SEQ ID NO:8 and SEQ ID NO:60;
b) a biologically active fragment of the sequence in a);
c) a nucleic acid molecule which has at least 85% sequence homology to the sequence in a) or b); or
d) a nucleic acid molecule which is capable of hybridizing to the sequence in a) or b) under stringent conditions.

19. A nucleic acid molecule that encodes the coding region of a gene including:
a) a nucleotide sequence selected from the group consisting of SEQ ID NO:63, SEQ ID NO:23, SEQ ID NO:24 and SEQ ID NO 61;
b) a biologically active fragment of any one of the sequences in a);
c) a nucleic acid molecule that has at least 85% sequence homology with any one of the sequences disclosed in a) or b); or
d) a nucleic acid molecule that specifically hybridizes to any one of the sequences disclosed in a) or b) under stringent conditions.

20. A nucleic acid molecule that encodes a blocker molecule that disrupts gametogenesis or embryogenesis in an animal, wherein the blocker molecule is encoded, or partially encoded, by a sequence selected from the group consisting of SEQ ID NO:13, SEQ ID NO:62, SEQ ID NO:23 and SEQ ID NO:61.

21. The nucleic acid molecule of claim 20, wherein the blocker molecule is selected from the group consisting of an antisense RNA, a dsRNA, a sense RNA and a ribozyme.

22. The nucleic acid molecule of claim 21, wherein the molecule is a dsRNA or a sense RNA that when mis-expressed disrupts development in a defined spatio-temporal pattern.

23. A transgenic non-human animal stably transformed with the nucleic acid molecule of claim 17.

24. The transgenic non-human animal of claim 23, wherein the animal is selected from the group consisting of fish, mammals, amphibians, and molluscs.


SEQUENCE LISTING (63 sequences)

SEQ ID NO: 1 - DNA, 1710 bp, Zebrafish
acatagtgttcatcatatataagtacaccctttgaaaatctctcaattcatattttgtgcatatacattagataagtcagtactgaagccaaatctgaagctaatctaagaaaataacatgatagtctaagttagtacactcaaatttatgtaagggaaaatattaggtaaaaaatgtaaaaagatcaaaattcatatatgtatattgtttatatatgtatataggaagctttacaatattatatttgtgcatatacattagactagtcagtattaaagccaaatctggagctaatttaacaaaataacttatgattatagtataaaatttgtacacgcaaatttgtaagttaagcaaatatatatatatatatatatatatatatatatatatatatatatatatatatatatatatatatatatatatatatatatatatatccctcaagatattttttattattgttatttttgttactacagggactagagatgtaaagtcagaattattagcccccttgtatattttcccccccatttctgtttaacggaaagcagatttttttaagcagaccttgaaatggcttttaaaaaattaaaaacttgttattttctagccgaaataaaacaaataagactttctcccttgctctgataaaaatcatttgggaaatattaaaaaaagaacacaatttcaaaggggcactaataattctgacatcaactttaaattttatttatttatcttttggtaactacgacgacaagagatgtaatttagctttatagctatggcacaacatgtcatgttgtagctacattgtcccagaataagtaaataaaagaatattcggctttatacaagtctaaaatagttttacataaaatgttagatcattttaaaacgtttaaagacaacacattgcaataacaaatcaattaaatgaaacctaaaataacgttaacatttacccttcactataaattactatacatgattttaaacagaagatatatccttataaatactgaaaaaatactcaaatacaaatgtagataatttaaattagtgcgcatttaaatttaggatttgtttaaccatacttcagtctcaattgtattgcgtatacattacattctcgttcaaattactaacatgtttacataggataatacataaaatatgccccatgcaggggaaattcggtccatccgcgcgcgcagagtgtgggcatgttcaaacgcttgaatggagagagcgcggcatcattgtgacatcatcagacaacaaaaagccttgcgctcgcgcagcgaagcgctccaatcaatggcacagacgcggcgcgtgctgcacgcagagatgagtctccaaacagccacggaaaacttctgctgaccacaagtttttgatttctttaaaacaaaaacaaaaaatgacaaatccaggattgtgcgatctcgcgctgtcacttttgggattgctgctgtctttgacctgagcgctcgcgcacttcattagagtttagtagagtctagtctgaagtgttgcacaagtatgaacaagaagaggcgacttgagctgcgacgactctctgtcgtgggataaaaaaatcgcttgtggattaaaacacgaattcatgaggaacttagaagacgacgggaacgcagaccggccacagcgcttcctcctccggtaacgcattcaat

SEQ ID NO: 2 - DNA, 1481 bp, Artificial Sequence
  Tetracycline-responsive transcriptional activator protein nucleotide sequence
atgtctagattagataaaagtaaagtgattaacagcgcattagagctgcttaatgaggtcggaatcgaaggtttaacaacccgtaaactcgcccagaagcttggtgtagagcagcctacactgtattggcatgtaaaaaataagcgggctttgctcgacgccttagccattgagatgttagataggcaccatactcacttttgccctttaaaaggggaaagctggcaagattttttacgcaataacgctaaaagttttagatgtgctttactaagtcatcgcaatggagcaaaagtacattcagatacacggcctacagaaaaacagtatgaaactctcgaaaatcaattagcctttttatgccaacaaggtttttcactagagaacgcgttatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtagggggcgcgaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtctggccggaccacgctatctgtgcaaggtccccggacgcgcgctccatgagcagagcgcccgccgccgaggcaagactcgggcggcgccctgcccgtcccaccaggtcaacaggcggtaaccggcctcttcatcgggaatgcgcgcgaccttcagcatcgccggcatgtcccctggcggacgggaagtatcagctcgaccaagctt

SEQ ID NO: 3 - DNA, 447 bp, Artificial Sequence
  Tet responsive element
ctcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctcccggcccc

SEQ ID NO: 4 - DNA, 21 bp, Artificial Sequence
  SMADu1 forward primer
tgcaggtggactttggatccg

SEQ ID NO: 5 - DNA, 21 bp, Artificial Sequence
  SMADL1 reverse primer
gcctaaaggcaacagatgcta

SEQ ID NO: 6 - DNA, 17 bp, Artificial Sequence
  M13 forward primer
gtaaaacgacggccagt

SEQ ID NO: 7 - DNA, 19 bp, Artificial Sequence
  SMAD L2 reverse primer
tagtgctgggctgcaccag

SEQ ID NO: 8 - DNA, 1006 bp, Zebrafish
  misc_feature (478)..(478): n is a, c, g, or t
aagcttacttgtatatttaggttctcctggaccctcgcaattcaacggaaactagtatatcttcatggaatgagttaaacgaaggaatatcttgttttttcttatatatttaggtcattttaatcaccctttgccttaatgtttggccagaggagaaatggttgtgcccaactgagcctggtttctctctcttttatctattggtaaagttttgtttctctacgctggcttgcttggttttggtacttgtggagttgtgcatcgatggatttgctcttcagtgtttggacttttagttgtgaaatttaaaccacactgaactaaactgaacttcaactctaaaaactggactgacacagtttcagtttactagaacttttatgttaagctgctttaacacaatctacattgtaaaagcgctgtagaaataaacataaattgaattaaattcatttgttaatttaaggaaatttggtgnaatttcagggttaatattttaattngcactcacagaatttttaaaaatgaattaaaatattggaaaatctattcaactccctgaatttgctttcataattaatagattatgcatgttttatttccaaactgaaatcaatttctctctttttttttttttatctgcaggtggactttgagtccggtgtcagtctctgaccacaaccaatatctggcatggattagtttataaaatctcctaactgcctggttgtgtgtttccagccttgattcctcaattgccctttacgctaattctcgcagtagttgtgacccagttcctcccccggcttcactgcaggccttcctgagccccaagtaccagcagctgcgtcctgctttccacttcctgtccttggtcctgcaaggctaagcctgtccacttcccccctccccccctgacatacacaaacacacacataatcatcttcctggcacactgctggccgaggacgctccagatttggcttcctggtgcagcccagcacta

SEQ ID NO: 9 - DNA, 26 bp, Artificial Sequence
  zfEx 1-3.EcoF Forward Primer
accccgaattcatgaggaacttagga

SEQ ID NO: 10 - DNA, 29 bp, Artificial Sequence
  zfEx1-3.SalR Reverse Primer
atcagctcgtcgacaggaatggaggtaag

SEQ ID NO: 11 - DNA, 31 bp, Artificial Sequence
  Bex1i.PstF 2 Forward Primer
acacctgcagatgaggaacttaggagacgac

SEQ ID NO: 12 - DNA, 26 bp, Artificial Sequence
  Bex1i.SalR Reverse Primer
tactgagggtcgactgccgatttgct

SEQ ID NO: 13 - DNA, 1126 bp, Artificial Sequence
  Blocker Molecule
gaattcatgaggaacttaggagacgacgggaacgcagaccggccacagcgcttcctcctccggaactgactgatcatggtcgccgtggtccgcgctctcacggtgctgttgctcggtcaggtgttgctgggaggtgccgttggactcattcccgagatcgaccgacggaaatacagtgattcggggagacacacaccggagcgaactgatacaaacttcctgaacgagtttgagctacgcttgctcaatatgttcggattgaagcgaaaacccaccccaagcaaatcggcagtggtccctcagtacatgctggacttgtattatatgcactctgaaaacgatgacccgaacattcggcgcccgaggagcactatgggaaaacatgtagaaagggcagccagcagagcaaacacgatacgaagttttcatcacgaagaggctttcgaggcactgtccagcctgaaaggaaaaacaacgcagcagtttttcttcaaccttacctccattcctgtcgactgccgatttgcttggggtgggttttcgcttcaatccgaacatattgagcaagcgtagctcaaactcgttcaggaagtttgtatcagttcgctccggtgtgtgtctccccgaatcactgtatttccgtcggtcgatctcgggaatgagtccaacggcacctcccagcaacacctgaccgagcaacagcaccgtgagagcgcggaccacggcgaccatgatcagtcagttccggaggaggaagcgctgtggccggtctgcgttcccgtcgtctcctaagttcctcatctgcagcaattggatatcaagcttatcgatgatgatccagacatgataagatacattgatgagtttggacaaaccncanctagaatgcagtgaaaaaaatgctttatttgtgaaatttgtgatgctattgctttatttgtaaccattataagctgcaataaacaagttaacnacaacaattgcattcattttatgtttcaggttcagngggaggtgtgggaggttttttaaagcaagtaaaacctctncaaatgtggtatggctgattatgatcctctagatcagatccactagttctagagcggccgccaccgcggtggagctc

SEQ ID NO: 14 - DNA, 8282 bp, Artificial Sequence
  pBIT(Bmp2)-bmp2ds construct refereed to as pSF1
actagttctagagcggccgcctgcaggaattcggggccgcggaggctggatcggtcccggtgtcttctatggaggtcaaaacagcgtggatggcgtctccaggcgatctgacggttcactaaacgagctctgcttatataggtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcggtacccggggatcctctagtcagaattcatgaggaacttaggagacgacgggaacgcagaccggccacagcgcttcctcctccggaactgactgatcatggtcgccgtggtccgcgctctcacggtgctgttgctcggtcaggtgttgctgggaggtgccgttggactcattcccgagatcgaccgacggaaatacagtgattcggggagacacacaccggagcgaactgatacaaacttcctgaacgagtttgagctacgcttgctcaatatgttcggattgaagcgaaaacccaccccaagcaaatcggcagtggtccctcagtacatgctggacttgtattatatgcactctgaaaacgatgacccgaacattcggcgcccgaggagcactatgggaaaacatgtagaaagggcagccagcagagcaaacacgatacgaagttttcatcacgaagaggctttcgaggcactgtccagcctgaaaggaaaaacaacgcagcagtttttcttcaaccttacctccattcctgtcgactgccgatttgcttggggtgggttttcgcttcaatccgaacatattgagcaagcgtagctcaaactcgttcaggaagtttgtatcagttcgctccggtgtgtgtctccccgaatcactgtatttccgtcggtcgatctcgggaatgagtccaacggcacctcccagcaacacctgaccgagcaacagcaccgtgagagcgcggaccacggcgaccatgatcagtcagttccggaggaggaagcgctgtggccggtctgcgttcccgtcgtctcctaagttcctcatctgcagcaattggatatcaagctctgacgcgtgctagcgcggcctcgacgatatctctagactgagaacttcagggtgagtttggggacccttgattgttctttctttttcgctattgaaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaacttttttcgttaaactttagcttgcatttgtaacgaatttttaaattcactttcgtttatttgtcagattgtaagtactttctctaatcacttttttttcaaggcaatcagggtaattatattgtacttcagcacagttttagagaacaattgttataattaaatgataaggtagaatatttctgcatataaattctggctggcgtggaaatattcttattggtagaaacaactacatcctggtaatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaattcactcctcaggtgcaggctgcctatcagaaggtggtggctggtgtggccaatgccctggctcacaaataccactgagatctttttccctctgccaaaaattatggggacatcatgaagccccttgagcatctgacttctgggtaataaaggaaatttattttcattgcaatagtgtgtgggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaactcgactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgcgacaagctttacaatattatatttgtgcatatacattagactagtcagtattaaagccaaatctggagctaatttaacaaaataacttatgattatagtataaaatttgtacacgcaaatttgtaagttaagcaaatatatatatatatatatatatatatatatatatatatatatatatatatatatatatatatatatatatatatatatatatatatccctcaagatattttttattattgttatttttgttactacagggactagagatgtaaagtcagaattattagcccccttgtatattttcccccccatttctgtttaacggaaagcagatttttttaagcagaccttgaaatggcttttaaaaaattaaaaacttgttattttctagccgaaataaaacaaataagactttctcccttgctctgataaaaatcatttgggaaatattaaaaaaagaacacaatttcaaaggggcactaataattctgacatcaactttaaattttatttatttatcttttggtaactacgacgacaagagatgtaatttagctttatagctatggcacaacatgtcatgttgtagctacattgtcccagaataagtaaataaaagaatattcggctttatacaagtctaaaatagttttacataaaatgttagatcattttaaaacgtttaaagacaacacattgcaataacaaatcaattaaatgaaacctaaaataacgttaacatttacccttcactataaattactatacatgattttaaacagaagatatatccttataaatactgaaaaaatactcaaatacaaatgtagataatttaaattagtgcgcatttaaatttaggatttgtttaaccatacttcagtctcaattgtattgcgtatacattacattctcgttcaaattactaacatgtttacataggataatacataaaatatgccccatgcaggggaaattcggtccatccgcgcgcgcagagtgtgggcatgttcaaacgcttgaatggagagagcgcggcatcattgtgacatcatcagacaacaaaaagccttgcgctcgcgcagcgaagcgctccaatcaatggcacagacgcggcgcgtgctgcacgcagagatgagtctccaaacagccacggaaaacttctgctgaccacaagtttttgatttctttaaaacaaaaacaaaaaatgacaaatccaggattgtgcgatctcgcgctgtcacttttgggattgctgctgtctttgacctgagcgctcgcgcacttcattagagtttagtagagtctagtctgaagtgttgcacaagtatgaacaagaagaggcgacttgagctgcgacgactctctgtcgtgggataaaaaaatcgcttgtggattaaaacacgaattcatatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtagggggcgcgaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtctggccggaccacgctatctgtgcaaggtccccggacgcgcgctccatgagcagagcgcccgccgccgaggcaagactcgggcggcgccctgcccgtcccaccaggtcaacaggcggtaaccggcctcttcatcgggaatgcgcgcgaccttcagcatcgccggcatgtcccctggcggacgggaagtatcagctcgaccaagcttgatatcgaattcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatccgcggggatcc

SEQ ID NO: 15 - DNA, 7713 bp, Artificial Sequence
  pBIT(CMV)-bmp2ds referred to as pSF2
ctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccccgaattcatatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtagggggcgcgaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtctggccggaccacgctatctgtgcaaggtccccggacgcgcgctccatgagcagagcgcccgccgccgaggcaagactcgggcggcgccctgcccgtcccaccaggtcaacaggcggtaaccggcctcttcatcgggaatgcgcgcgaccttcagcatcgccggcatgtcccctggcggacgggaagtatcagctcgaccaagcttgatatcgaattcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatccgcggggatccactagttctagagcggccgcctgcaggaattcggggccgcggaggctggatcggtcccggtgtcttctatggaggtcaaaacagcgtggatggcgtctccaggcgatctgacggttcactaaacgagctctgcttatataggtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcggtacccggggatcctctagtcagaattcatgaggaacttaggagacgacgggaacgcagaccggccacagcgcttcctcctccggaactgactgatcatggtcgccgtggtccgcgctctcacggtgctgttgctcggtcaggtgttgctgggaggtgccgttggactcattcccgagatcgaccgacggaaatacagtgattcggggagacacacaccggagcgaactgatacaaacttcctgaacgagtttgagctacgcttgctcaatatgttcggattgaagcgaaaacccaccccaagcaaatcggcagtggtccctcagtacatgctggacttgtattatatgcactctgaaaacgatgacccgaacattcggcgcccgaggagcactatgggaaaacatgtagaaagggcagccagcagagcaaacacgatacgaagttttcatcacgaagaggctttcgaggcactgtccagcctgaaaggaaaaacaacgcagcagtttttcttcaaccttacctccattcctgtcgactgccgatttgcttggggtgggttttcgcttcaatccgaacatattgagcaagcgtagctcaaactcgttcaggaagtttgtatcagttcgctccggtgtgtgtctccccgaatcactgtatttccgtcggtcgatctcgggaatgagtccaacggcacctcccagcaacacctgaccgagcaacagcaccgtgagagcgcggaccacggcgaccatgatcagtcagttccggaggaggaagcgctgtggccggtctgcgttcccgtcgtctcctaagttcctcatctgcagcaattggatatcaagctctgacgcgtgctagcgcggcctcgacgatatctctagactgagaacttcagggtgagtttggggacccttgattgttctttctttttcgctattgaaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaacttttttcgttaaactttagcttgcatttgtaacgaatttttaaattcactttcgtttatttgtcagattgtaagtactttctctaatcacttttttttcaaggcaatcagggtaattatattgtacttcagcacagttttagagaacaattgttataattaaatgataaggtagaatatttctgcatataaattctggctggcgtggaaatattcttattggtagaaacaactacatcctggtaatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaattcactcctcaggtgcaggctgcctatcagaaggtggtggctggtgtggccaatgccctggctcacaaataccactgagatctttttccctctgccaaaaattatggggacatcatgaagccccttgagcatctgacttctgggtaataaaggaaatttattttcattgcaatagtgtgtgggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttca

SEQ ID NO: 16 - DNA, 7998 bp, Artificial Sequence
  pBIT(smad2)-BMPp2ds construct refereed to as pSF3
ctcgaggagcttggcccattgcatacgttgtatccatatcataatatgtacatttatattggctcatgtccaacattaccgccatgttgacattgattattgactagtatatcttcatggaatgagttaaacgaaggaatatcttgttttttcttatatatttaggtcattttaatcaccctttgccttaatgtttggccagaggagaaatggttgtgcccaactgagcctggtttctctctcttttatctattggtaaagttttgtttctctacgctggcttgcttggttttggtacttgtggagttgtgcatcgatggatttgctcttcagtgtttggacttttagttgtgaaatttaaaccacactgaactaaactgaacttcaactctaaaaactggactgacacagtttcagtttactagaacttttatgttaagctgctttaacacaatctacattgtaaaagcgctgtagaaataaacataaattgaattaaattcatttgttaatttaaggaaatttggtgnaatttcagggttaatattttaattngcactcacagaatttttaaaaatgaattaaaatattggaaaatctattcaactccctgaatttgctttcataattaatagattatgcatgttttatttccaaactgaaatcaatttctctctttttttttttttatctgcaggtggactttgagtccggtgtcagtctctgaccacaaccaatatctggcatggattagtttataaaatctcctaactgcctggttgtgtgtttccagccttgattcctcaattgccctttacgctaattctcgcagtagttgtgacccagttcctcccccggcttcactgcaggccttcctgagccccaagtaccagcagctgcgtcctgctttccacttcctgtccttggtcctgcaaggctaagcctgtccacttcccccctccccccctgacatacacaaacacacacataatcatcttcctggcacactgctggccgaggacgctccagatttggcttcctggtgcagcccagcactaatcactagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtagggggcgcgaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtctggccggaccacgctatctgtgcaaggtccccggacgcgcgctccatgagcagagcgcccgccgccgaggcaagactcgggcggcgccctgcccgtcccaccaggtcaacaggcggtaaccggcctcttcatcgggaatgcgcgcgaccttcagcatcgccggcatgtcccctggcggacgggaagtatcagctcgaccaagcttgatatcgaattcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatccgcggggatccactagttctagagcggccgcctgcaggaattcggggccgcggaggctggatcggtcccggtgtcttctatggaggtcaaaacagcgtggatggcgtctccaggcgatctgacggttcactaaacgagctctgcttatataggtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcggtacccggggatcctctagtcagaattcatgaggaacttaggagacgacgggaacgcagaccggccacagcgcttcctcctccggaactgactgatcatggtcgccgtggtccgcgctctcacggtgctgttgctcggtcaggtgttgctgggaggtgccgttggactcattcccgagatcgaccgacggaaatacagtgattcggggagacacacaccggagcgaactgatacaaacttcctgaacgagtttgagctacgcttgctcaatatgttcggattgaagcgaaaacccaccccaagcaaatcggcagtggtccctcagtacatgctggacttgtattatatgcactctgaaaacgatgacccgaacattcggcgcccgaggagcactatgggaaaacatgtagaaagggcagccagcagagcaaacacgatacgaagttttcatcacgaagaggctttcgaggcactgtccagcctgaaaggaaaaacaacgcagcagtttttcttcaaccttacctccattcctgtcgactgccgatttgcttggggtgggttttcgcttcaatccgaacatattgagcaagcgtagctcaaactcgttcaggaagtttgtatcagttcgctccggtgtgtgtctccccgaatcactgtatttccgtcggtcgatctcgggaatgagtccaacggcacctcccagcaacacctgaccgagcaacagcaccgtgagagcgcggaccacggcgaccatgatcagtcagttccggaggaggaagcgctgtggccggtctgcgttcccgtcgtctcctaagttcctcatctgcagcaattggatatcaagctctgacgcgtgctagcgcggcctcgacgatatctctagactgagaacttcagggtgagtttggggacccttgattgttctttctttttcgctattgaaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaacttttttcgttaaactttagcttgcatttgtaacgaatttttaaattcactttcgtttatttgtcagattgtaagtactttctctaatcacttttttttcaaggcaatcagggtaattatattgtacttcagcacagttttagagaacaattgttataattaaatgataaggtagaatatttctgcatataaattctggctggcgtggaaatattcttattggtagaaacaactacatcctggtaatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaattcactcctcaggtgcaggctgcctatcagaaggtggtggctggtgtggccaatgccctggctcacaaataccactgagatctttttccctctgccaaaaattatggggacatcatgaagccccttgagcatctgacttctgggtaataaaggaaatttattttcattgcaatagtgtgtgggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttca

SEQ ID NO: 17 - DNA, 8611 bp, Artificial Sequence
  pBITsmad2)-BMP2sense construct refereed to as pSF4
ctcgaggagcttggcccattgcatacgttgtatccatatcataatatgtacatttatattggctcatgtccaacattaccgccatgttgacattgattattgactagtatatcttcatggaatgagttaaacgaaggaatatcttgttttttcttatatatttaggtcattttaatcaccctttgccttaatgtttggccagaggagaaatggttgtgcccaactgagcctggtttctctctcttttatctattggtaaagttttgtttctctacgctggcttgcttggttttggtacttgtggagttgtgcatcgatggatttgctcttcagtgtttggacttttagttgtgaaatttaaaccacactgaactaaactgaacttcaactctaaaaactggactgacacagtttcagtttactagaacttttatgttaagctgctttaacacaatctacattgtaaaagcgctgtagaaataaacataaattgaattaaattcatttgttaatttaaggaaatttggtgnaatttcagggttaatattttaattngcactcacagaatttttaaaaatgaattaaaatattggaaaatctattcaactccctgaatttgctttcataattaatagattatgcatgttttatttccaaactgaaatcaatttctctctttttttttttttatctgcaggtggactttgagtccggtgtcagtctctgaccacaaccaatatctggcatggattagtttataaaatctcctaactgcctggttgtgtgtttccagccttgattcctcaattgccctttacgctaattctcgcagtagttgtgacccagttcctcccccggcttcactgcaggccttcctgagccccaagtaccagcagctgcgtcctgctttccacttcctgtccttggtcctgcaaggctaagcctgtccacttcccccctccccccctgacatacacaaacacacacataatcatcttcctggcacactgctggccgaggacgctccagatttggcttcctggtgcagcccagcactaatcactagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtagggggcgcgaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtctggccggaccacgctatctgtgcaaggtccccggacgcgcgctccatgagcagagcgcccgccgccgaggcaagactcgggcggcgccctgcccgtcccaccaggtcaacaggcggtaaccggcctcttcatcgggaatgcgcgcgaccttcagcatcgccggcatgtcccctggcggacgggaagtatcagctcgaccaagcttgatatcgaattcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatccgcggggatccactagttctagagcggccgcctgcaggaattcggggccgcggaggctggatcggtcccggtgtcttctatggaggtcaaaacagcgtggatggcgtctccaggcgatctgacggttcactaaacgagctctgcttatataggtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcggtacccggggatcctctagtcagaattcatgaggaacttaggagacgacgggaacgcagaccggccacagcgcttcctcctccggaactgactgatcatggtcgccgtggtccgcgctctcacggtgctgttgctcggtcaggtgttgctgggaggtgccgttggactcattcccgagatcgaccgacggaaatacagtgattcggggagacacacaccggagcgaactgatacaaacttcctgaacgagtttgagctacgcttgctcaatatgttcggattgaagcgaaaacccaccccaagcaaatcggcagtggtccctcagtacatgctggacttgtattatatgcactctgaaaacgatgacccgaacattcggcgcccgaggagcactatgggaaaacatgtagaaagggcagccagcagagcaaacacgatacgaagttttcatcacgaagaggctttcgaggcactgtccagcctgaaaggaaaaacaacgcagcagtttttcttcaaccttacctccattcctggcgaggagctgatctccgctgcggagctgcgcattttcagggaccaagttctcggagatgccagtacgagtggcttccacagaattaacatttacgaggtgttcaggccagctttggccccctccaaagagcctctaaccagacttctggacacccgtctggtgcaggactctcacacgcgctgggaaagcttcgacgtgggttcagctgtggcacgctgggcccgcgaatcccagcacaaccacgggctccttgtagaggtgctccatcctaaggagtcagaagtatccgaggaggctgagagcaaccggaggaagcacgtgagggtcagtcgttcccttcacgcggatgaggactcgtgggcacaagcccgacctctgctggtaacctacagccatgacggtcaaggcacagccgtcttgcattcgaaccgagaaaagcggcaggctcgacgagggcaaaagccgaggagaaagcaccaccagcgctcgaactgtaggcgacatgctctctatgtggacttcagtgatgtcggctggaacgagtggatcgtggcaccgccaggctatcatgctttctactgccatggcgagtgtccgttccctctgccggaccatctaaactccaccaaccatgccattgtccagacgctggtgaactcggtcaactccaacattcccaaagcctgttgcatcccgacggagctcagccctatctcactgctgtacctggacgagtacgagaaggtcattcttaaaaactaccaggacatggtggtggagggctgtgggtgccgatgagaacaatctccccaatgaagacttttatttatacaaaagagcgagctatttggaggaagaaaagaaatatatatgaatatatttatgttgaatgaacaaaacaaaaaaaaaaaaaaaaaaactcgactgacgcgtgctagcgcggcctcgacgatatctctagactgagaacttcagggtgagtttggggacccttgattgttctttctttttcgctattgaaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaacttttttcgttaaactttagcttgcatttgtaacgaatttttaaattcactttcgtttatttgtcagattgtaagtactttctctaatcacttttttttcaaggcaatcagggtaattatattgtacttcagcacagttttagagaacaattgttataattaaatgataaggtagaatatttctgcatataaattctggctggcgtggaaatattcttattggtagaaacaactacatcctggtaatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaattcactcctcaggtgcaggctgcctatcagaaggtggtggctggtgtggccaatgccctggctcacaaataccactgagatctttttccctctgccaaaaattatggggacatcatgaagccccttgagcatctgacttctgggtaataaaggaaatttattttcattgcaatagtgtgtgggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttca

SEQ ID NO: 18 - DNA, 6890 bp, Artificial Sequence
  pBIT(CMV2)-EGFP
ctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccccgaattcatatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtagggggcgcgaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtctggccggaccacgctatctgtgcaaggtccccggacgcgcgctccatgagcagagcgcccgccgccgaggcaagactcgggcggcgccctgcccgtcccaccaggtcaacaggcggtaaccggcctcttcatcgggaatgcgcgcgaccttcagcatcgccggcatgtcccctggcggacgggaagtatcagctcgaccaagcttgatatcgaattcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatccgcggggatccactagttctagagcggccgcctgcaggaattcggggccgcggaggctggatcggtcccggtgtcttctatggaggtcaaaacagcgtggatggcgtctccaggcgatctgacggttcactaaacgagctctgcttatataggtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcggtacccggggatcctctagtcagctgacgcgtgctagcgcggcctcgacgatatctctagactgagaacttcagggtgagtttggggacccttgattgttctttctttttcgctattgaaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaacttttttcgttaaactttagcttgcatttgtaacgaatttttaaattcactttcgtttatttgtcagattgtaagtactttctctaatcacttttttttcaaggcaatcagggtaattatattgtacttcagcacagttttagagaacaattgttataattaaatgataaggtagaatatttctgcatataaattctggctggcgtggaaatattcttattggtagaaacaactacatcctggtaatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaattcactcctcaggtgcaggctgcctatcagaaggtggtggctggtgtggccaatgccctggctcacaaataccactgagatctttttccctctgccaaaaattatggggacatcatgaagccccttgagcatctgacttctgggtaataaaggaaatttattttcattgcaatagtgtgtgggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttca

SEQ ID NO: 19 - DNA, 6624 bp, Artificial Sequence
  pBITdHSP2)-EGFP
ctcgagcggccgccagtgtgatggatatctgcagaattcgccctttgtaaaacgacggccagtgaattgtaatacgactcactatagggcgaattggccgttattcgttattctctcttttctttttgggtctctccctctctgcactaatgctctctcactctgtcacacagtaaacggcatactgctctcgttggttcgagagagcgcgcctcgaatgttcgcgaaaagagcgccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaagagaactctgaatagggaattgggaattcgttaacagatctgcggccgcggtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtagggggcgcgaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtctggccggaccacgctatctgtgcaaggtccccggacgcgcgctccatgagcagagcgcccgccgccgaggcaagactcgggcggcgccctgcccgtcccaccaggtcaacaggcggtaaccggcctcttcatcgggaatgcgcgcgaccttcagcatcgccggcatgtcccctggcggacgggaagtatcagctcgaccaagcttgatatcgaattcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatccgcggggatccactagttctagagcggccgcctgcaggaattcggggccgcggaggctggatcggtcccggtgtcttctatggaggtcaaaacagcgtggatggcgtctccaggcgatctgacggttcactaaacgagctctgcttatataggtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcggtacccggggatcctctagtcagctgacgcgtgctagcgcggcctcgacgatatctctagactgagaacttcagggtgagtttggggacccttgattgttctttctttttcgctattgaaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaacttttttcgttaaactttagcttgcatttgtaacgaatttttaaattcactttcgtttatttgtcagattgtaagtactttctctaatcacttttttttcaaggcaatcagggtaattatattgtacttcagcacagttttagagaacaattgttataattaaatgataaggtagaatatttctgcatataaattctggctggcgtggaaatattcttattggtagaaacaactacatcctggtaatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaattcactcctcaggtgcaggctgcctatcagaaggtggtggctggtgtggccaatgccctggctcacaaataccactgagatctttttccctctgccaaaaattatggggacatcatgaagccccttgagcatctgacttctgggtaataaaggaaatttattttcattgcaatagtgtgtgggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaactcgactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttca

SEQ ID NO: 20 - DNA, 7910 bp, Artificial Sequence
  pBIT(dHSP)-RFP-oHoxDS/BH
ctcgagcggccgccagtgtgatggatatctgcagaattcgccctttgtaaaacgacggccagtgaattgtaatacgactcactatagggcgaattggccgttattcgttattctctcttttctttttgggtctctccctctctgcactaatgctctctcactctgtcacacagtaaacggcatactgctctcgttggttcgagagagcgcgcctcgaatgttcgcgaaaagagcgccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaagagaactctgaatagggaattgggaattcgttaacagatctgcggccgcggtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtagggggcgcgaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtctggccggaccacgctatctgtgcaaggtccccggacgcgcgctccatgagcagagcgcccgccgccgaggcaagactcgggcggcgccctgcccgtcccaccaggtcaacaggcggtaaccggcctcttcatcgggaatgcgcgcgaccttcagcatcgccggcatgtcccctggcggacgggaagtatcagctcgaccaagcttgcatgcctgcaggtcgactctagagtcgcggccgctacaggaacaggtggtggcggccctcggtgcgctcgtactgctccacgatggtgtagtcctcgttgtgggaggtgatgtccagcttggagtccacgtagtagtagccgggcagctgcacgggcttcttggccatgtagatggacttgaactccaccaggtagtggccgccgtccttcagcttcagggccttgtggatctcgcccttcagcacgccgtcgcgggggtacaggcgctcggtggaggcctcccagcccatggtcttcttctgcattacggggccgtcggaggggaagttcacgccgatgaacttcaccttgtagatgaagcagccgtcctgcagggaggagtcctgggtcacggtcaccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactccttgatgacgttcttggaggagcgcaccatggtggcgaccggtggatcccgggccgcctgcaggaattcggggccgcggaggctggatcggtcccggtgtcttctatggaggtcaaaacagcgtggatggcgtctccaggcgatctgacggttcactaaacgagctctgcttatataggtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcggtacccggggatcctctagtcagctgacgcgttgggctagtgattggatctgaccgtccatcgcaataaaatgagccattatgagatcgaaagggtctacgaaaatccgttcggaaaaaatcagaaaatcatcaaagccgaatataattaaaatgtattactagctaaagaaatcatcactaatatagaatgtagaatgaacccatgtatattagatactaattgtatcgtaagactttcaaaagtctacaagacattaaatgacaagttgactttaaatttcaaataaataatttattttttctataagcaataacatttttgctaaattaagacttggtaattaggtaatactattgttgttctatggaatattcgatcgaaacattcttatcagtctcaaaaacttaagacaaacttataatataacccatatgttataacccattgatgaacaaaaattagactctttggccttagtcgacggatccccgacaccagaccaactggtaatggtagcgaccggcgctcagctggaattaggccttctagtgaatcatccgtcggttttggaaccagatcttcacttgcctttcggagagagcgagattttgggccagctcggctttcctcctgatggtgatgtagcgactgtagaggaactccttttccagctccagcgtctgagtcgacaggaatggaggtaaggttgaagaaaaactgctgcgttgtttttcctttcaggctggacagtgcctcgaaagcctcttcgtgatgaaaacttcgtatcgtgtttgctctgctggctgccctttctacatgttttcccatagtgctcctcgggcgccgaatgttcgggtcatcgttttcagagtgcatataatacaagtccagcatgtactgagggaccactgccgatttgcttggggtgggttttcgcttcaatccgaacatattgagcaagcgtagctcaaactcgttcaggaagtttgtatcagttcgctccggtgtgtgtctccccgaatcactgtatttccgtcggtcgatctcgggaatgagtccaacggcacctcccagcaacacctgaccgagcaacagcaccgtgagagcgcggaccacggcgaccatgatcagtcagttccggaggaggaagcgctgtggccggtctgcgttcccgtcgtctcctaagttcctcatgaattcgattcagacgctggagctggaaaaggagttcctctacagtcgctacatcaccatcaggaggaaagccgagctggcccaaaatctcgctctctccgaaaggcaagtgaagatctggttccaaaaccgacggatgaatcactagcgcggcctcgacgatatctctagactgagaacttcagggtgagtttggggacccttgattgttctttctttttcgctattgaaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaacttttttcgttaaactttagcttgcatttgtaacgaatttttaaattcactttcgtttatttgtcagattgtaagtactttctctaatcacttttttttcaaggcaatcagggtaattatattgtacttcagcacagttttagagaacaattgttataattaaatgataaggtagaatatttctgcatataaattctggctggcgtggaaatattcttattggtagaaacaactacatcctggtaatcatcctgcctttctctttatggttacaatgatatacactgtttgagatgaggataaaatactctgagtccaaaccgggcccctctgctaaccatgttcatgccttcttctttttcctacagctcctgggcaacgtgctggttgttgtgctgtctcatcattttggcaaagaattcactcctcaggtgcaggctgcctatcagaaggtggtggctggtgtggccaatgccctggctcacaaataccactgagatctttttccctctgccaaaaattatggggacatcatgaagccccttgagcatctgacttctgggtaataaaggaaatttattttcattgcaatagtgtgtgggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaactcgactgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttca

SEQ ID NO: 21 - DNA, 5919 bp, Artificial Sequence
  pHSP-GUS Plasmid
gggcgaattgggcccgacgtcgcatgctcccggccgccatggcggccgcgggaattcgattccaggatcctgaccgtccatcgcaataaaatgagccattatgagatcgaaagggtctacgaaaatccgttcggaaaaaatcagaaaatcatcaaagccgaatataattaaaatgtattactagctaaagaaatcatcactaatatagaatgtagaatgaaccatgtatatagatactaatgtatcgtaagactttcaaaagtctacaagacattaaatgacaagttgactttaaatttcaaataaataatttattttttctataagcaataacatttttgctaaattaagacttggtaattaggtaatactattgttgttctatggaatattcgatcgaaacattcttatcagtctcaaaaacttaaaacaaacttataatataacccatatgttataacccattgatgaacaaaaattagactctttggccttagtcgacggatccccgacaccagaccaactggtaatggtagcgaccggcgctcagctggaattaggccttctagaccgcggccgcagatctcgacgtaggcctttgaattccccaccgaggctgtagccgacgatggtgcgccaggagagttgttgattcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgcgaaatattcccgtgcaccttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgtaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcgatccagactgaatgcccacaggccgtcgagttttttgatttcacgggttggggtttctacaggacggaccatgggacgtcgagatctgttaacgaattcccaattccctattcagagttctcttcttgtattcaataattacttcttggcagatttcagtagttgcagttgatttacttggttgctggttacttttaattgattcactttaacttgcactttactgcagattgtttagcttgttcagctgcgcttgtttatttgcttagctttcgcttagcgacgtgttcactttgcttgtttgaattgaattgtcgctccgtagacgaagcgctctatttatactccggcgctcttttcgcgaacattcgaggcgcgctctctcgaaccaacgagagcagtatgccgtttactgtgtgacagagtgagagagcattagtgcagagagggagacccaaaaagaaaagagagaataacgaataacggccagagaaatttctcgagttttcttctgccaaacaaatgacctaccacaataaccagtttgttttgggattctaggaattctatcactagtgaattcgcggccgcctgcaggtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggcgaaattgtaaacgttaatattttgttaaaattcgcgttaaatatttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 22 - DNA, 3968 bp, Artificial Sequence
  pHSP70-1MCS Plasmid
tgtaaaacgacggccagtgaattgtaatacgactcactatagggcgaattgggccctctagatgcatgctcgagcggccgccagtgtgatggatatctgcagaattcgccctttgtaaaacgacggccagtgaattgtaatacgactcactatagggcgaattggccgttattcgttattctctcttttctttttgggtctctccctctctgcactaatgctctctcactctgtcacacagtaaacggcatactgctctcgttggttcgagagagcgcgcctcgaatgttcgcgaaaagagcgccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaagagaactctgaatagggaattgggaattcgttaacagatctgcggccgcggtctagaaggcctaattccagctgagcgccggtcgctaccattaccagttggtctggtgtcggggatccgtcgactaaggccaaagagtctaatttttgttcatcaatgggttataacatatgggttatattataagtttgtcttaagtttttgagactgataagaatgtttcgatcgaatattccatagaacaacaatagtattacctaattaccaagtcttaatttagcaaaaatgttattgcttatagaaaaaataaattatttatttgaaatttaaagtcaacttgtcatttaatgtcttgtagacttttgaaagtcttacgatacaattagtatctaatatacatgggttcattctacattctatattagtgatgatttctttagctagtaatacattttaattatattcggctttgatgattttctgattttttccgaacggattttcgtagaccctttcgatctcataatggctcattttattgcgatggacggtcagatccaatcactagcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggcgaaattgtaaacgttaatattttgttaaaattcgcgttaaatatttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgt

SEQ ID NO: 23 - DNA, 129 bp, Pacific Oyster
cagacgctggagctggaaaaggagttcctctacagtcgctacatcaccatcaggaggaaagccgagctggcccaaaatctcgctctctccgaaaggcaagtgaagatctggttccaaaaccgacggatg

SEQ ID NO: 24 - DNA, 129 bp, Pacific Oyster
cagacgttggagttggaaaaggagtttcacagcaaaaagtacttatcgctgactgaacgatctcatattgcacataatctaaaattaagtgaagtccaagtaaaaatttggttccaaaaccggcgcatg

SEQ ID NO: 25 - DNA, 18 bp, Artificial Sequence
  Oyster specific antisense
gagatcgttcagtcagcg

SEQ ID NO: 26 - DNA, 17 bp, Artificial Sequence
  Broader spectrum antisense
catgsgssggttttgga

SEQ ID NO: 27 - DNA, 25 bp, Artificial Sequence
  CG1.1.Sal.for Forward Primer
atggatgtcgactcagacgctggag

SEQ ID NO: 28 - DNA, 25 bp, Artificial Sequence
  CG1.1.Pst.rev Reverse Primer
gattcactagtcaattcctgcagtt

SEQ ID NO: 29 - DNA, 4626 bp, Artificial Sequence
  pHSP-oHoxDS/BH Plasmid
tgtaaaacgacggccagtgaattgtaatacgactcactatagggcgaattggccgttattcgttattctctcttttctttttgggtctctccctctctgcactaatgctctctcactctgtcacacagtaaacggcatactgctctcgttggttcgagagagcgcgcctcgaatgttcgcgaaaagagcgccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaagagaactctgaatagggaattgggaattcactagtgattcatccgtcggttttggaaccagatcttcacttgcctttcggagagagcgagattttgggccagctcggctttcctcctgatggtgatgtagcgactgtagaggaactccttttccagctccagcgtctgaatcgaattcatgaggaacttaggagacgacgggaacgcagaccggccacagcgcttcctcctccggaactgactgatcatggtcgccgtggtccgcgctctcacggtgctgttgctcggtcaggtgttgctgggaggtgccgttggactcattcccgagatcgaccgacggaaatacagtgattcggggagacacacaccggagcgaactgatacaaacttcctgaacgagtttgagctacgcttgctcaatatgttcggattgaagcgaaaacccaccccaagcaaatcggcagtggtccctcagtacatgctggacttgtattatatgcactctgaaaacgatgacccgaacattcggcgcccgaggagcactatgggaaaacatgtagaaagggcagccagcagagcaaacacgatacgaagttttcatcacgaagaggctttcgaggcactgtccagcctgaaaggaaaaacaacgcagcagtttttcttcaaccttacctccattcctgtcgactcagacgctggagctggaaaaggagttcctctacagtcgctacatcaccatcaggaggaaagccgagctggcccaaaatctcgctctctccgaaaggcaagtgaagatctggttccaaaaccgacggatgattcactagaaggcctaattccagctgagcgccggtcgctaccattaccagttggtctggtgtcggggatccgtcgactaaggccaaagagtctaatttttgttcatcaatgggttataacatatgggttatattataagtttgtcttaagtttttgagactgataagaatgtttcgatcgaatattccatagaacaacaatagtattacctaattaccaagtcttaatttagcaaaaatgttattgcttatagaaaaaataaattatttatttgaaatttaaagtcaacttgtcatttaatgtcttgtagacttttgaaagtcttacgatacaattagtatctaatatacatgggttcattctacattctatattagtgatgatttctttagctagtaatacattttaattatattcggctttgatgattttctgattttttccgaacggattttcgtagaccctttcgatctcataatggctcattttattgcgatggacggtcagatccaatcactagcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggcgaaattgtaaacgttaatattttgttaaaattcgcgttaaatatttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgt

SEQ ID NO: 30 - DNA, 7713 bp, Artificial Sequence; identical to SEQ ID NO: 15
  pBIT(CMV)-EGFP-zfBMP(DS) expression vector

SEQ ID NO: 31 - DNA, 28 bp, Artificial Sequence
  Dmhsp Forward Primer
gaattcctagaatcccaaaacaaactgg

SEQ ID NO: 32 - DNA, 33 bp, Artificial Sequence
  Dmhst Reverse Primer
ggatcctgaccgtccatcgcaataaaatgagcc

SEQ ID NO: 33 - DNA, 24 bp, Artificial Sequence
  Goosecoid Reverse  Primer
ggagacaggcagtcccggtagatc

SEQ ID NO: 34 - DNA, 24 bp, Artificial Sequence
  Goosecoid Reverse  Primer
tgggaattgtcccactctctgctc

SEQ ID NO: 35 - DNA, 5318 bp, Artificial Sequence
  Goosecoid Promoter
tagttattactagcgctaccggactcagatctcgagctcaagcttcgaattcgattggagacaggcagtcccggtagatcccacgagaattaaagccaaaaaaattttttttggggggggggaaggcaccccttctcccagactaataatcaaaaatacacagaacctatcggcacccacgcgggaagcctctggaagcatcgcttcagagcgctcctctgggtggtgaattttaaagactggctgaatttgctcttcactggtgtgttaggagctagggagagtcagggtagttttcagacccagagtgaccgctttaagaagaagaagaagaaaaaagacaacttgtacgaaggcggacgcgtttctactttcatggtttttgctctgagaaaatttggctttcgcaaaaacaaaagattttgggaaagagacggaggggggggtggagaagtgaaattaggcagtgagttcacggggtagggggttggctgcggggggggggcactttcagtcttagttgagggaggacacagccaccctcatttcttaaaagcaaacagattccgaaagagagtaaaaagtagtcctaaagtaaaattagccacaatgaatttgagctacaaccataggaaaccgcaccccataatagagagaaaagggtcggggcggagaggtcggcggcggagttgttaacggcggcaggacaatagtattaataagattaacctgggcaattaggccgcccgcccagcaaggccggggccgcgccggggctgccgaatggaaagattaggttaatttcattaattctcaatccacaatctttttcaggccctgtggcccccctcctcttggcatctctccccctcccctgcaagcgccccccgcccacccccacctccccattccacaccacccaaaggaaaagaaaaggaccaaatctggttctgtttgtcatctgcatattaccaggaactaaatccaggatgacgtcgactcagtataaaaccaacaagaggttgagccggtcggagctgcgtcctacccgcgggttgagttcagctaggcggcggcgaggggaggagagggcgggaggagggagttcggacgcagggggcggggaggggcgcgagttgcgcgctcgcccgcgctctctttcggtttgctcgcccgcgggagcagagagtgggacaattcccaaatcactagtgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 36 - DNA, 3930 bp, Artificial Sequence
  Goosecoid cDNA in pTRE
ctcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattagcttatgcccgccagcatgttcagcatcgacaacatcctggccgcccggccgcgctgcaaagacgcggtgctcccggtggcgcccagcgccgcggctccggtggtcttcccggctctacacggggactcgctctacggcgccggcggcggcacctcctcggactacggcgccttctacccgcgccctgtggcccccggaggcgcgggcctcccggccgcggtcggcagctcccgcctgggctacaacagctacttctacgggcagctgcacgtgcaggcggcgcccgtgggcccggcttgctgcggggctgtgccgccgctgggcgcccagcagtgctcctgcgtcccgacgcccccagcctaccagggccccggttctgtactggtgtctccggtgccgcaccagatgctgccctacatgaacgtgggcacgctgtcgcgcactgagctgcagctgctcaaccagctgcactgtcggcggaagcggcggcaccgcaccatcttcaccgatgagcagctcgaagccctggagaacctcttccaggagacgaagtacccagacgtgggcactcgggagcagctggccaggaaggtgcaccttcgggaggagaaggtggaggtctggtttaagaaccgccgagccaagtggagacgacagaagcgatcctcctcggaggagtcagaaaacgccgagaagtggaacaagacgtcctcaaaagcctcgccggagaagagggaagaggaaggtaaaagcgatttggactcggacagctgagaattcctgcagcccgggggatccactagttctagaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtctggccggaccacgctatctgtgcaaggtccccggacgcgcgctccatgagcagagcgcccgccgccgaggcaagactcgggcggcgccctgcccgtcccaccaggtcaacaggcggtaaccggcctcttcatcgggaatgcgcgcgaccttcagcatcgccggcatgtcccctggcggacgggaagtatcagctcgaccaagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 37 - DNA, 31 bp, Artificial Sequence
  Exon 1 Forward Primer (bp 296-316)
ggttaagcttatgcccgccagcatgttcagc

SEQ ID NO: 38 - DNA, 36 bp, Artificial Sequence
  Exon 1 Reverse Primer (bp 631-650)
gcggggccctcgtagcctgggggcgtcgggacgcag

SEQ ID NO: 39 - DNA, 21 bp, Artificial Sequence
  Exon 2 Forward Primer (bp 1165-1183)
cgagggccccggttctgtact

SEQ ID NO: 40 - DNA, 27 bp, Artificial Sequence
  Exon 2 Reverse Primer (bp 1398-1418)
tttgagctccaccttctcctcccgaag

SEQ ID NO: 41 - DNA, 21 bp, Artificial Sequence
  Exon 3 Forward Primer
gtctggtttaagaaccgccga

SEQ ID NO: 42 - DNA, 28 bp, Artificial Sequence
  Exon 3 Reverse Primer
ggaattctcagctgtccgagtccaaatc

SEQ ID NO: 43 - DNA, 3723 bp, Artificial Sequence
  pCMH142
ggtaccgggccccccctcgaggtcgacggtatcgataagcttatgcccgccagcatgttcagcatcgacaacatcctggccgcccggccgcgctgcaaagacgcggtgctcccggtggcgcccagcgccgcggctccggtggtcttcccggctctacacggggactcgctctacggcgccggcggcggcacctcctcggactacggcgccttctacccgcgccctgtggcccccggaggcgcgggcctcccggccgcggtcggcagctcccgcctgggctacaacagctacttctacgggcagctgcacgtgcaggcggcgcccgtgggcccggcttgctgcggggctgtgccgccgctgggcgcccagcagtgctcctgcgtcccgacgcccccagcctaccagggccccggttctgtactggtgtctccggtgccgcaccagatgctgccctacatgaacgtgggcacgctgtcgcgcactgagctgcagctgctcaaccagctgcactgtcggcggaagcggcggcaccgcaccatcttcaccgatgagcagctcgaagccctggagaacctcttccaggagacgaagtacccagacgtgggcactcgggagcagctggccaggaaggtgcaccttcgggaggagaaggtggaggtctggtttaagaaccgccgagccaagtggagacgacagaagcgatcctcctcggaggagtcagaaaacgccgagaagtggaacaagacgtcctcaaaagcctcgccggagaagagggaagaggaaggtaaaagcgatttggactcggacagctgagaattcctgcagcccgggggatccactagttctagagcggccgccaccgcggtggagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctg

SEQ ID NO: 44 - DNA, 30 bp, Artificial Sequence
  gsc F4 Forward Primer
ttaagcttgccaccatgcccgccagcatgt

SEQ ID NO: 45 - DNA, 28 bp, Artificial Sequence
  gsc R4 Reverse Primer
ttggatccgcgctgtccgagtccaaatc

SEQ ID NO: 46 - DNA, 5436 bp, Artificial Sequence
  pSFM6
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttgccaccatgcccgccagcatgttcagcatcgacaacatcctggccgcccggccgcgctgcaaagacgcggtgctcccggtggcgcccagcgccgcggctccggtggtcttcccggctctacacggggactcgctctacggcgccggcggcggcacctcctcggactacggcgccttctacccgcgccctgtggcccccggaggcgcgggcctcccggccgcggtcggcagctcccgcctgggctacaacagctacttctacgggcagctgcacgtgcaggcggcgcccgtgggcccggcttgctgcggggctgtgccgccgctgggcgcccagcagtgctcctgcgtcccgacgcccccagcctaccagggccccggttctgtactggtgtctccggtgccgcaccagatgctgccctacatgaacgtgggcacgctgtcgcgcactgagctgcagctgctcaaccagctgcactgtcggcggaagcggcggcaccgcaccatcttcaccgatgagcagctcgaagccctggagaacctcttccaggagacgaagtacccagacgtgggcactcgggagcagctggccaggaaggtgcaccttcgggaggagaaggtggaggtctggtttaagaaccgccgagccaagtggagacgacagaagcgatcctcctcggaggagtcagaaaacgccgagaagtggaacaagacgtcctcaaaagcctcgccggagaagagggaagaggaaggtaaaagcgatttggactcggacagcgcggatccaccggtcgccaccatggtgcgctcctccaagaacgtcatcaaggagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaagactcctccctgcaggacggctgcttcatctacaaggtgaagttcatcggcgtgaacttcccctccgacggccccgtaatgcagaagaagaccatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcaccgagggccgccaccacctgttcctgtagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 47 - DNA, 5604 bp, Artificial Sequence
  pSFM7
tagttattactagcgctaccggactcagatctcgagctcaagcttcgaattcgattggagacaggcagtcccggtagatcccacgagaattaaagccaaaaaaattttttttggggggggggaaggcaccccttctcccagactaataatcaaaaatacacagaacctatcggcacccacgcgggaagcctctggaagcatcgcttcagagcgctcctctgggtggtgaattttaaagactggctgaatttgctcttcactggtgtgttaggagctagggagagtcagggtagttttcagacccagagtgaccgctttaagaagaagaagaagaaaaaagacaacttgtacgaaggcggacgcgtttctactttcatggtttttgctctgagaaaatttggctttcgcaaaaacaaaagattttgggaaagagacggaggggggggtggagaagtgaaattaggcagtgagttcacggggtagggggttggctgcggggggggggcactttcagtcttagttgagggaggacacagccaccctcatttcttaaaagcaaacagattccgaaagagagtaaaaagtagtcctaaagtaaaattagccacaatgaatttgagctacaaccataggaaaccgcaccccataatagagagaaaagggtcggggcggagaggtcggcggcggagttgttaacggcggcaggacaatagtattaataagattaacctgggcaattaggccgcccgcccagcaaggccggggccgcgccggggctgccgaatggaaagattaggttaatttcattaattctcaatccacaatctttttcaggccctgtggcccccctcctcttggcatctctccccctcccctgcaagcgccccccgcccacccccacctccccattccacaccacccaaaggaaaagaaaaggaccaaatctggttctgtttgtcatctgcatattaccaggaactaaatccaggatgacgtcgactcagtataaaaccaacaagaggttgagccggtcggagctgcgtcctacccgcgggttgagttcagctaggcggcggcgaggggaggagagggcgggaggagggagttcggacgcagggggcggggaggggcgcgagttgcgcgctcgcccgcgctctctttcggtttgctcgcccgcgggagcagagagtgggacaattcccaaatcactagtgaattctgcagtcgacggtaccgcgggcccaccggtcgccaccatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtaggaattcgcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 48 - DNA, 6310 bp, Artificial Sequence
  pSFM20
tagttattactagcgctaccggactcagatctcgagctcaagcttcgaattcgattggagacaggcagtcccggtagatcccacgagaattaaagccaaaaaaattttttttggggggggggaaggcaccccttctcccagactaataatcaaaaatacacagaacctatcggcacccacgcgggaagcctctggaagcatcgcttcagagcgctcctctgggtggtgaattttaaagactggctgaatttgctcttcactggtgtgttaggagctagggagagtcagggtagttttcagacccagagtgaccgctttaagaagaagaagaagaaaaaagacaacttgtacgaaggcggacgcgtttctactttcatggtttttgctctgagaaaatttggctttcgcaaaaacaaaagattttgggaaagagacggaggggggggtggagaagtgaaattaggcagtgagttcacggggtagggggttggctgcggggggggggcactttcagtcttagttgagggaggacacagccaccctcatttcttaaaagcaaacagattccgaaagagagtaaaaagtagtcctaaagtaaaattagccacaatgaatttgagctacaaccataggaaaccgcaccccataatagagagaaaagggtcggggcggagaggtcggcggcggagttgttaacggcggcaggacaatagtattaataagattaacctgggcaattaggccgcccgcccagcaaggccggggccgcgccggggctgccgaatggaaagattaggttaatttcattaattctcaatccacaatctttttcaggccctgtggcccccctcctcttggcatctctccccctcccctgcaagcgccccccgcccacccccacctccccattccacaccacccaaaggaaaagaaaaggaccaaatctggttctgtttgtcatctgcatattaccaggaactaaatccaggatgacgtcgactcagtataaaaccaacaagaggttgagccggtcggagctgcgtcctacccgcgggttgagttcagctaggcggcggcgaggggaggagagggcgggaggagggagttcggacgcagggggcggggaggggcgcgagttgcgcgctcgcccgcgctctctttcggtttgctcgcccgcgggagcagagagtgggacaattcccaaatcactagtgaattctgcagtcgacggtaccgcgggcccgggatccaagctcagatctcgagctcggtacccgggtcgacaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 49 - DNA, 5143 bp, Artificial Sequence
  pSFM21
tagttattactagcgctaccggactcagatctcgagctcaagcttcgaattctgcagtcgacggtaccgcgggcccgggatccaagctcagatctcgagctcggtacccgggtcgacaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 50 - DNA, 5662 bp, Artificial Sequence
  pSFM23
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcggtacccgggtcgacaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 51 - DNA, 3871 bp, Artificial Sequence
  pSFM24
ctcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtctggccggaccacgctatctgtgcaaggtccccggacgcgcgctccatgagcagagcgcccgccgccgaggcaagactcgggcggcgccctgcccgtcccaccaggtcaacaggcggtaaccggcctcttcatcgggaatgcgcgcgaccttcagcatcgccggcatgtcccctggcggacgggaagtatcagctcgaccaagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 52 - DNA, 4824 bp, Artificial Sequence
  pSFM25
ctcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattctgcagtcgacaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtctggccggaccacgctatctgtgcaaggtccccggacgcgcgctccatgagcagagcgcccgccgccgaggcaagactcgggcggcgccctgcccgtcccaccaggtcaacaggcggtaaccggcctcttcatcgggaatgcgcgcgaccttcagcatcgccggcatgtcccctggcggacgggaagtatcagctcgaccaagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 53 - DNA, 18 bp, Artificial Sequence
  Forward Primer
gcggcaccgcaccatctt

SEQ ID NO: 54 - DNA, 20 bp, Artificial Sequence
  Reverse Primer
ggccgtcagctgtccgagtc

SEQ ID NO: 55 - DNA, 18 bp, Artificial Sequence
  Forward Primer
accggatttggccgtatt

SEQ ID NO: 56 - DNA, 21 bp, Artificial Sequence
  Reverse Primer
tctgggatggaaattgtggag

SEQ ID NO: 57 - DNA, 4386 bp, Artificial Sequence
  pSFM5
ctcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattagcttatgcccgccagcatgttcagcatcgacaacatcctggccgcccggccgcgctgcaaagacgcggtgctcccggtggcgcccagcgccgcggctccggtggtcttcccggctctacacggggactcgctctacggcgccggcggcggcacctcctcggactacggcgccttctacccgcgccctgtggcccccggaggcgcgggcctcccggccgcggtcggcagctcccgcctgggctacaacagctacttctacgggcagctgcacgtgcaggcggcgcccgtgggcccggcttgctgcggggctgtgccgccgctgggcgcccagcagtgctcctgcgtcccgacgcccccagcctaccagggccccggttctgtactggtgtctccggtgccgcaccagatgctgccctacatgaacgtgggcacgctgtcgcgcactgagctgcagctgctcaaccagctgcactgtcggcggaagcggcggcaccgcaccatcttcaccgatgagcagctcgaagccctggagaacctcttccaggagacgaagtacccagacgtgggcactcgggagcagctggccaggaaggtgcaccttcgggaggagaaggtggaggtctggtttaagaaccgccgagccaagtggagacgacagaagcgatcctcctcggaggagtcagaaaacgccgagaagtggaacaagacgtcctcaaaagcctcgccggagaagagggaagaggaaggtaaaagcgatttggactcggacagctgagaattcctgcagctcagtgcgcgacagcgtgcccacgttcatgtagggcagcatctggtgcggcaccggagacaccagtacagaaccggggccctggtaggctgggggcgtcgggacgcaggagcactgctgggcgcccagcggcggcacagccccgcagcaagccgggcccacgggcgccgcctgcacgtgcagctgcccgtagaagtagctgttgtagcccaggcgggagctgccgaccgcggccgggaggcccgcgcctccgggggccacagggcgcgggtagaaggcgccgtagtccgaggaggtgccgccgccggcgccgtagagcgagtccccgtgtagagccgggaagaccaccggagccgcggcgctgggcgccaccgggagcaccgcgtctttgcagcgcggccgggcggccaggatgttgtcgatgctgaacatgctggcgggcataagcttatcgataccgtcgacggtaccgcgggcccgggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtctggccggaccacgctatctgtgcaaggtccccggacgcgcgctccatgagcagagcgcccgccgccgaggcaagactcgggcggcgccctgcccgtcccaccaggtcaacaggcggtaaccggcctcttcatcgggaatgcgcgcgaccttcagcatcgccggcatgtcccctggcggacgggaagtatcagctcgaccaagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 58 - DNA, 4653 bp, Artificial Sequence
  pSFM8
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctcagctgtccgagtccaaatcgcttttaccttcctcttccctcttctccggcgaggcttttgaggacgtcttgttccacttctcggcgttttctgactcctccgaggaggatcgcttctgtcgtctccacttggctcggcggttcttaaaccagacctccaccttctcctcccgaaggtgcaccttcctggccagctgctcccgagtgcccacgtctgggtacttcgtctcctggaagaggttctccagggcttcgagctgctcatcggtgaagatggtgcggtgccgccgcttccgccgacagtgcagctggttgagcagctgcagctcagtgcgcgacagcgtgcccacgttcatgtagggcagcatctggtgcggcaccggagacaccagtacagaaccggggccctggtaggctgggggcgtcgggacgcaggagcactgctgggcgcccagcggcggcacagccccgcagcaagccgggcccacgggcgccgcctgcacgtgcagctgcccgtagaagtagctgttgtagcccaggcgggagctgccgaccgcggccgggaggcccgcgcctccgggggccacagggcgcgggtagaaggcgccgtagtccgaggaggtgccgccgccggcgccgtagagcgagtccccgtgtagagccgggaagaccaccggagccgcggcgctgggcgccaccgggagcaccgcgtctttgcagcgcggccgggcggccaggatgttgtcgatgctgaacatgctggcgggcataagcttatcgataccgtcgacggtaccgcgggcccaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 59 - DNA, 3926 bp, Artificial Sequence
  pSFM9
ctcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattctcagctgtccgagtccaaatcgcttttaccttcctcttccctcttctccggcgaggcttttgaggacgtcttgttccacttctcggcgttttctgactcctccgaggaggatcgcttctgtcgtctccacttggctcggcggttcttaaaccagacctccaccttctcctcccgaaggtgcaccttcctggccagctgctcccgagtgcccacgtctgggtacttcgtctcctggaagaggttctccagggcttcgagctgctcatcggtgaagatggtgcggtgccgccgcttccgccgacagtgcagctggttgagcagctgcagctcagtgcgcgacagcgtgcccacgttcatgtagggcagcatctggtgcggcaccggagacaccagtacagaaccggggccctggtaggctgggggcgtcgggacgcaggagcactgctgggcgcccagcggcggcacagccccgcagcaagccgggcccacgggcgccgcctgcacgtgcagctgcccgtagaagtagctgttgtagcccaggcgggagctgccgaccgcggccgggaggcccgcgcctccgggggccacagggcgcgggtagaaggcgccgtagtccgaggaggtgccgccgccggcgccgtagagcgagtccccgtgtagagccgggaagaccaccggagccgcggcgctgggcgccaccgggagcaccgcgtctttgcagcgcggccgggcggccaggatgttgtcgatgctgaacatgctggcgggcataagcttatcgataccgtcgacggtaccgcgggcccgggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctgcaagcctcgtcgtctggccggaccacgctatctgtgcaaggtccccggacgcgcgctccatgagcagagcgcccgccgccgaggcaagactcgggcggcgccctgcccgtcccaccaggtcaacaggcggtaaccggcctcttcatcgggaatgcgcgcgaccttcagcatcgccggcatgtcccctggcggacgggaagtatcagctcgaccaagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 60 - DNA, 1147 bp, Artificial Sequence
  Promoter
ggagacaggcagtcccggtagatcccacgagaattaaagccaaaaaaattttttttggggggggggaaggcaccccttctcccagactaataatcaaaaatacacagaacctatcggcacccacgcgggaagcctctggaagcatcgcttcagagcgctcctctgggtggtgaattttaaagactggctgaatttgctcttcactggtgtgttaggagctagggagagtcagggtagttttcagacccagagtgaccgctttaagaagaagaagaagaaaaaagacaacttgtacgaaggcggacgcgtttctactttcatggtttttgctctgagaaaatttggctttcgcaaaaacaaaagattttgggaaagagacggaggggggggtggagaagtgaaattaggcagtgagttcacggggtagggggttggctgcggggggggggcactttcagtcttagttgagggaggacacagccaccctcatttcttaaaagcaaacagattccgaaagagagtaaaaagtagtcctaaagtaaaattagccacaatgaatttgagctacaaccataggaaaccgcaccccataatagagagaaaagggtcggggcggagaggtcggcggcggagttgttaacggcggcaggacaatagtattaataagattaacctgggcaattaggccgcccgcccagcaaggccggggccgcgccggggctgccgaatggaaagattaggttaatttcattaattctcaatccacaatctttttcaggccctgtggcccccctcctcttggcatctctccccctcccctgcaagcgccccccgcccacccccacctccccattccacaccacccaaaggaaaagaaaaggaccaaatctggttctgtttgtcatctgcatattaccaggaactaaatccaggatgacgtcgactcagtataaaaccaacaagaggttgagccggtcggagctgcgtcctacccgcgggttgagttcagctaggcggcggcgaggggaggagagggcgggaggagggagttcggacgcagggggcggggaggggcgcgagttgcgcgctcgcccgcgctctctttcggtttgctcgcccgcgggagcagagagtgggacaattccca

SEQ ID NO: 61 - DNA, 771 bp, Artificial Sequence
  Blocker Molecule
atgcccgccagcatgttcagcatcgacaacatcctggccgcccggccgcgctgcaaagacgcggtgctcccggtggcgcccagcgccgcggctccggtggtcttcccggctctacacggggactcgctctacggcgccggcggcggcacctcctcggactacggcgccttctacccgcgccctgtggcccccggaggcgcgggcctcccggccgcggtcggcagctcccgcctgggctacaacagctacttctacgggcagctgcacgtgcaggcggcgcccgtgggcccggcttgctgcggggctgtgccgccgctgggcgcccagcagtgctcctgcgtcccgacgcccccagcctaccagggccccggttctgtactggtgtctccggtgccgcaccagatgctgccctacatgaacgtgggcacgctgtcgcgcactgagctgcagctgctcaaccagctgcactgtcggcggaagcggcggcaccgcaccatcttcaccgatgagcagctcgaagccctggagaacctcttccaggagacgaagtacccagacgtgggcactcgggagcagctggccaggaaggtgcaccttcgggaggagaaggtggaggtctggtttaagaaccgccgagccaagtggagacgacagaagcgatcctcctcggaggagtcagaaaacgccgagaagtggaacaagacgtcctcaaaagcctcgccggagaagagggaagaggaaggtaaaagcgatttggactcggacagctga

SEQ ID NO: 62 - DNA, 779 bp, Artificial Sequence
  Blocker Molecule
catccgtcggttttggaaccagatcttcacttgcctttcggagagagcgagattttgggccagctcggctttcctcctgatggtgatgtagcgactgtagaggaactccttttccagctccagcgtctgaatcgaattcatgaggaacttaggagacgacgggaacgcagaccggccacagcgcttcctcctccggaactgactgatcatggtcgccgtggtccgcgctctcacggtgctgttgctcggtcaggtgttgctgggaggtgccgttggactcattcccgagatcgaccgacggaaatacagtgattcggggagacacacaccggagcgaactgatacaaacttcctgaacgagtttgagctacgcttgctcaatatgttcggattgaagcgaaaacccaccccaagcaaatcggcagtggtccctcagtacatgctggacttgtattatatgcactctgaaaacgatgacccgaacattcggcgcccgaggagcactatgggaaaacatgtagaaagggcagccagcagagcaaacacgatacgaagttttcatcacgaagaggctttcgaggcactgtccagcctgaaaggaaaaacaacgcagcagtttttcttcaaccttacctccattcctgtcgactcagacgctggagctggaaaaggagttcctctacagtcgctacatcaccatcaggaggaaagccgagctggcccaaaatctcgctctctccgaaaggcaagtgaagatctggttccaaaaccgacggatg

SEQ ID NO: 63 - DNA, 1432 bp, Artificial Sequence
  Gene
gaattcatgaggaacttaggagacgacgggaacgcagaccggccacagcgcttcctcctccggaactgactgatcatggtcgccgtggtccgcgctctcacggtgctgttgctcggtcaggtgttgctgggaggtgccgttggactcattcccgagatcgaccgacggaaatacagtgattcggggagacacacaccggagcgaactgatacaaacttcctgaacgagtttgagctacgcttgctcaatatgttcggattgaagcgaaaacccaccccaagcaaatcggcagtggtccctcagtacatgctggacttgtattatatgcactctgaaaacgatgacccgaacattcggcgcccgaggagcactatgggaaaacatgtagaaagggcagccagcagagcaaacacgatacgaagttttcatcacgaagaggctttcgaggcactgtccagcctgaaaggaaaaacaacgcagcagtttttcttcaaccttacctccattcctggcgaggagctgatctccgctgcggagctgcgcattttcagggaccaagttctcggagatgccagtacgagtggcttccacagaattaacatttacgaggtgttcaggccagctttggccccctccaaagagcctctaaccagacttctggacacccgtctggtgcaggactctcacacgcgctgggaaagcttcgacgtgggttcagctgtggcacgctgggcccgcgaatcccagcacaaccacgggctccttgtagaggtgctccatcctaaggagtcagaagtatccgaggaggctgagagcaaccggaggaagcacgtgagggtcagtcgttcccttcacgcggatgaggactcgtgggcacaagcccgacctctgctggtaacctacagccatgacggtcaaggcacagccgtcttgcattcgaaccgagaaaagcggcaggctcgacgagggcaaaagccgaggagaaagcaccaccagcgctcgaactgtaggcgacatgctctctatgtggacttcagtgatgtcggctggaacgagtggatcgtggcaccgccaggctatcatgctttctactgccatggcgagtgtccgttccctctgccggaccatctaaactccaccaaccatgccattgtccagacgctggtgaactcggtcaactccaacattcccaaagcctgttgcatcccgacggagctcagccctatctcactgctgtacctggacgagtacgagaaggtcattcttaaaaactaccaggacatggtggtggagggctgtgggtgccgatgagaacaatctccccaatgaagacttttatttatacaaaagagcgagctatttggaggaagaaaagaaatatatatgaatatatttatgttgaatgaacaaaacaaaaaaaaaaaaaaaaaaa